SEQUENCE LISTING (36 sequences)

SEQ ID NO: 1 - RNA, 151 nt, Homo sapiens
  source 1..151: mol_type = other RNA
gtgaccatcacaggcaggctgtgcccacagcgtgggatgtgcaagtctgtgtttgtgatggaggccggggaggccgctgaccccaccacggtcctgtgctctgtggaggagctggcactggcccattacagacgcagcggttttgaccagg

SEQ ID NO: 2 - RNA, 2613 nt, Homo sapiens
  source 1..2613: mol_type = other RNA
gtaactgagcaggctttctcttgtggcacccagccccgggtggacgagcagcagcactggatgggctgtcagcagtgggctgctgtcctctctctgtcctctgctcacagtggaagatgctgtgggctgggggatgtcttcctattcttccccttatcaatgataaggagtaggtcaggggtagttgtcccacagggcctggctctgatcttttttcttttataaaaacttataaaagttatggaaggtactgcacaagcctttgtacagaagtcagggggacagtgtgctcaaccccacggacacccacttccattcagtgttccttctgttctctcctgccccttccctgggattttagaagctggccccagatatcctctcaggggccctgcggcccatgctagtcgctttcgggaacagccactgagccttccgcccttgggcctggcaggactctccctgtgcccgggctgcagggttaccagtgttaaaaacactgatattttcagatgtgtgaaaatcaggacatgatttcagtattccccgagtgttcggaagattaacaagtactatagaaagacaaccacaagataggagtttacaaagcccaggttctttctgatgagtgttgagattcatctacataatatctttattacttacagggaagggtgatgttattggggaagacgtgcaaatgccagtgaagacagagagatacagtaggcctgaaatggttagaaatagaagaatagaatgcacattcaaggccccgagagacgtggaaactggacactcgctggaggaggaagcaggctgggacgtgggagcgctgaaatgctttcagttgagccatgagcctgaaaaacacacgattcctttcccagagtagaaactatttttccctgattaaaatcgatgccgtgaaactgggctgaactctggctgccaggctttatgccacagggaattttgtgtcagagcgatctcaacctcttctgggaggtgatttctgaagtactgcaccaaaaatgatttcacttataacacaaatgcacagcatggaccacttaggagttaaggaacgaaccaggcacggtagcttacacttgtaatcccagcacttggggaggctgagacaggaggatcacttgaactctgaagacggaggtgggaggatctcttgaccccaggaggtcaaggctgcagtgagccaagattgtgccactgcactccagtctgggtgacagagtgagaccctgtctcaaaacaaaacaaacaaaggagttcagaaatgatctggcctttatattcctgcctgatcgtcagtgtattcaccaggacggaacactgactggaaatcaggtcctagctccactcctggctgggaaagagcatgaagtgttctaggaagaaaagtcctcctggaagacttggcaacagccaggagtcctgagtgaaccgctgtccttttgaatccctgagacgggctgcagggatgaggggctagaactgacaggaagacaggacagagagcagcacagcctcagaagtgcaggacagaggtgacccaggcaggcatgacgggctgtggcttcctgccctctgctctcacagcacccctgatcccactcactgtggtaccacgcagatgggttggggcctgtaaatgtcaggtgggaccagccagaggagctttgctgccagtactggcatgtcaggatgggggtctggtggtcagcacctcctctggccagtggtcttgtggggagaggcatccatgtggcctcctcctctgtcgggagggctgatgtgcagagaagtacggacttggagggcagctggatgagctggggcttgctcagggacacaggtctgtgtgttgtgccaaggctgaccccctcatcccacaccagaccctgcctctgactctgtgacctcagcacctgacttctgtctctcaggtcatctcccagtcagtaaaacatttgtgtgacgtgaggaggggcactgaagtgggtgatctttaaggccagatgtctctgtaaaagccttgactatcggaagcactgggctagggcagtgtagtacccagggggatgaggtgcatcagagcagcacctgctgaggccaagaacggaggtggctgctgccggcctccgggaggtccccttagggaagcatttcttcaggggtggcccagggacctccggcatcagggcctgcatggggattctgtgccagcccagccctgctaagtcccagccttgggaacctgaggagggtatctgcctatttttcttttaaacatgtttcttaggttattcactaaagtttgagaaccactgctttgtggctttgaaatgttcatttgaatttttctatgattactaagatttttgcccttaaggctctgtatataaacaacatactgtataaaataaactgggaatggcgtgaaaacagcatttgcttctgaatctaaaatatttctattattttcttgaaattgagtgtgcagtaggttatggtggtgttttggaagaaacagataaaacagattttgtgtgtgtgtgtgtgtgtgtgtgtgtgtgtgtgaccttgtcttag

SEQ ID NO: 3 - RNA, 136 nt, Homo sapiens
  source 1..136: mol_type = other RNA
ggattcatggcgaagggtccaccttcagcaccctgtatggcctcctcctgtgggacatcatcttcatggatgggattccggatgtcttcagaaacgcctgtcaggtactccagtgcccctgccccacgagtaggtc

SEQ ID NO: 4 - RNA, 19 nt, Homo sapiens
  source 1..19: mol_type = other RNA
ttacttacagggaagggtg

SEQ ID NO: 5 - RNA, 19 nt, Homo sapiens
  source 1..19: mol_type = other RNA
ggtgatgttattggggaag

SEQ ID NO: 6 - RNA, 21 nt, Homo sapiens
  source 1..21: mol_type = other RNA
tttattacttacagggaaggg

SEQ ID NO: 7 - DNA, 19 bp, synthetic construct
  source 1..19: mol_type = other DNA
  modified_base 1: mod_base = m5c
  modified_base 3..5: mod_base = m5c
  modified_base 8..10: mod_base = m5c
  modified_base 6..7: mod_base = m5u
  modified_base 11: mod_base = m5u
  modified_base 13: mod_base = m5u
  modified_base 17: mod_base = m5u
cacccttccctgtaagtaa

SEQ ID NO: 8 - DNA, 19 bp, synthetic construct
  source 1..19: mol_type = other DNA
  modified_base 2..3: mod_base = m5u
  modified_base 10: mod_base = m5u
  modified_base 15: mod_base = m5u
  modified_base 1: mod_base = m5c
  modified_base 4..7: mod_base = m5c
  modified_base 13: mod_base = m5c
  modified_base 16: mod_base = m5c
  modified_base 18..19: mod_base = m5c
cttccccaataacatcacc

SEQ ID NO: 9 - DNA, 21 bp, synthetic construct
  source 1..21: mol_type = other DNA
  modified_base 1..3: mod_base = m5c
  modified_base 6..8: mod_base = m5c
  modified_base 4..5: mod_base = m5u
  modified_base 9: mod_base = m5u
  modified_base 11: mod_base = m5u
  modified_base 15: mod_base = m5u
  modified_base 18: mod_base = m5u
cccttccctgtaagtaataaa

SEQ ID NO: 10 - DNA, 17 bp, synthetic construct
  source 1..17: mol_type = other DNA
  modified_base 2: mod_base = m5c
  modified_base 8: mod_base = m5c
  modified_base 10: mod_base = m5c
  modified_base 12: mod_base = m5c
  modified_base 14: mod_base = m5c
  modified_base 16: mod_base = m5c
  modified_base 7: mod_base = m5u
acaaggtcacacacaca

SEQ ID NO: 11 - DNA, 18 bp, synthetic construct
  source 1..18: mol_type = other DNA
  modified_base 4: mod_base = m5u
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
aggtcacacacacacaca

SEQ ID NO: 12 - DNA, 18 bp, synthetic construct
  source 1..18: mol_type = other DNA
  modified_base 2: mod_base = m5u
  modified_base 3: mod_base = m5c
  modified_base 5: mod_base = m5c
  modified_base 7: mod_base = m5c
  modified_base 9: mod_base = m5c
  modified_base 11: mod_base = m5c
  modified_base 13: mod_base = m5c
  modified_base 15: mod_base = m5c
  modified_base 17: mod_base = m5c
gtcacacacacacacaca

SEQ ID NO: 13 - DNA, 25 bp, synthetic construct
  source 1..25: mol_type = other DNA
  modified_base 1..2: mod_base = m5c
  modified_base 4: mod_base = m5c
  modified_base 8..9: mod_base = m5c
  modified_base 11: mod_base = m5c
  modified_base 17: mod_base = m5c
  modified_base 3: mod_base = m5u
  modified_base 5..6: mod_base = m5u
  modified_base 10: mod_base = m5u
  modified_base 14..15: mod_base = m5u
  modified_base 20..22: mod_base = m5u
  modified_base 24: mod_base = m5u
cctcttacctcagttacaatttata

SEQ ID NO: 14 - DNA, 7079 bp, synthetic construct
  source 1..7079: mol_type = other DNA
tgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttacctggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgcgccaccatggtgtttgtgatggaggccggggaggccgctgaccccaccacggtcctgtgctctgtggaggagctggcactggcccattacagacgcagcggttttgaccagggtaactgagcaggctttctctcgtggcacccagccccgggtggacgagcagcagcactggatgggctgtcagcagtgggctgctgtcctctctctgtcctctgctcacagtggaagatgcctatagaaagacaaccacaagataggagtttacaaagcccaggttctttctgatgagtgttgagattcatctacataatatctttattacttacagggaagggtgatgttattggggaagacgtgcaaatgccagtgaagacagagagatacagtaggcctggcccttaaggctctgtatataaacaacatactgtataaaataaactgggaatggcgtgaaaacagcatttgcttctgaatctaaaatatttctattattttcttgaaattgagtgtgcagtaggttatggtggtgttttggaagaaacagataaaacagattttgtgtgtgtgtgtgtgtgtgtgtgtgtgtgtgtgaccttgtcttagggattcatggcgaagggtccaccttcagcaccctgtatggcctcctcctgtgggacatcatcttcatggatgggattccggatgtcttcagaaacgcctgtggtggtggttccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaagcttagccatggcttcccgccggcggtggcggcgcaggatgatggcacgctgcccatgtcttgtgcccaggagagcgggatggaccgtcaccctgcagcctgtgcttctgctaggatcaatgtgtaggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgccacaacccgggatccaccggtcgccaccatggtgagcaagggcgaggaggtcatcaaggagttcatgcgcttcaaggagcacatggagggctccgtgaacggccacgagttcgagatcgagggcgagggcgagggccgcccctacgagggcacccagaccgccaggctgaaggtgaccaagggtggccccctgcccttcgcctgggacatcctgtcccctcagatcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaaggtgcgcggcaccaacttcccctccgacggccccgtaatgcagaagaagaccatgggctgggaggcctcctccgagcggatgtaccccgaggacggcgccctgaagggcgagatgaagatgaggctgaggctgaaggacggcggccactacgacgccgaggtcaagaccacctacatggccaagaagcccgtgcagctgcccggcgcctacaagaccgacatcaagctggacatcacctcccacaacgaggactacaccatcgtggaacagtacgagcgcgccgagggccgccactccaccggcgccagatctcgagctcaagcttcgaattctgcagtcgacggtaccgcgggcccgggatccaccggatctagataactgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggcagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagt

SEQ ID NO: 15 - DNA, 504 bp, synthetic construct
  source 1..504: mol_type = other DNA
atggcaaacctggacaagatgctgaataccacagtaacagaggtgcggcagttcctgcaggtggacagagtgtgcgtgttccagtttgaggaggattatagcggagtggtggtggtggaggccgtggacgataggtggatctccatcctgaagacccaggtgcgggatagatacttcatggagacaaggggcgaggagtattctcacggccgctaccaggccatcgccgacatctacaccgcaaacctgacagagtgctacagggatctgctgacacagtttcaggtgagagcaatcctggccgtgcccatcctgcagggcaagaagctgtggggcctgttggtggcacaccagctggcggcccctagacagtggcagacctgggagatcgactttctgaagcagcaggccgtggtggtgggcatcgccatccagcagagcggaggtggtggatcaggaggaggtggttctggaggtggtggatcaggaggaagatctcgagct

SEQ ID NO: 16 - DNA, 212 bp, synthetic construct
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 17 - DNA, 514 bp, synthetic construct
  source 1..514: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 18 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 19 - DNA, 753 bp, synthetic construct
  source 1..753: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 20 - DNA, 404 bp, synthetic construct
  source 1..404: mol_type = other DNA
atccccggtccgcgctccgcccacccatcacagcagccgcggacgctgcgcgccggagcggtccatctcgccagccagccaaccagccgagccgcccagccgacccgagagccccgagagccagactccctcagccatagaagacaccgggcgggagagacggactgaaaaaatatatctttttttattttgtctgggcctggagacccgcagcaggagcggaggtgggtgcggggccgggagccggagcaggaccgggaacaggaacaggaacaggaacaggaacaggaacaggagtggggccgggagcaggagcaggagcgggagccgaagtgggggcaggagcggcggcggccgcagcagcaacagggtcgccccagtccgcggcgaggaagagggagctc

SEQ ID NO: 21 - DNA, 4371 bp, synthetic construct
  source 1..4371: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggatctgccgccaccatggcaaacctggacaagatgctgaataccacagtaacagaggtgcggcagttcctgcaggtggacagagtgtgcgtgttccagtttgaggaggattatagcggagtggtggtggtggaggccgtggacgataggtggatctccatcctgaagacccaggtgcgggatagatacttcatggagacaaggggcgaggagtattctcacggccgctaccaggccatcgccgacatctacaccgcaaacctgacagagtgctacagggatctgctgacacagtttcaggtgagagcaatcctggccgtgcccatcctgcagggcaagaagctgtggggcctgttggtggcacaccagctggcggcccctagacagtggcagacctgggagatcgactttctgaagcagcaggccgtggtggtgggcatcgccatccagcagagcggaggtggtggatcaggaggaggtggttctggaggtggtggatcaggaggaagatctcgagctatgtcagaagggaaacctcctgacaaaaaaaggcctcgtagaagcttatcaatcagcaagaataagaaaaaagcatctaattctattatttcgtgttttaacaatgcaccacctgctaaacttgcctgccccgtttgcagtaaaatggtgcctagatatgacttaaaccggcaccttgatgaaatgtgtgctaacaatgacttcgttcaagtggatccagggcaggttggcttaataaattcaaatgtgtctatggtagatttaaccagtgttaccttagaagatgtaacacctaagaagtcaccaccaccaaagacaaatttaacccctggccaaagtgattcagcaaaaagggaagtaaagcagaagatcagtccctactttaaaagtaatgatgtggtgtgcaaaaatcaagatgagctgagaaatcgtagtgtgaaagtcatttgtttgggaagcctagcatctaaattgtccagaaaatacgtaaaggctaaaaaatcaatagataaggatgaagaatttgccggttctagtccacagagttccaaatgcacagttgttaagagcctgattgataactcttcagaaattgaggacgaggatcaaattttggagaacagttctcaaaaagaaaacgtgtttaaatgtgattctctaaaggaagagtgcattcctgaacatatggtaagaggaagtaaaataatggaagccgaaagccaaaaggctacccgggaatgtgagaaatcagccctcacccctggattctcagataatgcgatcatgttattctcaccagatttcactcttaggaatacattaaagtctacttcagaagacagtcttgtaaagcaagagtgtatcaaagaagtggttgaaaaacgtgaggcatgtcattgtgaagaagtaaaaatgactgttgcttcagaagctaaaatacagctgtcagattcagaggcaaaatctcatagttctgcagatgatgcttctgcatggagtaacatccaagaggctcctctgcaggatgacagttgcttaaacaatgatatccctcacagcattcctttggagcaggggtcaagctgcaatggtcctggtcaaacaaccggtcatccttactaccttcggagtttccttgtggtgctgaaaaccgtacttgagaatgaagatgatatgttgctctttgatgagcaggagaagggaattgtaactaaattttatcagttatcagctactggtcagaagttatatgtaaggctctttcaacgtaaattaagctggattaagatgaccaaattagagtatgaagagattgccttagacttaacacctgtgattgaagaattgacgaatgcaggctttctacagacagaatctgagttgcaagaactctctgaagtgcttgaactcctttctgctcctgaactaaaatccctagccaagaccttccacttggtgaatcccaatggacagaaacagcagctggtggacgcctttctcaaattggccaaacagcgttcagtctgcacttggggcaagaataagcctggaattggtgcagtgattttaaaaagagccaaagccttggctggacagtcagtacgaatctgtaaaggccccagggctgtgttttcccgcatcttgctactgttttcgttgaccgactcaatggaagatgaagacgccgcttgtggaggtcagggacagctttcaacagtcctgttggtcaacctcggccgaatggagtttcctagttacaccatcaatcggaaaacccacatcttccaagacagagatgatcttatcagatatgcagcagccacgcacatgctgagtgacatttcttccgcaatggccaatgggaactgggaagaagctaaggagctcgctcagtgtgcaaaaagggattggaacagactgaaaaaccacccttctctgagatgccacgaagatttaccactcttcctgcggtgtttcactgttgggtggatttatacaaggattttgtctcggtttgtggaaatactgcagagacttcacatgtatgaggaagccgtcagagaacttgaaagccttttgtctcagagaatttattgtcctgacagcagaggccgatggtgggatcgactggcccttaatttacaccagcacttgaagcgcctggaaccgactatcaagtgcatcacagaggggctggcggatccggaagtcagaacgggacaccgcctttcactgtatcagcgagccgtgcgcctgcgagagtctccgagctgtaaaaagttcaagcacctcttccagcagctcccagaaatggctgtgcaagatgtgaaacacgtgaccatcacaggcaggctgtgcccacagcgtgggatgtgcaagtctgtgtttgtgatggaggccggggaggccgctgaccccaccacggtcctgtgctctgtggaggagctggcactggcccattacagacgcagcggttttgaccaggggattcatggcgaagggtccaccttcagcaccctgtatggcctcctcctgtgggacatcatcttcatggatgggattccggatgtcttcagaaacgcctgtcaggcattccccctggacttgtgcacagacagcttcttcacaagcagacgcccagcccttgaggccaggctgcagctgattcatgatgcccccgaggagagcctgcgggcctgggtggcagccacgtggcatgagcaggaaggcagagtggcttcccttgtcagctgggatcgcttcacgtctcttcagcaagctcaggatcttgtctcctgcctggggggccctgtgctcagtggtgtgtgcaggcacctggctgctgactttcgacactgtcgagggggcctccccgacctggtggtgtggaactcccagagccgtcactttaagctggtggaagttaaaggccccaatgatcgtctttcacataagcagatgatctggctggctgaactgcagaagctgggggctgaagtagaagtctgccatgtggttgcagttggagctaagagccaaagccttagctaagaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 30 bp, synthetic construct
  source 1..30: mol_type = other DNA
atgaaggccttcgagtccctcaagtccttc

SEQ ID NO: 23 - DNA, 21 bp, synthetic construct
  source 1..21: mol_type = other DNA
ggcggctgaggaagctgagga

SEQ ID NO: 24 - RNA, 150 nt, Homo sapiens
  source 1..150: mol_type = other RNA
ttctgaatctaaaatatttctattattttcttgaaattgagtgtgcagtaggttatggtggtgttttggaagaaacagataaaacagattttgtgtgtgtgtgtgtgtgtgtgtgtgtgtgtgtgaccttgtcttagggattcatggcga

SEQ ID NO: 25 - RNA, 160 nt, synthetic construct
  source 1..160: mol_type = other RNA
ttctgaatctaaaatatttctattattttcttgaaattgagtgtgcagtaggttatggtggtgttttggaagaaacagataaaacagattttgtgtgtgtgtgtgtgtgtgtgtgtgtgtgtgtgtgtgtgtgtgaccttgtcttagggattcatggcga

SEQ ID NO: 26 - DNA, 20 bp, synthetic construct
  source 1..20: mol_type = other DNA
  modified_base 1..2: mod_base = OTHER; note = 2'MOE Adenosine
  modified_base 3..4: mod_base = OTHER; note = 2'MOE Guanosine
  modified_base 5: mod_base = OTHER; note = 2'MOE ME Cytosine
  modified_base 16: mod_base = OTHER; note = 2'MOE Adenosine
  modified_base 17: mod_base = OTHER; note = 2'MOE ME Cytosine
  modified_base 18..20: mod_base = OTHER; note = 2'MOE Thymidine
aaggcttcatattgtacttt

SEQ ID NO: 27 - DNA, 3054 bp, synthetic construct
  source 1..3054: mol_type = other DNA
atgatgagcgagggcaagcctccagacaaaaagagacctagaagaagcctgtccatcagcaagaacaagaagaaggccagcaacagcatcatctcttgctttaacaacgccccacctgccaagctggcctgtcctgtttgtagcaaaatggtgcccagatacgacctgaataggcacctggacgaaatgtgcgccaacaacgacttcgtgcaggtcgaccctggccaagtgggcctgatcaactccaacgtgtccatggtggatctgacctctgtgaccctggaagatgtgacccctaagaagtcccctccaccaaagacaaacctgacccctggccagagcgactccgccaagcgagaagtgaagcagaagatctccccctacttcaagagcaacgacgtggtgtgcaagaaccaggacgagctgcggaatcggagcgtgaaagtgatctgcctgggctccctggcctccaagctgtcccgaaagtacgtgaaggccaagaaatctatcgacaaggacgaggaattcgccggcagttcccctcagtcctccaagtccaccgtggtgaagagcctgatcgacaattcctccgagatcgaggacgaggatcagatcctggagaacagctcccagaaggagaacgtgtttaagtgtgacagcctgaaggaggagtgcatccctgagcacatggtgagaggcagcaagatcatggaagccgagtctcagaaggccaccagagaatgcgagaagagtgccctgacaccaggcttctccgacaatgccatcatgcttttctcccccgatttcacactgagaaacaccctgaagtccaccagcgaggacagcctggtgaagcaggagtgtatcaaagaggtggtggagaagagagaggcctgtcactgtgaggaggtgaagatgacagtggcctccgaggccaagattcagctgtcagactctgaagccaagtctcatagcagcgccgatgatgcctctgcctggtccaacattcaggaggcccctctgcaggatgacagctgcctgaacaacgatatcccccacagcatccctctggaacagggcagcagctgtaatggtcctggacagaccaccgggcatccttactacctgcgctctttcctggtggtgctgaaaaccgtgctggagaacgaggacgatatgctgctgttcgacgagcaggagaagggcatcgtgaccaagttctaccagctgtccgccaccggccagaagctgtacgtgcggctgtttcagagaaagctgagctggatcaagatgaccaaactggagtacgaagagatcgccctggacctgacacccgtgatcgaggagctgaccaacgccggctttctgcagaccgagtccgagctgcaggagctgtccgaagtgctggagctgctgagcgctcctgagctgaagagcctggccaagaccttccacctggtgaatcccaacgggcagaagcagcagctggtggacgcctttctgaagctggccaagcagagaagcgtgtgcacctggggcaagaacaagcctggcatcggggccgtgatcttgaaaagagccaaagccctggccggacagtccgtgcgcatctgcaagggccctagggccgtgttcagcaggatcttgctgttgttctccttgaccgactccatggaggacgaggacgccgcatgtggcggtcagggacagctgtccacagtgctgctggtcaacctgggcagaatggagttccctagttacaccatcaacagaaaaacccacatcttccaggaccgagatgacctgatcagatacgccgccgccacccatatgctgagcgacatctcttccgccatggccaatggcaactgggaagaggccaaggaactggcccagtgcgccaagagagattggaacaggctgaagaaccacccttccctgagatgccacgaagatctgcccctgttcctgcgatgcttcaccgttggctggatctacacaagaatcctgagccggttcgtggaaatcctgcagaggctgcacatgtacgaggaagccgtcagagaactggagagcctgctgtcccagagaatctactgccccgactccagaggcagatggtgggaccgactggcccttaatctgcaccagcacctgaagcgactggagcctactatcaagtgcatcaccgagggcctggccgacccagaggtgcgaaccggacacagactgagcctgtaccagcgggccgtgcgactgagagagagccccagctgcaagaagttcaagcacctgttccagcagctgcccgagatggcagtgcaagatgtgaaacacgtgaccatcacaggcagactgtgccctcagaggggcatgtgcaagtctgtgtttgtgatggaagctggcgaggccgccgaccccaccacagttctgtgcagcgtggaggagctggctctggcccactacagaagatccggtttcgaccagggcattcacggcgagggcagcacatttagcaccctgtatggcctgctgctgtgggacatcatcttcatggatgggatccccgacgtgttcagaaacgcctgtcaggccttccccctggacctgtgcaccgacagcttcttcacctccagaagacccgctctggaggccagactgcaactgatccacgacgcccctgaggagtccctgagagcctgggtggccgccacctggcacgaacaggagggcagagtggctagcctggtctcctgggatagattcacctccctgcagcaggcccaggatctggtgagctgcctgggaggccccgtgctgagcggcgtgtgcagacatctggccgccgatttccgacactgcagaggcggccttcctgatctggtggtgtggaacagccagagccggcactttaaactggtcgaagtgaagggccccaacgatcgactgagccacaaacagatgatttggctggccgagctgcagaaactgggcgccgaggtggaagtgtgccatgtggtggccgtgggcgccaagtctcagtccctgagctaa

SEQ ID NO: 28 - DNA, 3054 bp, synthetic construct
  source 1..3054: mol_type = other DNA
atgatgagtgagggtaagccgccagataagaagcgaccgagacgttccctaagtatttctaaaaataagaaaaaagcctcgaattcgatcatctcctgcttcaataatgcaccaccagcaaagctcgcctgtcctgtttgctccaaaatggtgccaagatacgacttaaatcgtcatctagacgaaatgtgcgcaaacaatgatttcgtccaagtcgatccgggccaagtaggtctaattaattccaatgtctcgatggttgacctcacttctgtcacattagaagacgttacccccaaaaagtctcctccgccgaagactaatctaacgccaggccagagtgatagtgccaaacgagaggtaaagcagaaaatctctccgtattttaaatcgaatgacgttgtttgtaaaaaccaggacgaactacgtaatcgttcggtcaaggttatttgcttgggatccctcgcttccaagctttcgcgtaaatatgtcaaagcaaagaaatctattgacaaggatgaggagtttgccggaagttcccctcagtcctcgaagtcgacggtcgtaaagagtctcatcgataattcgtccgaaatcgaagacgaagatcaaatactcgaaaactcttcccaaaaagaaaacgtcttcaaatgcgacagtctaaaagaagagtgcatccccgaacacatggttagagggtcgaagatcatggaagcagagtctcagaaagcaacgagagaatgcgaaaagagtgctttgacaccgggattctcggacaatgcaatcatgttattttctccagatttcacattacgtaatacattgaagtctacatcggaggattccctcgttaagcaggaatgcattaaagaagtagtagagaaaagagaagcatgtcactgcgaagaggttaaaatgaccgtggcttctgaagctaagattcagctctccgactcggaagccaaatctcatagtagtgctgatgatgcctctgcctggtcgaatattcaggaagccccactccaggatgattcgtgtctgaacaatgatataccgcattccatcccgcttgaacagggctcctcctgcaacggcccaggacagactactggtcatccttactatttgcgcagtttccttgtcgtcctaaaaactgtcctcgaaaacgaagacgatatgctcttatttgacgaacaagagaaaggtattgtcacgaaattttaccaactctcggctactggccaaaaattgtatgtgagattatttcaaagaaaattgagttggataaaaatgacaaaattggaatacgaagaaatcgccctcgacctcacaccagtcattgaagaactcacaaatgccggatttctccagactgaaagtgaattgcaagagctctctgaggtactcgagctactctccgctcccgagttgaaatccctagccaaaacattccatttagtaaatcccaatggacaaaaacaacagttggtagacgcctttttaaagcttgccaagcaaagaagtgtctgcacgtggggaaaaaacaagcccggcatcggtgccgtcattttaaaacgtgccaaggctttagcaggccaatcggtccgtatctgcaagggtccaagagcagtgttctcccgtatcttgctactcttttcgcttacggattccatggaggacgaagatgctgcatgcggaggtcagggacaactttcgacggttttattggtcaacctcggaagaatggagttcccctcttacaccataaatagaaaaacccacatttttcaagatcgagatgatctcatccgttatgctgctgccacgcatatgctaagcgatattagttctgctatggccaacggaaattgggaagaagccaaggaactagcacaatgcgccaagcgtgattggaaccgcctaaagaaccacccttcgttgagatgccacgaagacctcccactcttcttaagatgcttcactgttggctggatatatacaagaatcctatctcgtttcgttgaaatcctacaaagactacacatgtatgaagaagccgtccgtgaattggagtcgttgctttctcagagaatttattgtccagactctagaggaagatggtgggaccgtctcgccctcaaccttcatcaacacctaaagcgattagagcctactataaaatgcattacagaaggacttgcagacccagaagttcgtacaggacaccgactctccttgtaccaaagggcagtgcgattgagagaatcgccctcgtgcaaaaaatttaagcatctattccaacaattacctgaaatggcagttcaagacgtgaaacatgttacaataaccggcagattgtgcccacaacgaggaatgtgcaaatcggtttttgtaatggaagccggcgaggctgcagacccgacaacagtcctctgttcggtcgaggaactagccctcgcacactatcgtcgttccggattcgaccaaggcattcatggagaaggtagtacattttccaccctatatggattactcttatgggatataatcttcatggacggaattccagatgttttccgaaacgcatgccaggcctttcccctagacctatgcactgatagtttcttcacctctagaagaccagctctcgaggctcgccttcaattgattcatgatgcaccagaagaatctctccgagcttgggtagccgctacctggcatgagcaggaaggaagagttgcatccttagtctcgtgggaccgtttcacctctcttcaacaagcacaagacttggtaagttgcctaggaggtccggtcctctctggtgtctgcagacatctagccgccgatttccgtcattgccgtggtggacttcctgatctcgtagtctggaatagtcaatcgcgtcatttcaagttggtcgaggtcaagggtcccaacgatagactcagtcataaacaaatgatttggctcgccgaactacaaaaacttggtgcagaagtagaagtttgccatgtagtcgctgtcggagcaaaatcccaatcgttatcctaa

SEQ ID NO: 29 - DNA, 793 bp, synthetic construct
  source 1..793: mol_type = other DNA
ctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcctcgaccggtcgatctc

SEQ ID NO: 30 - DNA, 120 bp, synthetic construct
  source 1..120: mol_type = other DNA
cgctctgcgcgaggcggctccgccgcggcggagggatacggcgcaccatatatatatcgcggggcgcagactcgcgctccggcagtggtgctgggagtgtcgtggacgccgtgccgttac

SEQ ID NO: 31 - DNA, 441 bp, synthetic construct
  source 1..441: mol_type = other DNA
atggcaaacctggacaagatgctgaacaccaccgtgaccgaggtgcgcaagttcctgcaagcggacagagtgtgcgtgttcaagttcgaggaagattactccggcaccgtctcgcacgaagccgtggacgacagatggattagcatcctgaagacccaggtgcaggacagatacttcatggaaaccagaggcgaggaatacgtccacggcagataccaggccatcgccgacatctacacagccaatctggtcgagtgctacagagacctgctgatcgagtttcaggtgcgggccattctggctgtccccatcctgcaaggcaagaagctgtggggcctgctggtggcccaccaactggccggccctcgggagtggcagacctgggaaatcgacttcctgaaacagcaagccgtggtgatgggcatcgccatccagcagagc

SEQ ID NO: 32 - DNA, 20 bp, synthetic construct
  source 1..20: mol_type = other DNA
  modified_base 1: mod_base = m5c
  modified_base 7: mod_base = m5c
  modified_base 10: mod_base = m5c
  modified_base 12..14: mod_base = m5c
  modified_base 17..19: mod_base = m5c
  modified_base 4: mod_base = m5u
  modified_base 9: mod_base = m5u
  modified_base 15..16: mod_base = m5u
  modified_base 20: mod_base = m5u
caataacatcacccttccct

SEQ ID NO: 33 - DNA, 21 bp, synthetic construct
  source 1..21: mol_type = other DNA
cgcaaaaaacgcaaaaaaaaa

SEQ ID NO: 34 - DNA, 105 bp, synthetic construct
  source 1..105: mol_type = other DNA
gctcctccggcacaacctcccagccaacctcagcagcattatagcgaaggagagttggaggaagatgaagattctgatgacgcgcgcaaaaaacgcaaaaaaaaa

SEQ ID NO: 35 - DNA, 198 bp, synthetic construct
  source 1..198: mol_type = other DNA
atggctcctccggcacaacctcccagccaacctcagcagcattatagcgaaggagagttggaggaagatgaagattctgatgacgcgcgcaaaaaacgcaaaaaaaaatctccctattttagcggaggaggtagtggcggcggcgggtccggcggaggaggcagtggtggcgggagcctcgcgtccaagctgtctcga

SEQ ID NO: 36 - DNA, 90 bp, synthetic construct
  source 1..90: mol_type = other DNA
tctccctattttagcggaggaggtagtggcggcggcgggtccggcggaggaggcagtggtggcgggagcctcgcgtccaagctgtctcga

(19) United States
(12) Patent Application Publication
(10) Pub. No.: US 2025/0032644 A1
(43) Pub. Date: Jan. 30, 2025

(54) COMPOSITIONS AND METHODS FOR TREATING FAN1 ASSOCIATED TRINUCLEOTIDE REPEAT EXPANSION DISORDERS

(71) Applicant: MASSACHUSETTS INSTITUTE OF TECHNOLOGY, Cambridge, MA (US)

(72) Inventors: Christopher Ng, Cambridge, MA (US); David Housman, Cambridge, MA (US)

(21) Appl. No.: 18/784,395

(22) Filed: Jul. 25, 2024

Related U.S. Application Data
(60) Provisional application No. 63/515,383, filed on Jul. 25, 2023.

(51) Int. Cl. A61K 48/00; A61K 38/46; C12N 9/22; C12N 15/10; C12N 15/113

(52) U.S. Cl. CPC A61K 48/0058; A61K 38/465; C12N 9/22; C12N 15/1086; C12N 15/1137; C12Y 301/21; C12N 2310/11

(57) ABSTRACT

Compositions and methods thereof for increasing endogenous FAN1 expression in a target cell are provided. Preferably, compositions include one or more splice switching antisense oligonucleotides targeting FAN1 exon 10-intron 10-exon 11 junction. The compositions are effective in increasing levels of functional FAN1 mRNA transcripts encoding FAN1 derived from the pre-mRNA in a target cell. The compositions and methods are particularly suited for treating, ameliorating, or delaying the onset of a trinucleotide repeat expansion disorder in a subject in need thereof.

### CROSS-REFERENCE TO RELATED APPLICATIONS

This application claims the benefit of and priority to U.S. Provisional Application No. 65/515,383 filed on Jul. 25, 2023, the contents of which is incorporated herein in its entirety.

### REFERENCE TO THE SEQUENCE LISTING

The Sequence Listing submitted as an XML file named “MIT_24829_US_ST26.xml,” created on Jul. 25, 2023, and having a size of 84,843 bytes is hereby incorporated by reference pursuant to 37 C.F.R. § 1.834(c)(1).

### FIELD OF THE INVENTION

This invention is generally in the field of compositions and methods for treating trinucleotide repeat expansion disorders.

### BACKGROUND OF THE INVENTION

FAN1 (Fanconi anemia-associated nuclease 1) has been linked to various human diseases including neurological disorders such as autism, schizophrenia and various trinucleotide repeat expansion disorders (Huntington's disease, cerebrospinal ataxia, fragile X syndrome). In genome-wide association studies, SNPs located within the FAN1 genetic locus have been identified, influencing both the acceleration and delay of the age of onset in Huntington's disease (HD). In the context of seven CAG repeat expansion diseases, including Huntington's disease (HD) and certain spinocerebellar ataxias, age of onset modifications have been associated with particular DNA repair protein variants. Specifically, FAN1 variants exhibit the strongest association with modifying the age of onset in HD. Non-coding disease-delaying FAN1 variants and coding disease-hastening variants (p.R507H and p.R377W) are known, where the former may lead to increased FAN1 levels. Various approaches have been employed to increase FAN1 levels. WO 2022/219353 discloses FAN1 activators used to upregulate the expression of FAN1. However, these methods do not target variants that result in altered FAN1 expression. While WO 2022/219353 discloses the usefulness of FAN1 activators in relation to disease, it lacks comprehensive, translatable methods for enhancing FAN1 expression. Much of the existing data is centered on the effects of reduced FAN1 levels exacerbating HD pathology, or rescue experiments in FAN1 deficient cells. Although some FAN1 overexpression constructs are presented within the context of HD pathology, these limited findings are narrowly applicable to cell model systems. Furthermore, the overexpression constructs designed for transient cell line expression cannot be feasibly extended to other delivery methods such as viral delivery constructs, which could be therapeutically valuable in animal models and patients. Therefore, there is an ongoing need for innovative approaches to increase FAN1 expression, particularly those targeting variants that lead to altered FAN1 splicing and expression.

It is an object of the invention to provide compositions and methods for treating, ameliorating or delaying the onset of a trinucleotide repeat expansion disorder such as myotonic dystrophy (DM), Huntington disease, spinocerebellar ataxia, Friedreich ataxia, and fragile X syndrome in a subject.

It is also another object of the invention to provide safe and efficacious compositions and methods for treating one or more trinucleotide repeat expansion disorders with minimal toxicity.

### SUMMARY OF THE INVENTION

Provided herein are compositions and methods for treating trinucleotide repeat expansion disorders in a subject in need thereof. In some embodiments, the compositions and methods described herein are useful in the treatment of disorders associated with FAN1 activity. The disclosed compositions result in one embodiment to increased levels of FAN1, and other embodiments, in decreased levels of unwanted FAN1 mRNA variants. The disclosed compositions include antisense oligonucleotide sequences (ASOs) that are reverse complements to the FAN1 RNA target sequence of interest. In some forms, the ASO targets dinucleotide repeats of thymine and guanine (TG) in FAN1 (hereinafter, TGASO), which results in decreased FAN1 expression. In some forms the composition increasing FAN1 levels is a splice switching antisense oligonucleotide (SSASO), which targets the FAN1 pre-mRNA, for example, a 3′ cryptic splice site at the 5′ end of a detained intron in the FAN1 pre-mRNA, increasing expression of FAN1. In other embodiments compositions which increase FAN1 levels encodes FAN1, preferably in an AAV vector.

Compositions including one or more splice switching antisense oligonucleotides (SSASOs) in a pharmaceutically acceptable carrier for increasing levels of functional FAN1 mRNA transcripts encoding FAN1 derived from the pre-mRNA (SSASO), are disclosed. Generally, the SSASO includes 8 to 50 nucleotides. In some forms the SSASO specifically hybridizes to a target sequence of the 5′ end of the detained intron within intron 10 of FAN1 pre-mRNA. In some embodiments, the SSASO specifically hybridizes to a target sequence within SEQ ID NO:2. In some embodiments, the target sequence has between about 8 and about 50, inclusive, preferably between about 12 and about 30 contiguous nucleotides of SEQ ID NO:2. In one embodiment, the target sequence for the SSASO has the nucleotide sequence of any one of SEQ ID NOs:4-6. In another embodiment, the SSASO comprises the nucleotide sequence of any one of SEQ ID NOs:7-9.

Splice switching antisense oligonucleotides (SSASOs) including between about 12 and 50 nucleotides are also provided. The SSASO specifically hybridize to a target sequence of the 5′ end of the detained intron within intron 10 of FAN1 pre-mRNA are also described. The SSASO specifically hybridizes to a target sequence within SEQ ID NO:2. In some embodiments, the target sequence has between about 8 and about 50, inclusive, preferably between about 12 and about 30 contiguous nucleotides of SEQ ID NO:2. In one embodiment, the target sequence has the nucleotide sequence of any one of SEQ ID NOs:4-6. In another embodiment, the splice switching ASO comprises the nucleotide sequence of any one of SEQ ID NOs:7-9. In preferred embodiments, the splice switching ASO is chemically modified, for example, via phosphorothiate (PS) backbone modification, and/or modified at the 2′ sugar position selected from the group consisting of 2′-O-methyl (2′-OMe) and 2′-O-methoxyethyl (2′-MOE). In preferred embodiments, the splice switching ASO is administered in an amount effective to increase the level of mRNA and/or protein expression of FAN1 in one or more neuronal cells compared to the level prior to the administration of the splice switching ASO.

Methods of ameliorating or delaying the onset of a trinucleotide repeat expansion disorder in a subject in need thereof are provided. Typically, methods effectively increase the level of mRNA and/or protein expression of FAN1. In some forms, the methods decrease the levels of alternatively spliced nonfunctional FAN1. In some embodiments, the subject has more than 17 dinucleotide repeats of thymine and guanine (TG) in intron 10 of the FAN1 pre-mRNA, for example 18, 19, 20, 21, 22, 23, or more than 23 dinucleotide TG repeats in intron 10 of the FAN1 pre-mRNA. In other embodiments, the subject has a reduced level of mRNA and/or protein expression of FAN1 compared to a healthy control. In preferred embodiments, the subject has a reduced efficiency of splicing in exon10 and exon11 junction in the pre-mRNA of FAN1 to produce functional mRNA transcripts of FAN1.

Generally, in some forms, the methods involve the step of administering to the subject a composition effective to increase levels of functional FAN1 mRNA transcripts encoding FAN1 derived from the pre-mRNA, for example, using one or more SSASOs, or a Cas enzyme and a single guide RNA (sgRNA) sequence, in a pharmaceutically acceptable carrier. In some embodiments, the SSASOs specifically target a 3′ cryptic splice site in intron 10 of FAN1 mRNA, or the 5′ end of the detained intron within intron 10 of FAN1 mRNA. Preferably, the SSASOs specifically target SEQ ID NO:2, or a region of between about 8 and about 50, inclusive, preferably between about 12 and about 30 contiguous nucleotides of SEQ ID NO:2.

In one embodiment, the splice switching ASO has the nucleotide sequence of any one of SEQ ID NOs: 7-9. Chemical modifications have improved oligonucleotide binding affinity, stability and pharmacodynamic properties. Thus, in some embodiments, the SSASO is chemically modified, for example, via phosphorothiate (PS) backbone modification, and/or modified at the 2′ sugar position selected from the group consisting of 2′-O-methyl (2′-OMe) and 2′-O-methoxyethyl (2′-MOE). In preferred embodiments, the splice switching ASO is administered in an amount effective to increase the level of mRNA and/or protein expression of FAN1 in one or more neuronal cells compared to the level prior to the administration of the splice switching ASO. In other embodiments, the step of increasing the level of mRNA and/or protein expression of FAN1 is achieved by CRISPR/Cas based gene editing, for example, using Cas 9, Cas13, C and an sgRNA/crRNA guide.

The methods are suited for ameliorating or delaying the onset of trinucleotide repeat expansion disorder including Huntington's disease, cerebrospinal ataxia, or fragile X syndrome. In one embodiment, the trinucleotide repeat expansion disorder is Huntington's disease. In a preferred embodiment, the increase in the level of mRNA and/or protein expression of FAN1 or decrease in the levels of alternatively spliced nonfunctional FAN1 is effective to delay the onset of one or more symptoms associated with Huntington's disease.

Methods of ameliorating or delaying the onset of a trinucleotide repeat expansion disorder in a subject in need thereof by administering to the subject one or more SSASOs or TGASOs in a pharmaceutically acceptable carrier are also provided. In some forms methods are effective to increase levels of functional FAN1 mRNA transcripts encoding FAN1 derived from the pre-mRNA. In some embodiments, the SSASO specifically target the detained intron within intron 10 of FAN1 mRNA, or the 5′ end of the detained intron within intron 10 of FAN1 mRNA. Preferably, the SSASOs specifically target SEQ ID NO:2, or a region of between about 8 and about 50, inclusive, preferably between about 12 and about 30 contiguous nucleotides of SEQ ID NO:2. In one embodiment, the SSASO has the nucleotide sequence of any one of SEQ ID NOs:7-9. In preferred embodiments, the SSASO is chemically modified, for example, via phosphorothiate (PS) backbone modification, and/or modified at the 2′ sugar position selected from the group consisting of 2′-O-methyl (2′-OMe) and 2′-O-methoxyethyl (2′-MOE). In preferred embodiments, the splice switching ASO is administered in an amount effective to increase the level of mRNA and/or protein expression of FAN1 in one or more neuronal cells compared to the level prior to the administration of the SSASO.

Also disclosed is a minigene reporter system including the FAN1 exon 10-intron 10-exon 11 junction that contains the variable TG repeat, and a reporter gene. In some embodiments, the nucleotide sequence of exon 10-intron 10-exon 11 junction of FAN1 has the nucleotide sequence of SEQ ID NOs:1-3. In embodiment, the reporter gene encodes enhanced green fluorescent protein. The disclosed minigene EGFP reporter system can be used in the high-throughput screening of small molecules and splicing modulators that modulate FAN1 expression in the treatment of disorders associated with FAN1 activity. Accordingly, methods of identifying one or more splice modulators that can increase levels of functional FAN1 mRNA transcripts encoding FAN1 derived from the pre-mRNA in one or more neuronal cells including i) contacting the minigene reporter system and one or more candidate molecules, and ii) determine the level of reporter gene expression, and

iii) select the candidate molecules that increase the level of reporter gene expression as suitable for increasing the level of mRNA and/or protein expression of FAN1 in one or more neuronal cells.

### BRIEF DESCRIPTION OF THE DRAWINGS

FIGS. 1A-1B are graphs depicting modifiers that delay HD age of onset in the FAN1 locus are brain-specific expression QTLs. FIG. 1A is a graph of eQTL data from the GTeX database indicating that patients with the rs3512 variant have increased gene expression of FAN1 in cortex tissue (p=7.8e-8), showing Rank Normalized Gene Expression for each of Homogenous Reference (N=82); Heterogenous (N=49); and Homogenous Alt (N=5). FIG. 1B is a bar graph of Alternate allele/reference allele (0-7) for each of 6 patients (214, 553, 5043, 5049, 5079 and 5092, respectively), showing that FAN1 allelic imbalance test confirms higher expression of variant allele in heterozygous Venezuelan patient brains. RNA was isolated from BA9 prefrontal cortical tissue of HD patients heterozygous for the rs3512 variant and cDNA was reverse transcribed. PCR was performed with specific primers around the rs3512 variant, and the subsequent PCR products were sequenced. Allelic imbalance of FAN1 expression was then quantified by read counts of the reference and variant alleles.

FIGS. 2A-2D. A variable dinucleotide repeat is present at the 3′ splice site of FAN1 intron 10 and likely in linkage disequilibrium with HD onset delaying variant. FIG. 2A shows whole genome sequencing data from Venezuelan HD patients suggesting that shorter TG dinucleotide repeats may be in linkage disequilibrium with the 3′ UTR variant (rs3512) in FAN1 that delays HD onset. Patients from the HD Venezuelan Kindreds were genotyped by whole genome sequencing. Pairwise table shows overlap of patients identified with various TG repeat lengths in the FAN1 intron10 and patients identified with the rs3512 variant. A venn diagrams shows overlap of patients identified with the short TG repeat lengths (17TG, 18TG) in the FAN1 intron10 and patients identified with the rs3512 variant. FIG. 2B is a bar graph of Residual HD age of onset over Genotype for each of 8 genotypes (TG22, TG23, TG20, TG19, TG21, TG17, TG18, and rs3512, respectively), that shows residual age of motor onset in HD patients as calculated by subtracting the observed clinical age of onset with the age of onset expected by CAG repeat length (log-linear regression). For each group of patients identified with a particular TG repeat length, the distribution of age of motor onset was plotted as a bar graph of the mean+/−standard deviation. FIGS. 2C and 2D show the putative secondary structure of the dinucleotide variable repeat at the 3′splice site of FAN1 intron 10, formed from a nucleic acid sequence of:

UUCUGAAUCUAAAAUAUUUCUAUUAUUUUCUUGAAAUUGAGUGUGCAGUA
GGUUAUGGUGGUGUUUUGGAAGAAACAGAUAAAACAGAUUUUGUGUGUGU
GUGUGUGUGUGUGUGUGUGUGUGUGACCUUGUCUUAGGGAUUCAUGGCGA
(SEQ ID NO: 24; having 17 “TG” repeat sequences)
(FIG. 2C);

(SEQ ID NO:24; having 17 “TG” repeat sequences) (FIG. 2C); and

UUCUGAAUCUAAAAUAUUUCUAUUAUUUUCUUGAAAUUGAGUGUGCAGUA
GGUUAUGGUGGUGUUUUGGAAGAAACAGAUAAAACAGAUUUUGUGUGUGU
GUGUGUGUGUGUGUGUGUGUGUGUGUGUGUGUGUGACCUUGUCUUAGGGA
UUCAUGGCGA
(SEQ ID NO: 25; having 22 “TG” repeat sequences)
(FIG. 2D),

A (SEQ ID NO:25; having 22 “TG” repeat sequences) (FIG. 2D), respectively.

FIG. 3 is a schematic overview showing approaches to investigate FAN intron 10 splicing/expression patterns in vitro using the SHSY5Y neuroblastoma cell line.

FIGS. 4A-4H. Targeting of key splicing elements of the FAN1 intron10 with ASO treatments in SHSY5Y neuroblastoma cells results in significant changes in FAN1 splicing and expression (See Materials and Methods). FIG. 4A is a schematic of the domain structure of the FAN1 pre-mRNA, showing exon10, the detained intron, and exon 11, and indicating the site of multiple TG repeats (TG_{N}) and the relative location of ASO16 and ASO12. FIGS. 4B-4C are bar graphs of FAN1 normalized gene expression for each of control (nt), ASO12, ASO13 and ASO14, ASO15, ASO16 and (ASO12+ASO16), respectively, for each of exon 8 spanning qPCR (FIG. 4B) and exon 10 spanning qPCR (FIG. 4C), respectively, showing ASOs targeting the 5′ end of the detained intron increases FAN1 expression in SHSY5Y cells. FIG. 4D is a schematic of the domain structure of the FAN1 pre-mRNA, showing exon10, the detained intron, and exon 11, and indicating the site of multiple TG repeats (TGN) and the relative location of ASO16, 17, 18 and 19. FIGS. 4E-4F are bar graphs of FAN1 normalized gene expression for each of control (nt), and ASO17, respectively, for each of exon 8 spanning qPCR (FIG. 4E) and exon 10 spanning qPCR (FIG. 4F), respectively, showing refinement of ASOs targeting the 5′ end of the detained intron leads to further increases FAN1 expression in SHSY5Y cells. FIG. 4G is a schematic of the domain structure of the FAN1 pre-mRNA, showing exon10, the detained intron, and exon 11, and indicating the site of multiple TG repeats (TGN) and the relative location of ASO16, 17, and ASO3. FIG. 4H is an image of a western blot showing each of FAN1 expression and GAPDH expression with ASOntc, ASO16, ASO17, and ASO3, respectively. ASO17 targeting the 5′ end of the detained intron increases FAN1 expression in SHSY5Y cells at the protein level.

FIGS. 5A-5G. A minigene reporter system of the FAN1 exon10-intron10-exon11 splice junction evaluates efficacy of ASO treatments and influence of TG repeat lengths on FAN1 splicing. FIG. 5A is a schematic depicting a minigene reporter system of the expression of FAN1 intron10. The exon10-intron10-exon11 splice junction of FAN1 (intron10 is truncated to contain the key splicing elements) is placed before a d1EGFP reporter. The TG repeats is engineered to include an additional 6 TG repeats (TGN+6), and includes a stop codon (indicated as [symbol] ). EGFP is frameshifted from the promoter such that proper protein expression and subsequent fluorescence is only achieved when the FAN1 intron10 is spliced out. An mPlum fluorescent reporter is co-expressed on an independent promoter as a transfection control. The relative locations of the ASO17 biding sequence is depicted, as well as the resulting functional exon10-exon11-d1EGFP expressed product. FIG. 5B is a graph of splicing index (GFP+; 0-100) over TG repeat length (0-50), of data from flow cytometry of the FAN1 exon10-exon11 junction. The data confirm decreased splicing efficiency as the length of TG repeats increase. FIG. 5C is a graph of splicing % (0-100) over TG repeat length (0-50), of data from Qpcr of the FAN1 exon10-exon11 junction. The data confirm decreased splicing efficiency as the length of TG repeats increase. FIG. 5D is a bar graph of % splicing (normalized GFP+ cells) for each of control, aso17 and aso3. The data shows flow cytometry of altered splicing efficiency in the FAN1 exon10-exon11 junction when SHSY5Y cells are treated simultaneously with ASO treatments. FIG. 5E is a graph of Fold change expression (0-4.5) for each of control, aso17 and aso3. The data shows Qpcr of altered splicing efficiency in the FAN1 exon10-exon11 junction when SHSY5Y cells are treated simultaneously with ASO treatments. FIG. 5F is a schematic of a plasmid map of a minigene reporter system of FAN1 intron10. The exon10-intron10-exon11 splice junction of FAN1 (intron10 is truncated to contain the key splicing elements) is placed before a d1EGFP reporter. EGFP is frameshifted from the promoter such that proper protein expression and subsequent fluorescence is only achieved when the FAN1 intron10 is spliced out. An mPlum fluorescent reporter is co-expressed on an independent promoter as a transfection control. FIG. 5G is a schematic work flow showing a methodology for the use of the described FAN1 splicing reporter minigene system to high-throughput screen small molecule drug libraries.

FIGS. 6A-6E are cartoons showing efficient packaging of FAN1 transgene for FAN1 overexpression. AAV expression cassette developed as disclosed herein in comparison to traditional AAV cassette. FIG. 6A shows a FAN1 cDNA flanked by AAV ITRs. FIG. 6B shows an expression cassette including a CAG promoter, a GFP gene, a FAN1cDNA, WPRE PA 3′UTR, flanked by AAV ITRs. FIG. 6C shows an expression cassette including a core EF1a promoter, an iRFP670nano gene, a FAN1cDNA, and WSL 3′UTR, flanked by AAV ITRs. Overexpression of FAN1 by AAV is achieved by efficient packaging of the large 3.1 Kb transgene into an AAV cassette containing alternative ITR elements, a shortened 3′UTR poly-adenylation site and signal sequence, miniature transcriptional promoter (core EF1a/core EF1a+HTLV intron), and a small near-infrared (NIR) fluorescent protein tag at the N-terminus. FIG. 6D is a cartoon illustration of a work flow for the overexpression of FAN1 by AAV.PhP.eB by retroorbital injection in the R6/1 mice model of HD, achieved by efficient packaging of the large transgene. FIG. 6E is an image of a Western blot of cortical lysates that confirms FAN1 overexpression in mice using AAV.PhP.Eb viral delivery. A combination of a miniature transcriptional promoter core EF1a, shortened 3′UTR W3SL, and miRFP670nano N-terminal tag is able to successfully overexpress FAN1 in mice. A human Synapsin promoter combined a short HA N-terminal tag does not overexpress FAN1 in mice. FIG. 6F is a schematic representation of a plasmid map, depicting each of the components of the construct of FIG. 6C.

FIGS. 7A-7B are representative examples of PeakScanner raw data from the striatum of one control (FIG. 7A) and one FAN1-treated R6/1 HD mouse (FIG. 7B) at 30 weeks of age are shown. FIG. 7C is a graph showing the instability index calculated in striatum, cortex, and liver of FAN1-treated and control miRFP670-treated R6/1 mice. FIG. 7D-7F are images of electron microscopy, depicting structures of cells in samples of each of wild type (FIG. 7D), R6/1 HD (FIG. 7E), and R6/1 HD+FAN1 (FIG. 7F) mice, respectively. The position and structure of the nucleus is indicated (Nu), where present. The images show potential rescue of structural pathology in R6/1 mice treated with FAN1 overexpression by PhP AAV.

FIGS. 8A-8B are bar graphs depicting modelling of cellular and organelle-based effects of the described systems for increased expression of FAN1 in each of wild-type, HD and HD+FAN1 groups, respectively. FIG. 8A shows percent volume over intracellular void/swelling for each group, respectively; FIG. 8B shows percentage normalized for wild-type for mitochondrial volume and synapse density volume for each group, respectively. Data are from the semi-automated annotation of subcellular structure with Dragonfly software quantifies the restoration of structural integrity, mitochondrial density, and synaptic density in FAN1 overexpressing R6/1 mouse tissue.

FIG. 9A-9C show the design strategy of ASOs targeting the TG repeat and decreased FAN1 expression in SHSY5Y cells in response to ASO3, ASO5 and ASO6. FIG. 9A is a schematic of the FAN 1 pre-mRNA, with the variable TG dinucleotide repeat that is present at the 3′ splice site of FAN1 intron 10-exon 11 splicing junction, depicting the nucleic acid sequence of the junction region that is targeted by each of ASO6, ASO5 and ASO3, respectively. FIGS. 9B and 9C are graphs showing qPCR data for normalized expression of FAN1 for each of control (nt), ASO3, ASO5 and ASO6, respectively, using qPCR based on spanning exon 8 (FIG. 9B) and exon 10 (FIG. 9C), respectively.

FIGS. 10A-10C depict successful codon optimization of the FAN1 sequence for enhancing expression of FAN1 protein. FIG. 10A is an image of a Western blot analysis, showing expression of each of FAN1 and ACTB in each of untreated, vector only, vector+FAN1 (3×10e11), and vector+FAN1(3×10e12) groups, respectively. FIG. 10B is an image of a Western blot analysis, showing expression of each of FAN1 and ACTB in each of codon opt FAN1, wild-type groups, respectively. FIG. 10C is a bar graph showing relative FAN1 protein expression (0-6) for each of wild-type and codon optimized FAN1, respectively. The data confirm increased FAN1 levels in cortical lysates of mice administered with the optimized AAV vector and further codon optimization of the FAN1 cDNA.

FIGS. 11A-11C are Schematics depicting each of two AAV constructs. FIG. 11A is a control vector with core EF1a promoter. FIG. 11B is a MHCK7-Myospreader construct optimized for muscle-specific expression. FAN1-delivering myoAAVs with either EF1a or MHCK7-Myospreader modifications are systemically administered to wild-type mice to evaluate muscle tissue targeting. FIG. 11C is an image of Western blot analysis, showing expression of each of FAN1 and S6 Ribo, as expressed using each of EFa1-miRF67nano (control), EFa1-miRF67nano-FAN1, and MHCK7-Myospacer FAN1 constructs, respectively. FAN1 protein expression levels in quadriceps tissue post-MyoAAV injection, demonstrating the vastly improved expression with MHCK7-Myospreader construct over the control EF1a vectors.

FIG. 12 is a bar graph showing FAN1 gene expression (2ΔCp) (0-4) for each of gapmer-NTC, and TDP-43 KG groups, respectively.

### DETAILED DESCRIPTION OF THE INVENTION

The disclosed compositions and methods are based at least on the discovery of a variable TG dinucleotide repeat in intron 10 encoding section of the FAN1 gene, as a function link to modification of repeat expansion disorder patient outcomes; longer repeats result in inefficient splicing of the FAN1 exon10-exon11 junction, decreasing FAN1 levels.

### I. Definitions

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

The term “dosage regime” refers to drug administration regarding formulation, route of administration, drug dose, dosing interval and treatment duration.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination.

The term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt”, as used herein, refers to derivatives of the compounds defined herein, wherein the parent compound is modified by making acid or base salts thereof. Lists of suitable salts are found in Remington's Pharmaceutical Sciences, 20th ed., Lippincott Williams & Wilkins, Baltimore, MD, 2000, p. 704; and “Handbook of Pharmaceutical Salts: Properties, Selection, and Use,” P. Heinrich Stahl and Camille G. Wermuth, Eds., Wiley-VCH, Weinheim, 2002.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with one or more eating disorders are mitigated or eliminated, including, but are not limited to, decreasing symptoms resulting from the disease, increasing the quality of life of those suffering from the disease, decreasing the dose of other medications required to treat the disease, delaying the progression of the disease, and/or prolonging survival of individuals.

The term “biodegradable”, generally refers to a material that will degrade or erode under physiologic conditions to smaller units or chemical species that are capable of being metabolized, eliminated, or excreted by the subject. The degradation time is a function of composition and morphology.

### II. Compositions

Compositions for increasing FAN1expression are disclosed, which use a different approach than previously disclosed. For example, WO 2023/009396 contemplates increasing FAN1 expression by diverting mRNAs for isoform 2 (533 aa) which is non-functional, to isoform 1 during RNA processing, based on the observation that mRNA isoforms coding for the shorter protein isoform are generated by alternative polyadenylation, not by alternative splicing. To this end, WO 2023/009396 discloses SSASO that potentially modulate polyadenylation of FAN1 and effectively, the full length protein.

The disclosed compositions and methods are based at least on the discovery that increase in endogenous FAN1 expression at both the RNA and protein level can be achieved by targeting the 5′ end of the detained intron within intron 10 of FAN1, for example, using splice switching antisense oligonucleotide (SSASO), as shown in Examples. In some forms, the disclosed compositions result in decreased levels of alternatively spliced nonfunctional FAN1 (FAN1 with TG repeats as disclosed herein).

Accordingly, compositions for increasing endogenous FAN1 expression in a target cell are provided. In some embodiments, compositions include one or more SSASO. In other embodiments, compositions include CRISPR/Cas for editing mRNA of FAN1. In some forms, compositions for decreasing alternatively spliced nonfunctional FAN1 are provided. The compositions include one or more TGASOs.

### A. Compositions for Increasing FAN1 Expression

i. Splice Switching Antisense Oligonucleotides

Splice-switching oligonucleotides (SSASOs) are short, synthetic, antisense, modified nucleic acids that base-pair with a pre-mRNA and disrupt the normal splicing repertoire of the transcript by blocking the RNA-RNA base-pairing or protein-RNA binding interactions that occur between components of the splicing machinery and the pre-mRNA. Splicing of pre-mRNA is required for the proper expression of the vast majority of protein-coding genes, and thus, targeting the process offers a means to manipulate protein production from a gene.

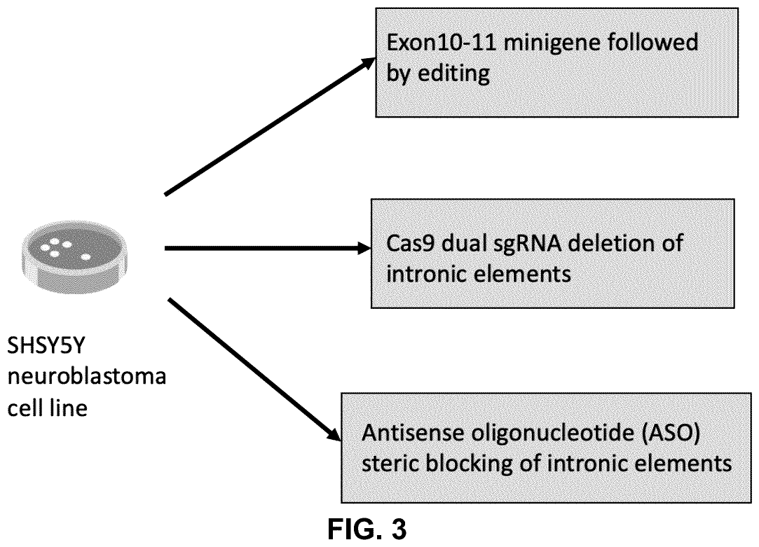

Splicing modulation is particularly valuable in cases of disease caused by mutations that lead to disruption of normal splicing or when interfering with the normal splicing process of a gene transcript may be therapeutic. Most protein-coding genes are comprised of coding sequences that are interspersed with non-coding sequences. Following gene transcription, these intervening, non-coding RNA sequences, called introns, are removed and the coding RNA sequences, called exons, are ligated together in a process called pre-mRNA splicing. This splicing gives rise to the final mRNA that is translated into a protein. Pre-mRNA splicing requires precision and accuracy in order to ensure that the proper open reading frame is maintained for efficacious protein production during translation. This high fidelity is achieved, in large part, by sequences and structures within the RNA transcript that direct the binding of splicing proteins that aid in positioning the RNA in a manner that facilitates the correct cleavage and ligation reactions of splicing. These cleavage reactions occur at conserved sequences called the 5′ splice site at the 5′ end of an intron and the 3′ splice site at the 3′ end of an intron. The splice sites are recognized through interactions with a multi-megadalton ribonucleoprotein complex called the spliceosome. (Reviewed in Havens, et al., Nucleic Acids Res, 44(14):6549-6563 (2016). The disclosed methods in some forms use of short antisense oligonucleotides (ASOs) that base-pair in an antisense orientation to a specific pre-mRNA sequence and, in so doing, modulate splicing by interfering with the normal protein:RNA or RNA:RNA interactions that direct splicing. ASOs that specifically target splicing are referred to here as splice-switching antisense oligonucleotides (SSOs). SSASOs are ASOs designed to base-pair and create a steric block to the binding of splicing factors to the pre-mRNA. In this way, SSASO base-pairing to a target RNA alters the recognition of splice sites by the spliceosome, which leads to an alteration of normal splicing of the targeted transcript. FIG. 3.

In some embodiments, compositions include one or more splice switching antisense oligonucleotides, preferably targeting a region within exon10-intron10-exon11 junction of FAN1.

Nucleotide sequences of exon10, intron10, and exon11 of FAN1 are provided below.

SEQ ID NO:1 is the nucleotide sequence of exon10 of FAN1.

(SEQ ID NO: 1)
GTGACCATCACAGGCAGGCTGTGCCCACAGCGTGGGATGTGCAAGTCTG
TGTTTGTGATGGAGGCCGGGGAGGCCGCTGACCCCACCACGGTCCTGTG
CTCTGTGGAGGAGCTGGCACTGGCCCATTACAGACGCAGCGGTTTTGAC
CAGG.

SEQ ID NO:2 is the nucleotide sequence of intron10 of FAN1.

(SEQ ID NO: 2)
GTAACTGAGCAGGCTTTCTCTTGTGGCACCCAGCCCCGGGTGGACGAGC
AGCAGCACTGGATGGGCTGTCAGCAGTGGGCTGCTGTCCTCTCTCTGTC
CTCTGCTCACAGTGGAAGATGCTGTGGGCTGGGGGATGTCTTCCTATTC
TTCCCCTTATCAATGATAAGGAGTAGGTCAGGGGTAGTTGTCCCACAGG
GCCTGGCTCTGATCTTTTTTCTTTTATAAAAACTTATAAAAGTTATGGA
AGGTACTGCACAAGCCTTTGTACAGAAGTCAGGGGGACAGTGTGCTCAA
CCCCACGGACACCCACTTCCATTCAGTGTTCCTTCTGTTCTCTCCTGCC
CCTTCCCTGGGATTTTAGAAGCTGGCCCCAGATATCCTCTCAGGGGCCC
TGCGGCCCATGCTAGTCGCTTTCGGGAACAGCCACTGAGCCTTCCGCCC
TTGGGCCTGGCAGGACTCTCCCTGTGCCCGGGCTGCAGGGTTACCAGTG
TTAAAAACACTGATATTTTCAGATGTGTGAAAATCAGGACATGATTTCA
GTATTCCCCGAGTGTTCGGAAGATTAACAAGTACTATAGAAAGACAACC
ACAAGATAGGAGTTTACAAAGCCCAGGTTCTTTCTGATGAGTGTTGAGA
TTCATCTACATAATATCTTTATTACTTACAGGGAAGGGTGATGTTATTG
GGGAAGACGTGCAAATGCCAGTGAAGACAGAGAGATACAGTAGGCCTGA
AATGGTTAGAAATAGAAGAATAGAATGCACATTCAAGGCCCCGAGAGAC
GTGGAAACTGGACACTCGCTGGAGGAGGAAGCAGGCTGGGACGTGGGAG
CGCTGAAATGCTTTCAGTTGAGCCATGAGCCTGAAAAACACACGATTCC
TTTCCCAGAGTAGAAACTATTTTTCCCTGATTAAAATCGATGCCGTGAA
ACTGGGCTGAACTCTGGCTGCCAGGCTTTATGCCACAGGGAATTTTGTG
TCAGAGCGATCTCAACCTCTTCTGGGAGGTGATTTCTGAAGTACTGCAC
CAAAAATGATTTCACTTATAACACAAATGCACAGCATGGACCACTTAGG
AGTTAAGGAACGAACCAGGCACGGTAGCTTACACTTGTAATCCCAGCAC
TTGGGGAGGCTGAGACAGGAGGATCACTTGAACTCTGAAGACGGAGGTG
GGAGGATCTCTTGACCCCAGGAGGTCAAGGCTGCAGTGAGCCAAGATTG
TGCCACTGCACTCCAGTCTGGGTGACAGAGTGAGACCCTGTCTCAAAAC
AAAACAAACAAAGGAGTTCAGAAATGATCTGGCCTTTATATTCCTGCCT
GATCGTCAGTGTATTCACCAGGACGGAACACTGACTGGAAATCAGGTCC
TAGCTCCACTCCTGGCTGGGAAAGAGCATGAAGTGTTCTAGGAAGAAAA
GTCCTCCTGGAAGACTTGGCAACAGCCAGGAGTCCTGAGTGAACCGCTG
TCCTTTTGAATCCCTGAGACGGGCTGCAGGGATGAGGGGCTAGAACTGA
CAGGAAGACAGGACAGAGAGCAGCACAGCCTCAGAAGTGCAGGACAGAG
GTGACCCAGGCAGGCATGACGGGCTGTGGCTTCCTGCCCTCTGCTCTCA
CAGCACCCCTGATCCCACTCACTGTGGTACCACGCAGATGGGTTGGGGC
CTGTAAATGTCAGGTGGGACCAGCCAGAGGAGCTTTGCTGCCAGTACTG
GCATGTCAGGATGGGGGTCTGGTGGTCAGCACCTCCTCTGGCCAGTGGT
CTTGTGGGGAGAGGCATCCATGTGGCCTCCTCCTCTGTCGGGAGGGCTG
ATGTGCAGAGAAGTACGGACTTGGAGGGCAGCTGGATGAGCTGGGGCTT
GCTCAGGGACACAGGTCTGTGTGTTGTGCCAAGGCTGACCCCCTCATCC
CACACCAGACCCTGCCTCTGACTCTGTGACCTCAGCACCTGACTTCTGT
CTCTCAGGTCATCTCCCAGTCAGTAAAACATTTGTGTGACGTGAGGAGG
GGCACTGAAGTGGGTGATCTTTAAGGCCAGATGTCTCTGTAAAAGCCTT
GACTATCGGAAGCACTGGGCTAGGGCAGTGTAGTACCCAGGGGGATGAG
GTGCATCAGAGCAGCACCTGCTGAGGCCAAGAACGGAGGTGGCTGCTGC
CGGCCTCCGGGAGGTCCCCTTAGGGAAGCATTTCTTCAGGGGTGGCCCA
GGGACCTCCGGCATCAGGGCCTGCATGGGGATTCTGTGCCAGCCCAGCC
CTGCTAAGTCCCAGCCTTGGGAACCTGAGGAGGGTATCTGCCTATTTTT
CTTTTAAACATGTTTCTTAGGTTATTCACTAAAGTTTGAGAACCACTGC
TTTGTGGCTTTGAAATGTTCATTTGAATTTTTCTATGATTACTAAGATT
TTTGCCCTTAAGGCTCTGTATATAAACAACATACTGTATAAAATAAACT
GGGAATGGCGTGAAAACAGCATTTGCTTCTGAATCTAAAATATTTCTAT
TATTTTCTTGAAATTGAGTGTGCAGTAGGTTATGGTGGTGTTTTGGAAG
AAACAGATAAAACAGATTTTGTGTGTGTGTGTGTGTGTGTGTGTGTGTG
TGTGACCTTGTCTTAG

(SEQ ID NO:2) (TG repeat is in bold font).

SEQ ID NO:3 is the nucleotide sequence of exon11 of FAN1.

(SEQ ID NO: 3)
GGATTCATGGCGAAGGGTCCACCTTCAGCACCCTGTATGGCCTCCTCCT
GTGGGACATCATCTTCATGGATGGGATTCCGGATGTCTTCAGAAACGCC
TGTCAGGTACTCCAGTGCCCCTGCCCCACGAGTAGGTC

In some embodiments, the SSASOs specifically target the detained intron within intron 10 of FAN1 mRNA. In preferred embodiments, the splice switching ASOs specifically target the 5′ end of the detained intron within intron 10 of FAN1 mRNA.

In some embodiments, the SSASO specifically targets a nucleotide sequence within the detained intron 10 of FAN1 mRNA as set forth in any one of SEQ ID NOs:4-6:

(SEQ ID NO. 4)
TTACTTACAGGGAAGGGTG,
(SEQ ID NO. 5)
GGTGATGTTATTGGGGAAG,
and
(SEQ ID NO. 6)
TTTATTACTTACAGGGAAGGG.

In one embodiment, the splice switching ASO specifically targets the nucleotide sequence of SEQ ID NO: 4 within intron 10 of FAN1.

### Exemplary SSASOs

In some embodiments, the SSASOs include about 12 to 50 nucleotides, and specifically hybridize with a segment of the 5′ end of the detained intron within intron 10 of FAN1 mRNA. In preferred embodiments, the splice switching ASOs specifically hybridize with a segment of nucleotides in SEQ ID NO. 2, or SEQ ID NOs. 4-6.

In some embodiments, the SSASO specifically target the 5′ end of the detained intron within intron 10 of FAN1 mRNA has the nucleotide sequence of any one of SEQ ID NOs:7-9 or 32 and increases FAN1 expression/solicing). Exemplary SSASO sequences include:

CAATAACATCACCCTTCCCT

(SEQ ID NO:32, “ASO16”, where T is a 5-methyl Uridine nucleotide and C is a 5-methyl Cytidine nucleotide),

CACCCTTCCCTGTAAGTAA

(SEQ ID NO:7, “ASO17 where T is a 5-methyl Uridine nucleotide and C is a 5-methyl Cytidine nucleotide),

CTTCCCCAATAACATCACC

(SEQ ID NO:8, “ASO18 where T is a 5-methyl Uridine nucleotide and C is a 5-methyl Cytidine nucleotide), and

CCCTTCCCTGTAAGTAATAAA

SEQ ID NO:9, “ASO19”, where T is a 5-methyl Uridine nucleotide and C is a 5-methyl Cytidine nucleotide).

In a preferred embodiment, the SSASO specifically target the 5′ end of the detained intron within intron 10 of FAN1 mRNA has the nucleotide sequence of SEQ ID NO:7.

In some embodiments, one of more SSASOs that can decrease the expression of FAN1 has the nucleotide sequence of any one of SEQ ID NOs:7-9.

ii. AAV Overexpressing FAN1

Recombinant adeno-associated viruses (AAVs) are commonly used vehicles for in vivo gene transfer and promising vectors for therapeutic applications. However, overexpression of FAN1 has been difficult to accomplish thus far due to the behavior of the large FAN1 transgene, in vivo.

In some forms, endogenous levels of FAN1 can be increased by overexpression of FAN1, preferably using the disclosed AAV system. Overexpression of FAN1 by AAV can be accomplished as disclosed herein by efficient packaging of the large 3.1 Kb transgene into an AAV cassette containing alternative ITR elements, a shortened 3′UTR poly-adenylation site and signal sequence, miniature transcriptional promoter (core EF1a/core EF1a+HTLV intron), and a small near-infrared (NIR) fluorescent protein tag at the N-terminus.

Thus, in some forms, the AAV can be: i) FAN1 overexpressing with core EF1a promoter and miRFP670nano fluorescent N-terminal tag.

An exemplary sequence for a miRFP670nano fluorescent N-terminal tag is:

(SEQ ID NO: 15)
ATGGCAAACCTGGACAAGATGCTGAATACCACAGTAACAGAGGTGCGGC
AGTTCCTGCAGGTGGACAGAGTGTGCGTGTTCCAGTTTGAGGAGGATTA
TAGCGGAGTGGTGGTGGTGGAGGCCGTGGACGATAGGTGGATCTCCATC
CTGAAGACCCAGGTGCGGGATAGATACTTCATGGAGACAAGGGGCGAGG
AGTATTCTCACGGCCGCTACCAGGCCATCGCCGACATCTACACCGCAAA
CCTGACAGAGTGCTACAGGGATCTGCTGACACAGTTTCAGGTGAGAGCA
ATCCTGGCCGTGCCCATCCTGCAGGGCAAGAAGCTGTGGGGCCTGTTGG
TGGCACACCAGCTGGCGGCCCCTAGACAGTGGCAGACCTGGGAGATCGA
CTTTCTGAAGCAGCAGGCCGTGGTGGTGGGCATCGCCATCCAGCAGAGC
GGAGGTGGTGGATCAGGAGGAGGTGGTTCTGGAGGTGGTGGATCAGGAG
GAAGATCTCGAGCT.

In other forms, the fluorescent tag is miRFP670nano3—a brighter fluorescent tag evolved from miRFP670nano. An exemplary sequence for a miRFP670nano3 fluorescent N-terminal tag is:

(SEQ ID NO: 31)
ATGGCAAACCTGGACAAGATGCTGAACACCACCGTGACCGAGGTGCGCA
AGTTCCTGCAAGCGGACAGAGTGTGCGTGTTCAAGTTCGAGGAAGATTA
CTCCGGCACCGTCTCGCACGAAGCCGTGGACGACAGATGGATTAGCATC
CTGAAGACCCAGGTGCAGGACAGATACTTCATGGAAACCAGAGGCGAGG
AATACGTCCACGGCAGATACCAGGCCATCGCCGACATCTACACAGCCAA
TCTGGTCGAGTGCTACAGAGACCTGCTGATCGAGTTTCAGGTGCGGGCC
ATTCTGGCTGTCCCCATCCTGCAAGGCAAGAAGCTGTGGGGCCTGCTGG
TGGCCCACCAACTGGCCGGCCCTCGGGAGTGGCAGACCTGGGAAATCGA
CTTCCTGAAACAGCAAGCCGTGGTGATGGGCATCGCCATCCAGCAGAG
C.

ii) FAN1 overexpressing with EF1a HTLV promoter and miRFP670nano fluorescent N-terminal tag; or

iii) FAN1 overexpressing with EF1a HTLV promoter (no tag).

FAN1 overexpression in Huntington's Disease (HD) models are provide using an advanced adeno-associated virus (AAV) delivery system. The AAV plasmid overexpression construct includes several unique components:

Viral Dosages and Capsid: In some forms, the PhP.eB capsid is used. The PhP.eB capsid allows packaging the DNA and delivering it to cells, specifically in murine models due to its enhanced tropism for the central nervous system (CNS). For therapeutic applications in patients, however, AAV serotypes that are evolved for efficient usage and delivery to the CNS in primates/humans can be used (Mendoza, et al., J Neurophysiol. 117(5): 2004-2013.

Other useful capsids are known and reviewed in Campos, et al., Current Research in Neurobiology, Volume 4, 2023, 100086. In brief, to date, 13 distinct naturally-occurring or wildtype serotypes of AAVs, (AAV1-13) have been identified in humans and NHPs. Each of these serotypes differs in capsid structure and, therefore, tropism. The most commonly used serotypes in rodent research are AAV2, AAV5, AAV8, and AAV9, which transduce the CNS, although some transduce other organs as well (Aschauer et al., PLoS One, 8 (9) (2013), Article e76310). In NHPs, the most commonly used serotypes are AAV5 and AAV9 (Tremblay et al., Neuron, 108 (6) (2020), pp. 1075-1090.e6). AAV9 has been particularly widely studied because of its ability to cross the BBB and has been employed in several CNS-targeted gene therapies (Chan and Deverman, Crossing the Blood-Brain Barrier with AAVs: What's After SMA?. In: de Lange, E. C., Hammarlund-Udenaes, M., Thorne, R. G. (eds) Drug Delivery to the Brain. AAPS Advances in the Pharmaceutical Sciences Series, vol 33. Springer, Cham.); Chen et al., J. Contr. Release, 333 (2021), pp. 129-138). Gao et al. cloned and identified more than 100 novel rAAVs from human and NHP tissues (Gao et al., Curr. Gene Ther., 5 (3) (2005), pp. 285-297; Gao et al., Proc. Natl. Acad. Sci. USA, 99 (18) (2002), pp. 11854-11859). Among these, AAVrh8, AAVrh10 and AAVhu32 were found to cross the BBB with high efficiencies, similar to AAV9.

Alternative ITR Elements: Inverted terminal repeat (ITR) elements, crucial for the AAV's life cycle, including replication and packaging, are incorporated. This element is constant across different backbones.

Shortened 3′UTR Site: In these constructs, a shortened 3′ untranslated region (3′UTR) is utilized, which influences the regulation of gene expression. This site is constant across different constructs.

Mini Promoters: Mini promoters, including EF1a (for example, SEQ ID NO:16), EF1a-HTLV (for example, SEQ ID NO:17), hSYN (for example, SEQ ID NO:18), eSYN (for example, SEQ ID NO:19) and LAP2 (for example, SEQ ID NO:20), are in use. These DNA sequences initiate the transcription of a specific gene. The variety of promoters aims to regulate and optimize the FAN1 expression level. In some forms, the promoter is selected according to the desired anatomical location of the expressed FAN1 protein. Exemplary anatomical locations include muscle and neurons, as well as ubiquitous expression throughout all tissues/locations.

In some forms, the mini promoter is designed for ubiquitous expression of FAN1. An exemplary mini promoter for ubiquitous expression of FAN1 includes a core EF1a promoter. The core EF1a promoter drives ubiquitous promoter for broad expression in various cell types. An exemplary nucleic acid sequence for the core EF1a mini promoter is:

(SEQ ID NO: 16)
GGGCAGAGCGCACATCGCCCACAGTCCCCGAGAAGTTGGGGGGAGGGGT
CGGCAATTGAACGGGTGCCTAGAGAAGGTGGCGCGGGGTAAACTGGGAA
AGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTT
TGCCGCCAGAACACAG.

In some forms, the mini promoter designed for ubiquitous expression of FAN1 includes a core EF1a-HTLV promoter. EF1a-HTLV is a Human T-cell leukemia virus enhancer/promoter for ubiquitous expression. EF1a-HTLV is a composite promoter comprising the Elongation Factor-1α (EF-1α) core promoter and the R segment and part of the U5 sequence (R-U5′) of the Human T-Cell Leukemia Virus (HTLV) Type 1 Long Terminal Repeat. The EF-1α promoter exhibits a strong activity, higher than viral promoters and, on the contrary to the CMV promoter, yields persistent expression of the transgene in vivo. The R-U5′ has been coupled to the EF-1α core promoter to enhance stability of DNA and RNA. An exemplary nucleic acid sequence for the core EF1a-HTLV promoter is:

(SEQ ID NO: 17)
GGGCAGAGCGCACATCGCCCACAGTCCCCGAGAAGTTGGGGGGAGGGGT
CGGCAATTGAACGGGTGCCTAGAGAAGGTGGCGCGGGGTAAACTGGGAA
AGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTT
TGCCGCCAGAACACAGCTGAAGCTTCGAGGGGCTCGCATCTCTCCTTCA
CGCGCCCGCCGCCCTACCTGAGGCCGCCATCCACGCCGGTTGAGTCGCG
TTCTGCCGCCTCCCGCCTGTGGTGCCTCCTGAACTGCGTCCGCCGTCTA
GGTAAGTTTAAAGCTCAGGTCGAGACCGGGCCTTTGTCCGGCGCTCCCT
TGGAGCCTACCTAGACTCAGCCGGCTCTCCACGCTTTGCCTGACCCTGC
TTGCTCAACTCTACGTCTTTGTTTCGTTTTCTGTTCTGCGCCGTTACAG
ATCCAAGCTGTGACCGGCGCCTAC.

In some forms, the mini promoter is designed for neuronal expression of FAN1. An exemplary mini promoter for neuronal expression of FAN1 is LAP2, the 440 bp promoter sequence for high-level gene expression in neurons, in skeletal muscle and other peripheral tissues. An exemplary nucleic acid sequence for the LAP2 mini promoter is:

(SEQ ID NO: 20)
ATCCCCGGTCCGCGCTCCGCCCACCCATCACAGCAGCCGCGGACGCTGC
GCGCCGGAGCGGTCCATCTCGCCAGCCAGCCAACCAGCCGAGCCGCCCA
GCCGACCCGAGAGCCCCGAGAGCCAGACTCCCTCAGCCATAGAAGACAC
CGGGCGGGAGAGACGGACTGAAAAAATATATCTTTTTTTATTTTGTCTG
GGCCTGGAGACCCGCAGCAGGAGCGGAGGTGGGTGCGGGGCCGGGAGCC
GGAGCAGGACCGGGAACAGGAACAGGAACAGGAACAGGAACAGGAACAG
GAGTGGGGCCGGGAGCAGGAGCAGGAGCGGGAGCCGAAGTGGGGGCAGG
AGCGGCGGCGGCCGCAGCAGCAACAGGGTCGCCCCAGTCCGCGGCGAGG
AAGAGGGAGCTC.

In some forms, the Mini Promoter is a Human synapsin (hSYN) promoter for neuronal expression. An exemplary nucleic acid sequence for the hSYN1 mini promoter is:

(SEQ ID NO: 18)
AGTGCAAGTGGGTTTTAGGACCAGGATGAGGCGGGGTGGGGGTGCCTAC
CTGACGACCGACCCCGACCCACTGGACAAGCACCCAACCCCCATTCCCC
AAATTGCGCATCCCCTATCAGAGAGGGGGAGGGGAAACAGGATGCGGCG
AGGCGCGTGCGCACTGCCAGCTTCAGCACCGCGGACAGTGCCTTCGCCC
CCGCCTGGCGGCGCGCGCCACCGCCGCCTCAGCACTGAAGGCGCGCTGA
CGTCACTCGCCGGTCCCCCGCAAACTCCCCTTCCCGGCCACCTTGGTCG
CGTCCGCGCCGCCGCCGGCCCAGCCGGACCGCACCACGCGAGGCGCGAG
ATAGGGGGGCACGGGCGCGACCATCTGCGCTGCGGCGCCGGCGACTCAG
CGCTGCCTCAGTCTGCGGTGGGCAGCGGAGGAGTCGTGTCGTGCCTGAG
AGCGCAG.

In some forms, the Mini Promoter is an enhanced synapsin promoter for improved neuronal targeting (eSYN) promoter. An exemplary nucleic acid sequence for the eSYN mini promoter is:

(SEQ ID NO: 19)
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATGgAGTGCAAGTGGGTTTTAGGACCAGGATGAGGCGGGGTG
GGGGTGCCTACCTGACGACCGACCCCGACCCACTGGACAAGCACCCAAC
CCCCATTCCCCAAATTGCGCATCCCCTATCAGAGAGGGGGAGGGGAAAC
AGGATGCGGCGAGGCGCGTGCGCACTGCCAGCTTCAGCACCGCGGACAG
TGCCTTCGCCCCCGCCTGGCGGCGCGCGCCACCGCCGCCTCAGCACTGA
AGGCGCGCTGACGTCACTCGCCGGTCCCCCGCAAACTCCCCTTCCCGGC
CACCTTGGTCGCGTCCGCGCCGCCGCCGGCCCAGCCGGACCGCACCACG
CGAGGCGCGAGATAGGGGGGCACGGGCGCGACCATCTGCGCTGCGGCGC
CGGCGACTCAGCGCTGCCTCAGTCTGCGGTGGGCAGCGGAGGAGTCGTG
TCGTGCCTGAGAGCGCAG.

In some forms, the mini promoter is designed for muscle expression of FAN1. In some forms, the mini promoter designed for muscle expression of FAN1 is the MHCK7 promoter. The MHCK7 promoter is specifically optimized for muscle cells to drive FAN1 expression. An exemplary nucleic acid sequence for the MHCK7 mini promoter is:

(SEQ ID NO: 29)
CTAGACCCTTCAGATTAAAAATAACTGAGGTAAGGGCCTGGGTAGGGGAG
GTGGTGTGAGACGCTCCTGTCTCTCCTCTATCTGCCCATCGGCCCTTTGG
GGAGGAGGAATGTGCCCAAGGACTAAAAAAAGGCCATGGAGCCAGAGGGG
CGAGGGCAACAGACCTTTCATGGGCAAACCTTGGGGCCCTGCTGTCTAGC
ATGCCCCACTACGGGTCTAGGCTGCCCATGTAAGGAGGCAAGGCCTGGGG
ACACCCGAGATGCCTGGTTATAATTAACCCAGACATGTGGCTGCCCCCCC
CCCCCCAACACCTGCTGCCTCTAAAAATAACCCTGTCCCTGGTGGATCCC
CTGCATGCGAAGATCTTCGAACAAGGCTGTGGGGGACTGAGGGCAGGCTG
TAACAGGCTTGGGGGCCAGGGCTTATACGTGCCTGGGACTCCCAAAGTAT
TACTGTTCCATGTTCCCGGCGAAGGGCCAGCTGTCCCCCGCCAGCTAGAC
TCAGCACTTAGTTTAGGAACCAGTGAGCAAGTCAGCCCTTGGGGCAGCCC
ATACAAGGCCATGGGGCTGGGCAAGCTGCACGCCTGGGTCCGGGGTGGGC
ACGGTGCCCGGGCAACGAGCTGAAAGCTCATCTGCTCTCAGGGGCCCCTC
CCTGGGGACAGCCCCTCCTGGCTAGTCACACCCTGTAGGCTCCTCTATAT
AACCCAGGGGCACAGGGGCTGCCCTCATTCTACCACCACCTCCACAGCAC
AGACAGACACTCAGGAGCCAGCCAGCCTCGACCGGTCGATCTC.

In some forms, the Mini Promoter is an ultra-compact neuronal-specific promoter. An exemplary ultra compact neuronal-specific promoter is pCALM1. The pCALM1 promoter sequence originates from the human CALM1 gene and is only 120 bp in size, which allows for additional space for tagging of FAN1. An exemplary nucleic acid sequence for the pCALM mini promoter is:

(SEQ ID NO: 30)
CGCTCTGCGCGAGGCGGCTCCGCCGCGGCGGAGGGATACGGCGCACCATA
TATATATCGCGGGGCGCAGACTCGCGCTCCGGCAGTGGTGCTGGGAGTGT
CGTGGACGCCGTGCCGTTAC.

The pCLAM1 promoter is described in Chinese Patent No. CN112680443A.

NIR Fluorescent Protein Tag: In some forms, an NIR (near-infrared) fluorescent protein tag is attached to the N-terminus of FAN1 in the AAV construct, allowing for visual tracking of FAN1 expression in cells. In other instances for therapeutic application, this tag is not included in the transgene. In those instances, in some forms the start sequence of FAN1 is slightly modified so that a strong translation initiation Kozak sequence is provided, for example as provided in SEQ ID NO:22:

(SEQ ID NO: 22)
ATGGCAGAAGGGAAACCTCCTGACAAAAAAAGGCCTCGTAGAAGCTTATCAATCAGCAAGAAT
AAGAAAAAAGCATCTAATTCTATTATTTCGTGTTTTAACAATGCACCACCTGCTAAACTTGCC
TGCCCCGTTTGCAGTAAAATGGTGCCTAGATATGACTTAAACCGGCACCTTGATGAAATGTGT
GCTAACAATGACTTCGTTCAAGTGGATCCAGGGCAGGTTGGCTTAATAAATTCAAATGTGTCT
ATGGTAGATTTAACCAGTGTTACCTTAGAAGATGTAACACCTAAGAAGTCACCACCACCAAAG
ACAAATTTAACCCCTGGCCAAAGTGATTCAGCAAAAAGGGAAGTAAAGCAGAAGATCAGTCCC
TACTTTAAAAGTAATGATGTGGTGTGCAAAAATCAAGATGAGCTGAGAAATCGTAGTGTGAAA
GTCATTTGTTTGGGAAGCCTAGCATCTAAATTGTCCAGAAAATACGTAAAGGCTAAAAAATCA
ATAGATAAGGATGAAGAATTTGCCGGTTCTAGTCCACAGAGTTCCAAATGCACAGTTGTTAAG
AGCCTGATTGATAACTCTTCAGAAATTGAGGACGAGGATCAAATTTTGGAGAACAGTTCTCAA
AAAGAAAACGTGTTTAAATGTGATTCTCTAAAGGAAGAGTGCATTCCTGAACATATGGTAAGA
GGAAGTAAAATAATGGAAGCCGAAAGCCAAAAGGCTACCCGGGAATGTGAGAAATCAGCCCTC
ACCCCTGGATTCTCAGATAATGCGATCATGTTATTCTCACCAGATTTCACTCTTAGGAATACA
TTAAAGTCTACTTCAGAAGACAGTCTTGTAAAGCAAGAGTGTATCAAAGAAGTGGTTGAAAAA
CGTGAGGCATGTCATTGTGAAGAAGTAAAAATGACTGTTGCTTCAGAAGCTAAAATACAGCTG
TCAGATTCAGAGGCAAAATCTCATAGTTCTGCAGATGATGCTTCTGCATGGAGTAACATCCAA
GAGGCTCCTCTGCAGGATGACAGTTGCTTAAACAATGATATCCCTCACAGCATTCCTTTGGAG
CAGGGGTCAAGCTGCAATGGTCCTGGTCAAACAACCGGTCATCCTTACTACCTTCGGAGTTTC
CTTGTGGTGCTGAAAACCGTACTTGAGAATGAAGATGATATGTTGCTCTTTGATGAGCAGGAG
AAGGGAATTGTAACTAAATTTTATCAGTTATCAGCTACTGGTCAGAAGTTATATGTAAGGCTC
TTTCAACGTAAATTAAGCTGGATTAAGATGACCAAATTAGAGTATGAAGAGATTGCCTTAGAC
TTAACACCTGTGATTGAAGAATTGACGAATGCAGGCTTTCTACAGACAGAATCTGAGTTGCAA
GAACTCTCTGAAGTGCTTGAACTCCTTTCTGCTCCTGAACTAAAATCCCTAGCCAAGACCTTC
CACTTGGTGAATCCCAATGGACAGAAACAGCAGCTGGTGGACGCCTTTCTCAAATTGGCCAAA
CAGCGTTCAGTCTGCACTTGGGGCAAGAATAAGCCTGGAATTGGTGCAGTGATTTTAAAAAGA
GCCAAAGCCTTGGCTGGACAGTCAGTACGAATCTGTAAAGGCCCCAGGGCTGTGTTTTCCCGC
ATCTTGCTACTGTTTTCGTTGACCGACTCAATGGAAGATGAAGACGCCGCTTGTGGAGGTCAG
GGACAGCTTTCAACAGTCCTGTTGGTCAACCTCGGCCGAATGGAGTTTCCTAGTTACACCATC
AATCGGAAAACCCACATCTTCCAAGACAGAGATGATCTTATCAGATATGCAGCAGCCACGCAC
ATGCTGAGTGACATTTCTTCCGCAATGGCCAATGGGAACTGGGAAGAAGCTAAGGAGCTCGCT
CAGTGTGCAAAAAGGGATTGGAACAGACTGAAAAACCACCCTTCTCTGAGATGCCACGAAGAT
TTACCACTCTTCCTGCGGTGTTTCACTGTTGGGTGGATTTATACAAGGATTTTGTCTCGGTTT
GTGGAAATACTGCAGAGACTTCACATGTATGAGGAAGCCGTCAGAGAACTTGAAAGCCTTTTG
TCTCAGAGAATTTATTGTCCTGACAGCAGAGGCCGATGGTGGGATCGACTGGCCCTTAATTTA
CACCAGCACTTGAAGCGCCTGGAACCGACTATCAAGTGCATCACAGAGGGGCTGGCGGATCCG
GAAGTCAGAACGGGACACCGCCTTTCACTGTATCAGCGAGCCGTGCGCCTGCGAGAGTCTCCG
AGCTGTAAAAAGTTCAAGCACCTCTTCCAGCAGCTCCCAGAAATGGCTGTGCAAGATGTGAAA
CACGTGACCATCACAGGCAGGCTGTGCCCACAGCGTGGGATGTGCAAGTCTGTGTTTGTGATG
GAGGCCGGGGAGGCCGCTGACCCCACCACGGTCCTGTGCTCTGTGGAGGAGCTGGCACTGGCC
CATTACAGACGCAGCGGTTTTGACCAGGGGATTCATGGCGAAGGGTCCACCTTCAGCACCCTG
TATGGCCTCCTCCTGTGGGACATCATCTTCATGGATGGGATTCCGGATGTCTTCAGAAACGCC
TGTCAGGCATTCCCCCTGGACTTGTGCACAGACAGCTTCTTCACAAGCAGACGCCCAGCCCTT
GAGGCCAGGCTGCAGCTGATTCATGATGCCCCCGAGGAGAGCCTGCGGGCCTGGGTGGCAGCC
ACGTGGCATGAGCAGGAAGGCAGAGTGGCTTCCCTTGTCAGCTGGGATCGCTTCACGTCTCTT
CAGCAAGCTCAGGATCTTGTCTCCTGCCTGGGGGGCCCTGTGCTCAGTGGTGTGTGCAGGCAC
CTGGCTGCTGACTTTCGACACTGTCGAGGGGGCCTCCCCGACCTGGTGGTGTGGAACTCCCAG
AGCCGTCACTTTAAGCTGGTGGAAGTTAAAGGCCCCAATGATCGTCTTTCACATAAGCAGATG
ATCTGGCTGGCTGAACTGCAGAAGCTGGGGGCTGAAGTAGAAGTCTGCCATGTGGTTGCAGTT
GGAGCTAAGAGCCAAAGCCTTAGCTAA.

Large 3.1 Kb FAN1 Transgene: This construct is centered around the large ˜3.05 Kb FAN1 transgene, which is aimed to be overexpressed due to its potential therapeutic benefits in HD. Codon-Optimized FAN1 cDNA: In some instances, a codon-optimized FAN1 cDNA is used to enhance FAN1 protein production. This process involves modifying the DNA sequence of the FAN1 gene to improve its expression and translation. Multiple publicly available codon optimization tools (e.g., Genewiz, ThermoFisher, VectorBuilder, IDT, GenSmart, ExpOptimizer, etc.) and CUSTOM, a tissue-specific codon optimizer algorithm, are employed for this purpose (optimized here for expression in brain tissue see Hernandez-Alias, et al., Genome Biology, V. 24, No. 34 (2023)). A FAN1 codon-optimized promoter, SEQ ID NO:27. A consensus FAN1 codon-optimized promoter is provided in SEQ ID NO:28.

The AAV plasmid overexpression construct is a comprehensive system designed for effective delivery and overexpression of the FAN1 gene in HD models and for therapeutic application. It embodies multiple modifications to enhance gene expression efficiency and control.

v. N-Terminal Tags for Efficient Overexpression and Localization of FAN1

In some forms, the described constructs for overexpression and localization of FAN1 include one or more additional motifs, for example, appended to one or both terminals of the construct, to further enhance the expression of the FAN1 encoded by the construct, and/or to direct the expression of the construct to one or more anatomical locations in vivo.

In some forms, the constructs for expression of FAN1 include one or more motif including MyoSpreader (NoLS+NES), MIP/MIM binding flexible linker, NoLS (nucleolar localization) (and/or the miRFP670nano3 enhanced fluorescent protein for enhanced detection of expression).

a. Nucleolar Localization Sequence (NoLS) Tag

In some forms, the described constructs for overexpression and localization of FAN1 include a Nucleolar Localization Sequence (NoLS) Tag.

In some forms, attaching a nucleolar localization sequence (NoLS) tag to FAN1 can enhance its stability in the nucleus. The observed effects of similar localization sequences indicate that the stabilization and localization of other nucleases, such as the Cas9 nuclease, occur within the nucleolus. By localizing FAN1 to the nucleolus, the aim is to increase its stability and functional efficacy, thereby improving therapeutic outcomes for diseases characterized by repeat expansions.

Therefore, in some forms, the described constructs for over-expression of FAN1 include a NoLS motif. In some forms, a NoLS motif is appended to the N-terminus of the expression construct. An exemplary nucleic acid sequence for the NoLS motif is:

(SEQ ID NO: 33)
CGCAAAAAACGCAAAAAAAAA.

b. MIP/MIM Linker

In some forms, the described constructs for overexpression and localization of FAN1 include a Flexible linker containing MIP (SPYF) and/or MIM (LASKL) binding motifs.

The peptide binding motifs within FAN1 that interact with mismatch repair enzymes such as MLH1, MLH3, PMS2, and PMS1 are well characterized, specifically the MIP (SPYF) and MIM (LASKL) motifs. To enhance the binding efficiency of FAN1 to these mismatch repair enzymes, a flexible linker composed of a glycine-serine rich sequence, incorporating the MIP and MIM motifs at either end was engineered. This design aims to increase the interaction between FAN1 and mismatch repair enzymes, thereby amplifying the impact on repeat instability through an additive mechanism.

An exemplary nucleic acid sequence for the myospreader motif (NES+NoLS) is: TCTCCCTATTTTAGCGGAGGAGGTAGTGGCGGCGGCGGGTCCGGCGGAGGAGGCAGTGGTGGC GGGAGCCTCGCGTCCAAGCTGTCTCGA (SEQ ID NO:36).

c. MyoSpreader

In some forms, the described constructs for overexpression and localization of FAN1 include a Myospreader motif for FAN1 Propagation in Muscle Fibers.

In some forms, the described constructs include a MyoSpreader tag to enhance the propagation and distribution of FAN1 in muscle fibers. The MyoSpreader technology leverages a fusion protein approach that promotes widespread intracellular distribution of therapeutic proteins, ensuring comprehensive coverage within multinucleated muscle tissue. This approach significantly improves the efficiency of FAN1 delivery, a crucial factor for the treatment of muscle-specific pathologies like myotonic dystrophy.

An exemplary nucleic acid sequence for the myospreader motif (NES+NoLS) is:

(SEQ ID NO: 34)
GCTCCTCCGGCACAACCTCCCAGCCAACCTCAGCAGCATTATAGCGAAGG
AGAGTTGGAGGAAGATGAAGATTCTGATGACGCGCGCAAAAAACGCAAAA
AAAAA.

In some forms, the constructs further incorporate the MIP (SPYF) and/or MIM (LASKL) binding motifs. An exemplary nucleic acid sequence for the myospreader motif Full N-terminal tag with MIP/MIM linker is:

(SEQ ID NO: 35)
ATGGCTCCTCCGGCACAACCTCCCAGCCAACCTCAGCAGCATTATAGCGA
AGGAGAGTTGGAGGAAGATGAAGATTCTGATGACGCGCGCAAAAAACGCA
AAAAAAAATCTCCCTATTTTAGCGGAGGAGGTAGTGGCGGCGGCGGGTCC
GGCGGAGGAGGCAGTGGTGGCGGGAGCCTCGCGTCCAAGCTGTCTCGA.

iv. MYO-AAV Vector for Delivery to Muscle

In some forms, the constructs are incorporated within the myoAAV capsid for AAV delivery of fanI to muscle cells and application for treatment of myotonic dystrophy and other repeat expansion disorders.

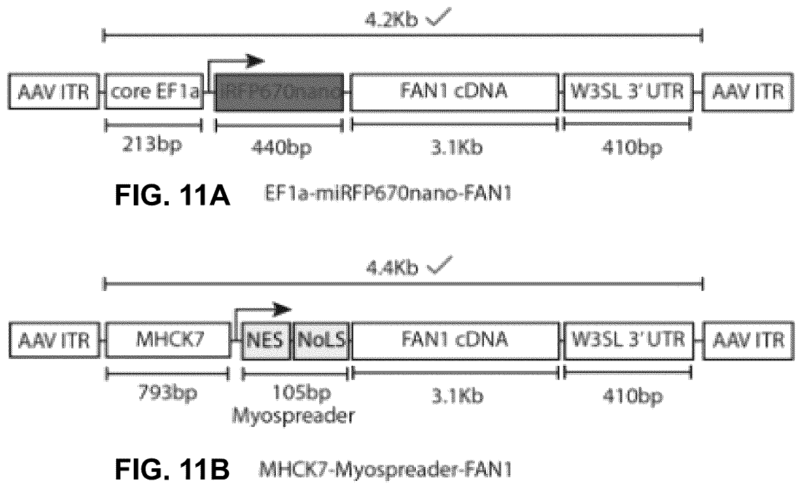

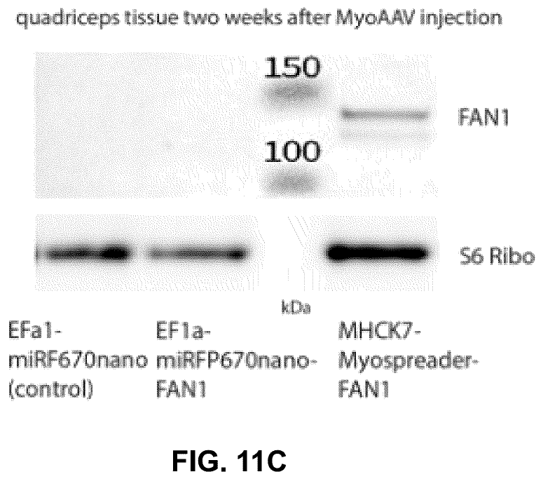

In some forms, to achieve effective gene delivery, the MyoAAV capsid, which is optimized for targeting muscle cells, is utilized. This capsid ensures efficient delivery and expression of the FAN1 gene in muscle tissue, a critical requirement for treating myotonic dystrophy and other repeat expansion disorders affecting muscle tissue. The use of MyoAAV capsids allows for targeted delivery, minimizing off-target effects and maximizing therapeutic impact. An exemplary Myo-AAV construct design is set forth in FIG. 11B.

The combination of the MyoSpreader tag, the NoLS tag for stabilization, and the MyoAAV capsid for targeted delivery represents a significant advancement in delivery and expression of FAN1 gene therapy for muscle-related disorders. Therefore, in some forms, the described compositions provide a therapeutic strategy for myotonic dystrophy and other similar conditions.

v. Exemplary AAV Vectors and Constructs Overexpressing FAN1

In some forms, the AAV overexpressing FAN1 is or includes
(i) the EF-1a core promoter,
(ii) a kozak sequence,
(iii) the iRFP670 nano tag,
(iv) the FAN1 sequence,
(v) the WSL 3′UTR; and
(vi) the AAV2 ITR.

An exemplary vector includes the nucleic acid sequence:

(SEQ ID NO: 21)
gggcagagcgcacatcgcccacagtccccgagaagttggggggaggggtcggcaattgaaccg
gtgcctagagaaggtggcgcggggtaaactgggaaagtgatgtcgtgtactggctccgccttt
ttcccgagggtgggggagaaccgtatataagtgcagtagtcgccgtgaacgttctttttcgca
acgggtttgccgccagaacacaggatctgccgccaccATGGCAAACCTGGACAAGATGCTGAA
TACCACAGTAACAGAGGTGCGGCAGTTCCTGCAGGTGGACAGAGTGTGCGTGTTCCAGTTTGA
GGAGGATTATAGCGGAGTGGTGGTGGTGGAGGCCGTGGACGATAGGTGGATCTCCATCCTGAA
GACCCAGGTGCGGGATAGATACTTCATGGAGACAAGGGGCGAGGAGTATTCTCACGGCCGCTA
CCAGGCCATCGCCGACATCTACACCGCAAACCTGACAGAGTGCTACAGGGATCTGCTGACACA
GTTTCAGGTGAGAGCAATCCTGGCCGTGCCCATCCTGCAGGGCAAGAAGCTGTGGGGCCTGTT
GGTGGCACACCAGCTGGCGGCCCCTAGACAGTGGCAGACCTGGGAGATCGACTTTCTGAAGCA
GCAGGCCGTGGTGGTGGGCATCGCCATCCAGCAGAGCGGAGGTGGTGGATCAGGAGGAGGTGG
TTCTGGAGGTGGTGGATCAGGAGGAAGATCTCGAGCTATGTCAGAAGGGAAACCTCCTGACAA
AAAAAGGCCTCGTAGAAGCTTATCAATCAGCAAGAATAAGAAAAAAGCATCTAATTCTATTAT
TTCGTGTTTTAACAATGCACCACCTGCTAAACTTGCCTGCCCCGTTTGCAGTAAAATGGTGCC
TAGATATGACTTAAACCGGCACCTTGATGAAATGTGTGCTAACAATGACTTCGTTCAAGTGGA
TCCAGGGCAGGTTGGCTTAATAAATTCAAATGTGTCTATGGTAGATTTAACCAGTGTTACCTT
AGAAGATGTAACACCTAAGAAGTCACCACCACCAAAGACAAATTTAACCCCTGGCCAAAGTGA
TTCAGCAAAAAGGGAAGTAAAGCAGAAGATCAGTCCCTACTTTAAAAGTAATGATGTGGTGTG
CAAAAATCAAGATGAGCTGAGAAATCGTAGTGTGAAAGTCATTTGTTTGGGAAGCCTAGCATC
TAAATTGTCCAGAAAATACGTAAAGGCTAAAAAATCAATAGATAAGGATGAAGAATTTGCCGG
TTCTAGTCCACAGAGTTCCAAATgCACAGTTGTTAAGAGCCTGATTGATAACTCTTCAGAAAT
TGAGGACGAGGATCAAATTTTGGAGAACAGTTCTCAAAAAGAAAACGTGTTTAAATGTGATTC
TCTAAAGGAAGAGTGCATTCCTGAACATATGGTAAGAGqAAGTAAAATAATGGAAGCCGAAAG
CCAAAAGGCTACCCGGGAATGTGAGAAATCAGCCCTCACCCCTGGATTCTCAGATAATGCGAT
CATGTTATTCTCACCAGATTTCACTCTTAGGAATACATTAAAGTCTACTTCAGAAGACAGTCT
TGTAAAGCAAGAGTGTATCAAAGAAGTGGTTGAAAAACGTGAGGCATGTCATTGTGAAGAAGT
AAAAATGACTGTTGCTTCAGAAGCTAAAATACAGCTGTCAGATTCAGAGGCAAAATCTCATAG
TTCTGCAGATGATGCTTCTGCATGGAGTAACATCCAAGAGGCTCCTCTGCAGGATGACAGTTG
CTTAAACAATGATATCCCTCACAGCATTCCTTTGGAGCAGGGGTCAAGCTGCAATGGTCCTGG
TCAAACAACCGGTCATCCTTACTACCTTCGGAGTTTCCTTGTGGTGCTGAAAACCGTACTTGA
GAATGAAGATGATATGTTGCTCTTTGATGAGCAGGAGAAGGGAATTGTAACTAAATTTTATCA
GTTATCAGCTACTGGTCAGAAGTTATATGTAAGGCTCTTTCAACGTAAATTAAGCTGGATTAA
GATGACCAAATTAGAGTATGAAGAGATTGCCTTAGACTTAACACCTGTGATTGAAGAATTGAC
GAATGCAGGCTTTCTACAGACAGAATCTGAGTTGCAAGAACTCTCTGAAGTGCTTGAACTCCT
TTCTGCTCCTGAACTAAAATCCCTAGCCAAGACCTTCCACTTGGTGAATCCCAATGGACAGAA
ACAGCAGCTGGTGGACGCCTTTCTCAAATTGGCCAAACAGCGTTCAGTCTGCACTTGGGGCAA
GAATAAGCCTGGAATTGGTGCAGTGATTTTAAAAAGAGCCAAAGCCTTGGCTGGACAGTCAGT
ACGAATCTGTAAAGGCCCCAGGGCTGTGTTTTCCCGCATCTTGCTACTGTTTTCGTTGACCGA
CTCAATGGAAGATGAAGACGCCGCTTGTGGAGGTCAGGGACAGCTTTCAACAGTCCTGTTGGT
CAACCTCGGCCGAATGGAGTTTCCTAGTTACACCATCAATCGGAAAACCCACATCTTCCAAGA
CAGAGATGATCTTATCAGATATGCAGCAGCCACGCACATGCTGAGTGACATTTCTTCCGCAAT
GGCCAATGGGAACTGGGAAGAAGCTAAGGAGCTCGCTCAGTGTGCAAAAAGGGATTGGAACAG
ACTGAAAAACCACCCTTCTCTGAGATGCCACGAAGATTTACCACTCTTCCTGCGGTGTTTCAC
TGTTGGGTGGATTTATACAAGGATTTTGTCTCGGTTTGTGGAAATACTGCAGAGACTTCACAT
GTATGAGGAAGCCGTCAGAGAACTTGAAAGCCTTTTGTCTCAGAGAATTTATTGTCCTGACAG
CAGAGGCCGATGGTGGGATCGACTGGCCCTTAATTTACACCAGCACTTGAAGCGCCTGGAACC
GACTATCAAGTGCATCACAGAGGGGCTGGCGGATCCGGAAGTCAGAACGGGACACCGCCTTTC
ACTGTATCAGCGAGCCGTGCGCCTGCGAGAGTCTCCGAGCTGTAAAAAGTTCAAGCACCTCTT
CCAGCAGCTCCCAGAAATGGCTGTGCAAGATGTGAAACACGTGACCATCACAGGCAGGCTGTG
CCCACAGCGTGGGATGTGCAAGTCTGTGTTTGTGATGGAGGCCGGGGAGGCCGCTGACCCCAC
CACGGTCCTGTGCTCTGTGGAGGAGCTGGCACTGGCCCATTACAGACGCAGCGGTTTTGACCA
GGGGATTCATGGCGAAGGGTCCACCTTCAGCACCCTGTATGGCCTCCTCCTGTGGGACATCAT
CTTCATGGATGGGATTCCGGATGTCTTCAGAAACGCCTGTCAGGCATTCCCCCTGGACTTGTG
CACAGACAGCTTCTTCACAAGCAGACGCCCAGCCCTTGAGGCCAGGCTGCAGCTGATTCATGA
TGCCCCCGAGGAGAGCCTGCGGGCCTGGGTGGCAGCCACGTGGCATGAGCAGGAAGGCAGAGT
GGCTTCCCTTGTCAGCTGGGATCGCTTCACGTCTCTTCAGCAAGCTCAGGATCTTGTCTCCTG
CCTGGGGGGCCCTGTGCTCAGTGGTGTGTGCAGGCACCTGGCTGCTGACTTTCGACACTGTCG
AGGGGGCCTCCCCGACCTGGTGGTGTGGAACTCCCAGAGCCGTCACTTTAAGCTGGTGGAAGT
TAAAGGCCCCAATGATCGTCTTTCACATAAGCAGATGATCTGGCTGGCTGAACTGCAGAAGCT
GGGGGCTGAAGTAGAAGTCTGCCAtGTGGTTGCAGTTGGAGCTAAGAGCCAAAGCCTTAGCTA
Agaattccgctcgagataatcaacctctggattacaaaatttgtgaaagattgactggtattc
ttaactatgttgctccttttacgctatgtggatacgctgctttaatgcctttgtatcatgcta
ttgcttcccgtatggctttcattttctcctccttgtataaatcctggttagttcttgccacgg
cggaactcatcgccgcctgccttgcccgctgctggacaggggctcggctgttgggcactgaca
attccgtggtgtttatttgtgaaatttgtgatgctattgctttatttgtaaccatctagcttt
atttgtgaaatttgtgatgctattgctttatttgtaaccattataagctgcaataaacaagtt
aacaacaacaattgcattcattttatgtttcaggttcagggggagatgtgggaggttttttaa
agcggccgcAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTGAGCG
AGCGAGCGCGCAGCTGCCTGCAGG.

### B. Compositions for Reducing Expression of Alternatively Spliced Nonfunctional FAN1

i. FAN1 Expression-TG Repeat-Targeting ASO (TGASO)

ASOs can be designed to block protein translation through Watson-Crick base-pairing with the target mRNA and can be modified to improve stability. The ASOs, however, inhibit protein production through a variety of mechanisms, such as sterically blocking ribosome attachment or eliciting RNase-H activation. They can also promote exon skipping (a form of RNA splicing which leaves out faulty exons), which allows for the deletion of faulty sequences within proteins

In some forms, the ASO specifically targets the TG repeats of FAN1 mRNA has the nucleotide sequence of any one of SEQ ID NOs:10-12.
ACAAGGTCACACACACA (SEQ ID NO:10, “ASO3”, where T is a 5-methyl Uridine nucleotide and C is a 5-methyl Cytidine nucleotide),
AGGTCACACACACACACA (SEQ ID NO:11, “ASO5”, where T is a 5-methyl Uridine nucleotide and C is a 5-methyl Cytidine nucleotide), and

GTCACACACACACACACA (SEQ ID NO: 12, “ASO6”, where T
is a 5-methyl Uridine nucleotide and C is a 5-
methyl Cytidine nucleotide),

where T are 5-methyl Uridine bases and C are 5-methyl Cytidines.

In some embodiments, one of more TGASOs that can decrease the expression of FAN1 has the nucleotide sequence of any one of SEQ ID NOs:10-12.

In further embodiments, one or more non-targeting control ASOs are used. An exemplary non-targeting control ASO targets beta-thalassemia mutation. In one embodiment, the non-targeting control ASO has the nucleotide sequence of

CCTCTTACCTCAGTTACAATTTATA (SEQ ID NO: 13, “nt” or
“control”, where T is a 5-methyl Uridine nucleo-
tide and C is a 5-methyl Cytidine nucleotide.

ii. Editing DNA/mRNA FAN1 DNA with Unwanted TG Repeats Using CRISPR System

In some embodiments, one or more segments of the detained intron within intron 10 of FAN1 mRNA is edited, for example using CRISPR/Cas technology. The CRISPR/Cas system is naturally composed of one or several CRISPR-associated (Cas) proteins and a genomic region termed Clustered Regularly Interspaced Short Palindromic Repeats (CRISPR) which memorizes previous pathogen attacks. For engineering purposes, the CRISPR-Cas system has been adapted such that only a single guide RNA molecule (sgRNA) and, generally, only one Cas protein are needed to bind and, in some cases, cleave a nucleic acid target. Notably, the system only binds and activates its catalytic function if the target contains a sequence that is complementary to that defined in the spacer region of the guide RNA molecule.

The main components of CRISPR-Cas9 system are RNA-guided Cas9 endonuclease and an sgRNA. The Cas9 protein possesses two nuclease domains, named HNH and RuvC, and each cleaves one strand of the target double-stranded DNA. An sgRNA is a simplified combination of crRNA and tracrRNA. The Cas9 nuclease and sgRNA form a Cas9 ribonucleoprotein (RNP), which can bind and cleave the specific DNA target. Furthermore, a protospacer adjacent motif (PAM) sequence is required for Cas9 protein's binding to the target DNA. However, multiple studies have harnessed Cas9 for RNA targeting under specific circumstances. For example, the S. pyogenes Cas9 (SpyCas9) can be supplied with a short DNA oligo containing the PAM sequence (a PAMmer) to induce single-stranded RNA (ssRNA) binding and cutting. More recently, it was demonstrated that SpyCas9 could be used to target repetitive RNAs and repress translation in certain mRNAs in the absence of a PAMmer. A different Cas9 homolog from Francisella novicida (FnoCas9) has been implicated in degradation of a specific mRNA but through a mechanism independent of RNA-based cleavage. Thus, some Cas9 homologs can target single-stranded substrates (reviewed in Strutt, et al., eLife. 2018; 7: e32724).

The recent discovery of the class II type VI CRISPR/Cas13 system further expands the existing CRISPR technology (Cox DBT, et al., Science. 358:1019-27 (2017)).

Like Cas9, Cas13 uses a guide RNA (CRISPR-RNA, aka crRNA) to identify its substrate, which is RNA rather than DNA. Cas13 enzymes have two distinct catalytic activities: (i) an RNAse activity that is mediated by two higher eukaryotic and prokaryotic nucleotide (HEPN)-binding domains and (ii) a gRNA maturation activity, possibly a combination of activities located in the HEPN2 and Helical-1 domains. There are currently four subtypes identified in the Cas13 family, including Cas13a (aka C2c2), Cas13b, Cas13c, and Cas13d. All Cas13 family members are smaller than Cas9, with Cas13d being the smallest protein. In some forms, a dCasRx (a member of the Cas13d family, also known as RfxCas13d) system can be used to steric hinder splicing machinery similarly to the SSOs by targeting dCasRx to the 5′ end of the detained intron by using same target sequence for a gRNA as the ASO17/SSO target sequence. Similarly, these gRNAs could be used to guide CasFx, artificial splicing factors that tether dCasRx with RBFOX1 or RBM38 fusions, to the target sequences herein identified in FAN1 intron10 to enhance the modulation of adjacent splicing events. (Du, et al., Nature Communications. 2020; REF: https://www.nature.com/articles/s41467-020-16806-4)

Cas7-11 is a recently characterized type III-E CRISPR-Cas effector that catalyzes pre-crRNA processing and crRNA-guided target RNA cleavage. Cas7-11 associates with a crRNA and cleaves a complementary ssRNA target at two defined positions 6-nt apart, using the conserved catalytic residues in the second and third Cas7 domains. Cas7-11 processes its own pre-crRNAs to produce mature crRNAs. Cas7-11 specifically cleaves a target RNA complementary to the crRNA guide and thus exhibits less cell toxicity, can be used as a programmable RNA-targeting tool without collateral activity and cell toxicity and has been applied to RNA knockdown and editing in mammalian cells. (van Beljouw et al., Science. 2021; 373(6561):1349-1353; Özcan et al. The Cas enzyme and guide RNAs can be delivered as RNP complexes or can be expressed using a vector such as a plasmid or virus.

Kato, et al., (Cell, 185 (13): 2324-2337 (2022) rationally engineered a compact Cas7-11 variant (Cas7-11S) for single-vector AAV packaging for transcript knockdown in human cells, enabling in vivo Cas7-11 applications.

In some embodiments, an RNA-targeting nuclease such as Cas9 (designed to target RNA), Cas13, Cas7-11 or Cas7-11S is used to specifically target one or more regions of the detained intron within intron 10 of FAN1 mRNA. In preferred embodiments, an RNA-targeting nuclease effectively modify one or more regions of the detained intron within intron 10 (SEQ ID NO:2) of FAN1 mRNA to become a more thermodynamically stable stem loop (TG-stem) to improve splicing at this junction in FAN1. In other embodiments, the Cas system targets DNA to edit/shorten/delete the TG repeats (TG-del).

### C. Chemical Modifications of ASOs

Natural, unmodified DNA and RNA oligonucleotides are generally unfavorable as therapeutics because they are vulnerable to nuclease degradation in serum and cells and thereby are unstable in vivo. To be effective, all ASOs require chemical modification to resist nuclease degradation. Chemical modifications have improved oligonucleotide binding affinity, stability and pharmacodynamic properties. Modifications that improve these qualities involve changes to the phosphate backbone and/or sugar component of the oligonucleotide.

Nucleotides of an SSASO are chemically modified so that the RNA-cleaving enzyme RNase H is not recruited to degrade the pre-mRNA-SSO complex. The RNAse H-resistant features of SSASOs are critical because the goal of SSOs is to alter splicing and not to cause the degradation of the bound pre-mRNA. The phosphorothiate (PS) backbone modification was the first analog to be used in clinical applications and has been incorporated into many SSASO designs that are currently being developed as potential therapeutics. SSASOs with a PS backbone modification have modestly reduced binding affinities but have improved stability in vivo, with greater nuclease resistance. PS SSASOs also bind to proteins in plasma, which reduces renal clearance and improves retention, allowing for broad biodistribution, but also increasing the risk of toxicity. Accordingly, in some embodiments, the nucleotides of the SSASOs or TGASO all have phosphorothiate backbone modification.

Oligonucleotides with PS backbone modifications are not resistant to RNAse H and thus, to create a steric blocking SSASO for splice-switching applications, additional modifications to the molecule are required. ASOs that are fully modified at the 2′ sugar position confer RNAse H-resistance and are commonly used as SSASOs. The most widely used alterations at the 2′ position are 2′-O-methyl (2′-OMe) and 2′-O-methoxyethyl (2′-MOE). Accordingly, in some embodiments, the nucleotides of the SSASOs all 2′-O-methoxyethylribose (MOE) bases.

Locked nucleic acid (LNA) chemistry, is another modification of the sugar, which involves bridging of the furanose ring. A major benefit of LNA modified SSOs is the elevated binding affinity, which is an important consideration as high binding affinity can allow for the use of shorter SSASO sequences. A shorter sequence can reduce the likelihood of binding to an incorrect site as a result of partial sequence complementarity to another sequence and thus lower the risk of unwanted off-target effects. Thus, in some embodiments, the nucleotides of the SSSOs or TGASOs have one or more of these chemical modifications such as 2′-OMe/PS, 2′-MOE/PS; LNA/PS.

Phosphorodiamidate morpholinos (PMOs) are another type of modified oligonucleotide that has been used extensively to modify splicing. PMOs have a morpholine ring in place of the furanose ring found in natural nucleic acids and a neutral phosphorodiamidate backbone in place of the negatively charged phosphodiester backbone. In some embodiments, the chemical modifications of the nucleotides of the splice switching ASOs involve PMOs.

Preferably, the nucleotides of the SSASO are all 2′-O-methoxyethylribose (MOE) bases, all phosphorothioate (SOX) backbone linkages.

One of ordinary skill in the art can readily design SSASO's that specifically target the 5′ end of the detained intron within intron 10 of FAN1 mRNA as disclosed herein, using methods known in the art. Exemplary methods for designing SSASO's are disclosed in WO 2023/009396, which does not recognize the specific targets disclosed herein (i.e., the detained intron within intron 10 of FAN1 mRNA) or its significance within the context of trinucleotide repeat expansion disorders.

### C. Delivery Vehicles

In some embodiments, compositions of one or more splice switching ASOs or CRISPR/Cas component include one or more particles for delivery into the body.

A preferred AAV for delivery of FAN1 overexpression is the AAV-delivery vehicle disclosed herein In one embodiment, the AAVs are engineered for efficient noninvasive gene delivery (retro-orbital) to the central nervous system using a capsid selection method similar to those described in Chan et al., (Nat Neurosci. 2017 August; 20(8):1172-1179).

Appropriate delivery vehicles for the compounds are known in the art and can be selected to suit the particular compositions. For example, in some embodiments, the composition is incorporated into or encapsulated by, or bound to, a nanoparticle, microparticle, microsphere, micelle, natural or synthetic lipoprotein particle, liposomal nanoparticle, or dendrimeric particle. In other embodiments, the composition is incorporated into or encapsulated by or bound to one or more cationic polymers.

### 1. Lipidic Particles

In some embodiments, the particle is a lipid particle, liposome, or micelle, or includes a lipid core. Lipid particles and lipid nanoparticles are known in the art. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

Representative neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol.

Representative cationic lipids include, but are not limited to, N-[1-(2,3-dioleoyloxy)propyl]-N,N,N-trimethyl ammonium salts, referred to as TAP lipids, for example, methylsulfate salt. Representative TAP lipids include, but are not limited to, DOTAP (dioleoyl-), DMTAP (dimyristoyl-), DPTAP (dipalmitoyl-), and DSTAP (distearoyl-). Representative cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

a. Micelles

In some embodiments, the particle or particle core is a lipid micelle. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

b. Liposomes

In some embodiments, the particle or particle core is a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate targeted agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Representative alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

c. Solid Lipid Particles

In some embodiments, the particle is a solid lipid particle, or includes a solid lipid core. Solid lipid particles present an alternative to the colloidal micelles and liposomes. Solid lipid particles are typically submicron in size, i.e., from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Representative solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Representative solid lipids can include cetyl palmitate or beeswax. Cyclodextrin can also be used.

### 3. Nanoparticle Delivery Vehicles

Any of the disclosed compositions including, SSASO or gene editing molecules, donor oligonucleotides, etc., can be delivered to the target cells using a nanoparticle delivery vehicle.

Nanoparticles generally refers to particles in the range of between 500 nm to less than 0.5 nm, preferably having a diameter that is between 50 and 500 nm, more preferably having a diameter that is between 50 and 300 nm. Cellular internalization of polymeric particles is highly dependent upon their size, with nanoparticulate polymeric particles being internalized by cells with much higher efficiency than microparticulate polymeric particles. For example, Desai, et al. have demonstrated that about 2.5 times more nanoparticles that are 100 nm in diameter are taken up by cultured Caco-2 cells as compared to microparticles having a diameter on 1 μM (Desai, et al., Pharm. Res., 14:1568-73 (1997)). Nanoparticles also have a greater ability to diffuse deeper into tissues in vivo.

a. Polymer

The polymer that forms the core of the nanoparticle may be any biodegradable or non-biodegradable synthetic or natural polymer. In a preferred embodiment, the polymer is a biodegradable polymer. Nanoparticles are ideal materials for the fabrication of gene editing delivery vehicles: 1) control over the size range of fabrication, down to 100 nm or less, an important feature for passing through biological barriers; 2) reproducible biodegradability without the addition of enzymes or cofactors; 3) capability for sustained release of encapsulated, protected nucleic acids over a period in the range of days to months by varying factors such as the monomer ratios or polymer size, for example, the ratio of lactide to glycolide monomer units in poly(lactide-co-glycolide) (PLGA); 4) well-understood fabrication methodologies that offer flexibility over the range of parameters that can be used for fabrication, including choices of the polymer material, solvent, stabilizer, and scale of production; and 5) control over surface properties facilitating the introduction of modular functionalities into the surface.

Examples of preferred biodegradable polymers include synthetic polymers that degrade by hydrolysis such as poly(hydroxy acids), such as polymers and copolymers of lactic acid and glycolic acid, other degradable polyesters, polyanhydrides, poly(ortho)esters, polyesters, polyurethanes, poly(butic acid), poly(valeric acid), poly(caprolactone), poly(hydroxyalkanoates), poly(lactide-co-caprolactone), and poly(amine-co-ester) polymers, such as those described in Zhou, et al., Nature Materials, 11:82-90 (2012) and WO 2013/082529, U.S. Published Application No. 2014/0342003, and PCT/US2015/061375.

Preferred natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate). These materials may be used alone, as physical mixtures (blends), or as co-polymers.

The polymer may be a bioadhesive polymer that is hydrophilic or hydrophobic. Hydrophilic polymers include CARBOPOL™ (a high molecular weight, crosslinked, acrylic acid-based polymers manufactured by NOVEON™), polycarbophil, cellulose esters, and dextran.

Release rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and 5100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, MO; Polysciences, Warrenton, PA; Aldrich, Milwaukee, WI; Fluka, Ronkonkoma, NY; and BioRad, Richmond, CA, or can be synthesized from monomers obtained from these or other suppliers using standard techniques.

In a preferred embodiment, the nanoparticles are formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)). These polymers have been used to encapsulate siRNA (Yuan, et al., Jour. Nanosocience and Nanotechnology, 6:2821-8 (2006); Braden, et al., Jour. Biomed. Nanotechnology, 3:148-59 (2007); Khan, et al., Jour. Drug Target, 12:393-404 (2004); Woodrow, et al., Nature Materials, 8:526-533 (2009)). Murata, et al., J. Control. Release, 126(3):246-54 (2008) showed inhibition of tumor growth after intratumoral injection of PLGA microspheres encapsulating siRNA targeted against vascular endothelial growth factor (VEGF). However, these microspheres were too large to be endocytosed (35-45 μm) (Conner and Schmid, Nature, 422(6927):37-44 (2003)) and required release of the anti-VEGF siRNA extracellularly as a polyplex with either polyarginine or PEI before they could be internalized by the cell. These microparticles may have limited applications because of the toxicity of the polycations and the size of the particles. Nanoparticles (100-300 nm) of PLGA can penetrate deep into tissue and are easily internalized by many cells (Conner and Schmid, Nature, 422(6927):37-44 (2003)).

The nanoparticles can be designed to release encapsulated nucleic acids over a period of days to weeks. Factors that affect the duration of release include pH of the surrounding medium (higher rate of release at pH 5 and below due to acid catalyzed hydrolysis of PLGA) and polymer composition. Aliphatic polyesters differ in hydrophobicity, affecting degradation rate. Specifically, the hydrophobic poly (lactic acid) (PLA), more hydrophilic poly (glycolic acid) PGA and their copolymers, poly (lactide-co-glycolide) (PLGA) have various release rates. The degradation rate of these polymers, and often the corresponding drug release rate, can vary from days (PGA) to months (PLA) and is easily manipulated by varying the ratio of PLA to PGA.

Exemplary nanoparticles are described in U.S. Pat. Nos. 4,883,666, 5,114,719, 5,601,835, 7,534,448, 7,534,449, 7,550,154, and 8,889,117, and U.S. Published Application Nos. 2009/0269397, 2009/0239789, 2010/0151436, 2011/0008451, 2011/0268810, 2014/0342003, 2015/0118311, 2015/0125384, 2015/0073041, Hubbell, et al., Science, 337:303-305 (2012), Cheng, et al., Biomaterials, 32:6194-6203 (2011), Rodriguez, et al., Science, 339:971-975 (2013), Hrkach, et al., Sci Transl Med., 4:128ra139 (2012), McNeer, et al., Mol Ther., 19:172-180 (2011), McNeer, et al., Gene Ther., 20:658-659 (2013), Babar, et al., Proc Natl Acad Sci USA, 109:E1695-E1704 (2012), Fields, et al., J Control Release 164:41-48 (2012), and Fields, et al., Advanced Healthcare Materials, 361-366 (2015).

b. Polycations

In some forms, the nucleic acids are complexed to polycations to increase the encapsulation efficiency of the nucleic acids into the nanoparticles. The term “polycation” refers to a compound having a positive charge, preferably at least 2 positive charges, at a selected pH, preferably physiological pH. Polycationic moieties have between about 2 to about 15 positive charges, preferably between about 2 to about 12 positive charges, and more preferably between about 2 to about 8 positive charges at selected pH values.

Many polycations are known in the art. Suitable constituents of polycations include basic amino acids and their derivatives such as arginine, asparagine, glutamine, lysine and histidine; cationic dendrimers; and amino polysaccharides. Suitable polycations can be linear, such as linear tetralysine, branched or dendrimeric in structure.

Exemplary polycations include, but are not limited to, synthetic polycations based on acrylamide and 2-acrylamido-2-methylpropanetrimethylamine, poly(N-ethyl-4-vinylpyridine) or similar quartemized polypyridine, diethylaminoethyl polymers and dextran conjugates, polymyxin B sulfate, lipopolyamines, poly(allylamines) such as the strong polycation poly(dimethyldiallylammonium chloride), polyethyleneimine, polybrene, and polypeptides such as protamine, the histone polypeptides, polylysine, polyarginine and polyornithine.

In one embodiment, the polycation is a polyamine. Polyamines are compounds having two or more primary amine groups. In a preferred embodiment, the polyamine is a naturally occurring polyamine that is produced in prokaryotic or eukaryotic cells. Naturally occurring polyamines represent compounds with cations that are found at regularly-spaced intervals and are therefore particularly suitable for complexing with nucleic acids. Polyamines play a major role in very basic genetic processes such as DNA synthesis and gene expression. Polyamines are integral to cell migration, proliferation and differentiation in plants and animals. The metabolic levels of polyamines and amino acid precursors are critical and hence biosynthesis and degradation are tightly regulated. Suitable naturally occurring polyamines include, but are not limited to, spermine, spermidine, cadaverine and putrescine. In a preferred embodiment, the polyamine is spermidine.

In another embodiment, the polycation is a cyclic polyamine. Cyclic polyamines are known in the art and are described, for example, in U.S. Pat. No. 5,698,546, WO 1993/012096 and WO 2002/010142. Exemplary cyclic polyamines include, but are not limited to, cyclen.

Spermine and spermidine are derivatives of putrescine (1,4-diaminobutane) which is produced from L-ornithine by action of ODC (ornithine decarboxylase). L-ornithine is the product of L-arginine degradation by arginase. Spermidine is a triamine structure that is produced by spermidine synthase (SpdS) which catalyzes monoalkylation of putrescine (1,4-diaminobutane) with decarboxylated S-adenosylmethionine (dcAdoMet) 3-aminopropyl donor. The formal alkylation of both amino groups of putrescine with the 3-aminopropyl donor yields the symmetrical tetraamine spermine. The biosynthesis of spermine proceeds to spermidine by the effect of spermine synthase (SpmS) in the presence of dcAdoMet. The 3-aminopropyl donor (dcAdoMet) is derived from S-adenosylmethionine by sequential transformation of L-methionine by methionine adenosyltransferase followed by decarboxylation by AdoMetDC (S-adenosylmethionine decarboxylase). Hence, putrescine, spermidine and spermine are metabolites derived from the amino acids L-arginine (L-ornithine, putrescine) and L-methionine (dcAdoMet, aminopropyl donor).

In some embodiments, the particles themselves are a polycation (e.g., a blend of PLGA and poly(beta amino ester).

### D. Pharmaceutically Acceptable Carriers

In some embodiments, the compositions include one or more pharmaceutically acceptable carriers, or excipients, or preservatives. Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable carriers include, but are not limited to, buffers, diluents, preservatives, binders, stabilizers, a mixture, or polymer of sugars (lactose, sucrose, dextrose, etc.), salts, and combinations thereof.

The compositions may be administered in combination with one or more physiologically or pharmaceutically acceptable carriers, thickening agents, co-solvents, adhesives, antioxidants, buffers, viscosity, and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

In general, pharmaceutical compositions are provided including effective amounts of the composition, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as antioxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

In some embodiments, the pharmaceutical composition is a saline solution, preferably a buffered saline solution phosphate buffered saline or sterile saline, or tissue culture medium.

### 1. Formulations

The disclosed compositions can be formulated in a pharmaceutical composition. Pharmaceutical compositions including antigens, adjuvants, and the combination thereof are provided. Pharmaceutical compositions can be for administration by parenteral (intramuscular, intraperitoneal, intravenous (IV) or subcutaneous injection), enteral, transdermal (either passively or using iontophoresis or electroporation), or using bioerodible inserts and can be formulated in dosage forms appropriate for each route of administration.

In some embodiments, the compositions are administered systemically, for example, by intravenous or intraperitoneal administration, in an amount effective for delivery of the compositions to targeted cells.

Most typically, the compositions are administered by intramuscular, intradermal, subcutaneous injection or infusions, or intravenous injection or infusion, or by intranasal delivery.

Compositions and pharmaceutical formulations thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria-retaining filter, by incorporating sterilizing agents into the compositions, by irradiating the compositions, or by heating the compositions.

### E. Minigene Reporter System

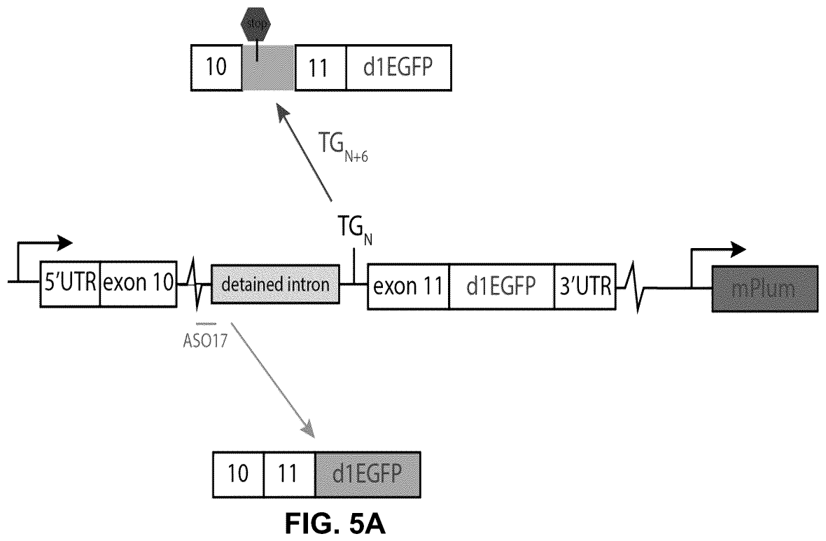

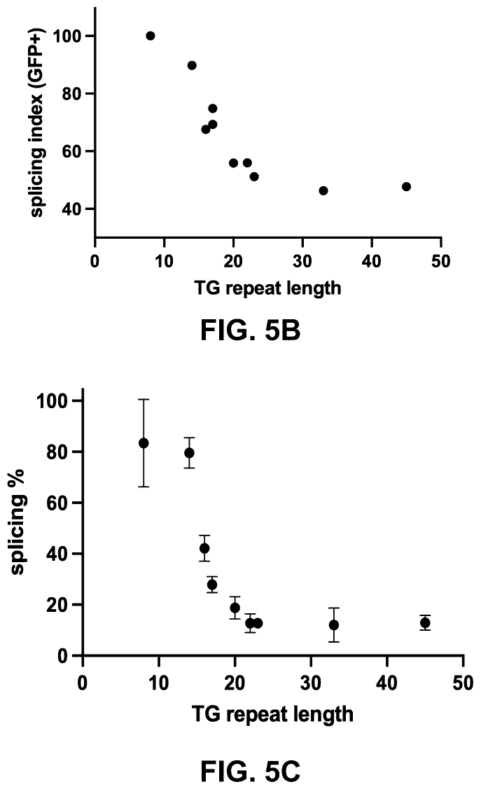

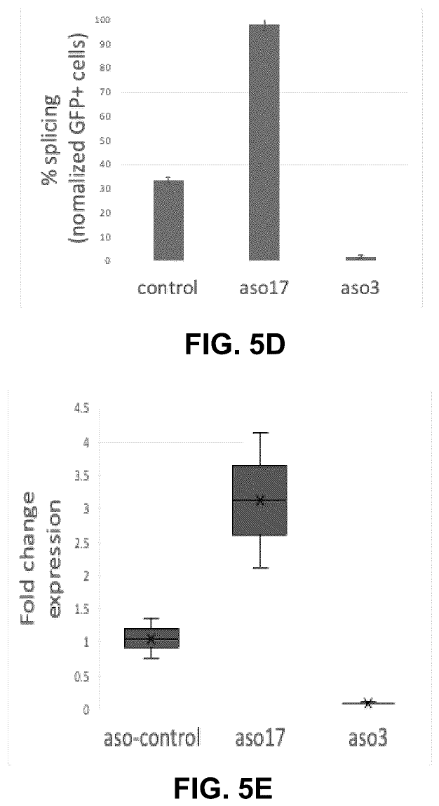

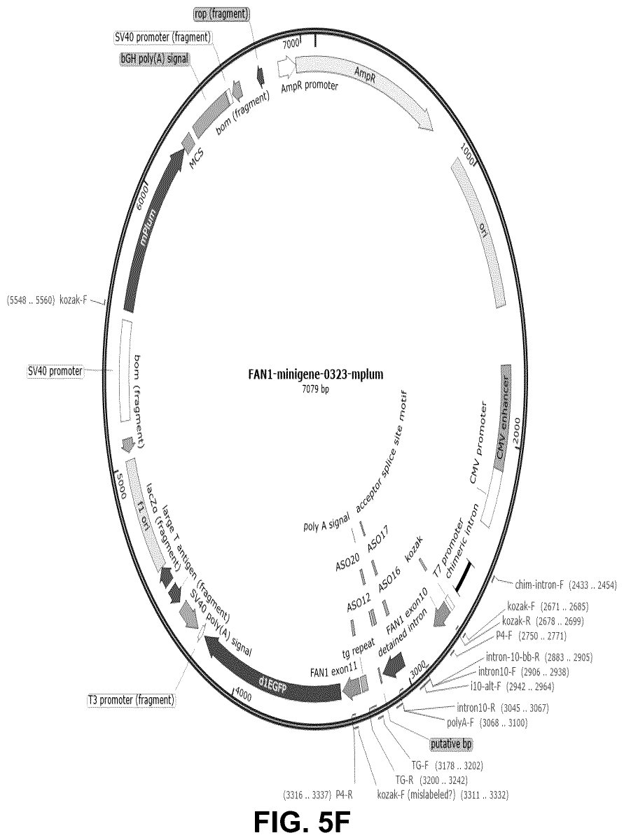

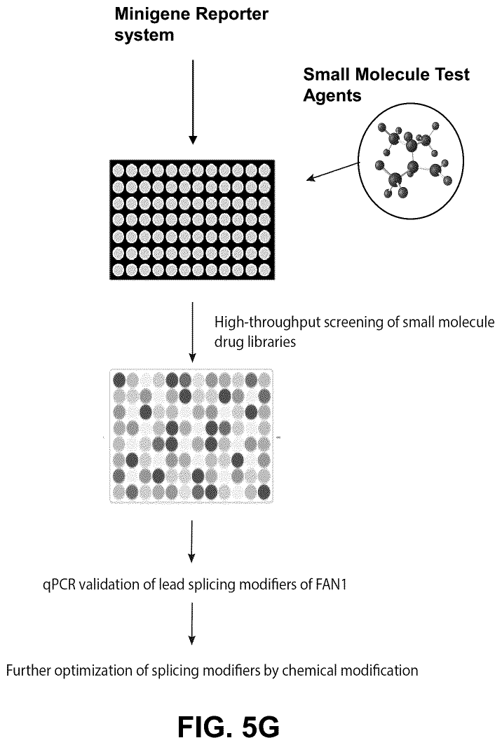

A minigene EGFP reporter system including a segment of the FAN1 exon 10-intron 10-exon 11 junction (SEQ ID NOs:1-3) that contains the variable dinucleotide repeat has been developed (as depicted in the vector map of FIG. 5F). The FAN1 exon10-intron10-exon11 splice junction is linked to a d1EGFP reporter, requiring splicing out of intron10 for fluorescence. A mPlum reporter gene (mPlum) serves as a transfection control.

As shown in the Examples, the minigene reporter system is suited for screening for splice switching ASOs that can result in efficient splicing of the FAN1 exon10-exon11 junction to produce desirable and functional transcript (FIG. 5A).

Compositions of minigene reporter system are provided. In some embodiments, the minigene reporter system include the specific region of interest in FAN1 mRNA, for example, exon 10-intron 10-exon 11 junction (SEQ ID NOs:1-3), and a reporter gene, for example EGFP.

The minigene reporter systems enable high-throughput screening of small molecules and splicing modulators to discover additional therapeutics to modulate FAN1 expression in the treatment of disorders associated with FAN1 activity.

In one embodiment, the minigene reporter system has the nucleotide sequence of SEQ ID NO. 14:

(SEQ ID NO: 14)
TGGGTGCACGAGTGGGTTACATCGAACTGGATCTCAACAGCGGTAAGATCCTT
GAGAGTTTTCGCCCCGAAGAACGTTTTCCAATGATGAGCACTTTTAAAGTTCTGCTATGTGGC
GCGGTATTATCCCGTATTGACGCCGGGCAAGAGCAACTCGGTCGCCGCATACACTATTCTCAG
AATGACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACAACG
ATCGGAGGACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCACGATGCCT
GTAGCAATGGCAACAACGTTGCGCAAACTATTAACTGGCGAACTACTTACTCTAGCTTCCCGG
CAACAATTAATAGACTGGATGGAGGCGGATAAAGTTGCAGGACCACTTCTGCGCTCGGCCCTT
CCGGCTGGCTGGTTTATTGCTGATAAATCTGGAGCCGGTGAGCGTGGGTCTCGCGGTATCATT
GCAGCACTGGGGCCAGATGGTAAGCCCTCCCGTATCGTAGTTATCTACACGACGGGGAGTCAG
GCAACTATGGATGAACGAAATAGACAGATCGCTGAGATAGGTGCCTCACTGATTAAGCATTGG
TAACTGTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGTGAGTTT
TCGTTCCACTGAGCGTCAGACCCCGTAGAAAAGATCAAAGGATCTTCTTGAGATCCTTTTTTT
CTGCGCGTAATCTGCTGCTTGCAAACAAAAAAACCACCGCTACCAGCGGTGGTTTGTTTGCCG
GATCAAGAGCTACCAACTCTTTTTCCGAAGGTAACTGGCTTCAGCAGAGCGCAGATACCAAAT
ACTGTTCTTCTAGTGTAGCCGTAGTTAGGCCACCACTTCAAGAACTCTGTAGCACCGCCTACA
TACCTCGCTCTGCTAATCCTGTTACCAGTGGCTGCTGCCAGTGGCGATAAGTCGTGTCTTACC
TGGTTGGACTCAAGACGATAGTTACCGGATAAGGCGCAGCGGTCGGGCTGAACGGGGGGTTCG
TGCACACAGCCCAGCTTGGAGCGAACGACCTACACCGAACTGAGATACCTACAGCGTGAGCTA
TGAGAAAGCGCCACGCTTCCCGAAGGGAGAAAGGCGGACAGGTATCCGGTAAGCGGCAGGGTC
GGAACAGGAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGGAGCCTA
TGGAAAAACGCCAGCAACGCGGCCTTTTTACGGTTCCTGGCCTTTTGCTGGCCTTTTGCTCAC
ATGGCTCGACAGATCTTCAATATTGGCCATTAGCCATATTATTCATTGGTTATATAGCATAAA
TCAATATTGGCTATTGGCCATTGCATACGTTGTATCTATATCATAATATGTACATTTATATTG
GCTCATGTCCAATATGACCGCCATGTTGGCATTGATTATTGACTAGTTATTAATAGTAATCAA
TTACGGGGTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTATGTTCCCA
TAGTAACGCCAATAGGGACTTTCCATTGACGTCAATGGGTGGAGTATTTACGGTAAACTGCCC
ACTTGGCAGTACATCAAGTGTATCATATGCCAAGTCCGCCCCCTATTGACGTCAATGACGGTA
AATGGCCCGCCTGGCATTATGCCCAGTACATGACCTTACGGGACTTTCCTACTTGGCAGTACA
TCTACGTATTAGTCATCGCTATTACCATGGTGATGCGGTTTTGGCAGTACACCAATGGGCGTG
GATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTGCGATCGCCCGCCCCGTT
GACGCAAATGGGCGGTAGGCGTGTACGGTGGGAGGTCTATATAAGCAGAGCTCGTTTAGTGAA
CCGTCAGATCACTAGAAGCTTTATTGCGGTAGTTTATCACAGTTAAATTGCTAACGCAGTCAG
TGCTTCTGACACAACAGTCTCGAACTTAAGCTGCAGTGACTCTCTTAAGGTAGCCTTGCAGAA
GTTGGTCGTGAGGCACTGGGCAGGTAAGTATCAAGGTTACAAGACAGGTTTAAGGAGACCAAT
AGAAACTGGGCTTGTCGAGACAGAGAAGACTCTTGCGTTTCTGATAGGCACCTATTGGTCTTA
CTGACATCCACTTTGCCTTTCTCTCCACAGGTGTCCACTCCCAGTTCAATTACAGCTCTTAAG
GCTAGAGTACTTAATACGACTCACTATAGGCTAGCGCGCCACCATGGTGTTTGTGATGGAGGC
CGGGGAGGCCGCTGACCCCACCACGGTCCTGTGCTCTGTGGAGGAGCTGGCACTGGCCCATTA
CAGACGCAGCGGTTTTGACCAGGGTAACTGAGCAGGCTTTCTCTCGTGGCACCCAGCCCCGGG
TGGACGAGCAGCAGCACTGGATGGGCTGTCAGCAGTGGGCTGCTGTCCTCTCTCTGTCCTCTG
CTCACAGTGGAAGATGCCTATAGAAAGACAACCACAAGATAGGAGTTTACAAAGCCCAGGTTC
TTTCTGATGAGTGTTGAGATTCATCTACATAATATCTTTATTACTTACAGGGAAGGGTGATGT
TATTGGGGAAGACGTGCAAATGCCAGTGAAGACAGAGAGATACAGTAGGCCTGGCCCTTAAGG
CTCTGTATATAAACAACATACTGTATAAAATAAACTGGGAATGGCGTGAAAACAGCATTTGCT
TCTGAATCTAAAATATTTCTATTATTTTCTTGAAATTGAGTGTGCAGTAGGTTATGGTGGTGT
TTTGGAAGAAACAGATAAAACAGATTTTGTGTGTGTGTGTGTGTGTGTGTGTGTGTGTGTGAC
CTTGTCTTAGGGATTCATGGCGAAGGGTCCACCTTCAGCACCCTGTATGGCCTCCTCCTGTGG
GACATCATCTTCATGGATGGGATTCCGGATGTCTTCAGAAACGCCTGTGGTGGTGGTTCCATG
GTGAGCAAGGGCGAGGAGCTGTTCACCGGGGTGGTGCCCATCCTGGTCGAGCTGGACGGCGAC
GTAAACGGCCACAAGTTCAGCGTGTCCGGCGAGGGCGAGGGCGATGCCACCTACGGCAAGCTG
ACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTC
AAGTCCGCCATGCCCGAAGGCTACGTCCAGGAGCGCACCATCTTCTTCAAGGACGACGGCAAC
TACAAGACCCGCGCCGAGGTGAAGTTCGAGGGCGACACCCTGGTGAACCGCATCGAGCTGAAG
GGCATCGACTTCAAGGAGGACGGCAACATCCTGGGGCACAAGCTGGAGTACAACTACAACAGC
CACAACGTCTATATCATGGCCGACAAGCAGAAGAACGGCATCAAGGTGAACTTCAAGATCCGC
CACAACATCGAGGACGGCAGCGTGCAGCTCGCCGACCACTACCAGCAGAACACCCCCATCGGC
GACGGCCCCGTGCTGCTGCCCGACAACCACTACCTGAGCACCCAGTCCGCCCTGAGCAAAGAC
CCCAACGAGAAGCGCGATCACATGGTCCTGCTGGAGTTCGTGACCGCCGCCGGGATCACTCTC
GGCATGGACGAGCTGTACAAGAAGCTTAGCCATGGCTTCCCGCCGGCGGTGGCGGCGCAGGAT
GATGGCACGCTGCCCATGTCTTGTGCCCAGGAGAGCGGGATGGACCGTCACCCTGCAGCCTGT
GCTTCTGCTAGGATCAATGTGTAGGCGGCCGCTTCCCTTTAGTGAGGGTTAATGCTTCGAGCA
GACATGATAAGATACATTGATGAGTTTGGACAAACCACAACTAGAATGCAGTGAAAAAAATGC
TTTATTTGTGAAATTTGTGATGCTATTGCTTTATTTGTAACCATTATAAGCTGCAATAAACAA
GTTAACAACAACAATTGCATTCATTTTATGTTTCAGGTTCAGGGGGAGATGTGGGAGGTTTTT
TAAAGCAAGTAAAACCTCTACAAATGTGGTAAAATCCGATAAGGATCGATCCGGGCTGGCGTA
ATAGCGAAGAGGCCCGCACCGATCGCCCTTCCCAACAGTTGCGCAGCCTGAATGGCGAATGGA
CGCGCCCTGTAGCGGCGCATTAAGCGCGGCGGGTGTGGTGGTTACGCGCAGCGTGACCGCTAC
ACTTGCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTTACG
GCACCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAAAC
TGGAACAACACTCAACCCTATCTCGGTCTATTCTTTTGATTTATAAGGGATTTTGCCGATTTC
GGCCTATTGGTTAAAAAATGAGCTGATTTAACAAAAATTTAACGCGAATTTTAACAAAATATT
AACGCTTACAATTTCCTGATGCGGTATTTTCTCCTTACGCATCTGTGCGGTATTTCACACCGC
ATACGCGGATCTGCGCAGCACCATGGCCTGAAATAACCTCTGAAAGAGGAACTTGGTTAGGTA
CCTTCTGAGGCGGAAAGAACCAGCTGTGGAATGTGTGTCAGTTAGGGTGTGGAAAGTCCCCAG
GCTCCCCAGCAGGCAGAAGTATGCAAAGCATGCATCTCAATTAGTCAGCAACCAGGTGTGGAA
AGTCCCCAGGCTCCCCAGCAGGCAGAAGTATGCAAAGCATGCATCTCAATTAGTCAGCAACCA
TAGTCCCGCCCCTAACTCCGCCCATCCCGCCCCTAACTCCGCCCAGTTCCGCCCATTCTCCGC
CCCATGGCTGACTAATTTTTTTTATTTATGCAGAGGCCGAGGCCGCCTCGGCCTCTGAGCTAT
TCCAGAAGTAGTGAGGAGGCTTTTTTGGAGGCCTAGGCTTTTGCAAAAAGCTTGCCACAACCC
GGGATCCACCGGTCGCCACCATGGTGAGCAAGGGCGAGGAGGTCATCAAGGAGTTCATGCGCT
TCAAGGAGCACATGGAGGGCTCCGTGAACGGCCACGAGTTCGAGATCGAGGGCGAGGGCGAGG
GCCGCCCCTACGAGGGCACCCAGACCGCCAGGCTGAAGGTGACCAAGGGTGGCCCCCTGCCCT
TCGCCTGGGACATCCTGTCCCCTCAGATCATGTACGGCTCCAAGGCCTACGTGAAGCACCCCG
CCGACATCCCCGACTACTTGAAGCTGTCCTTCCCCGAGGGCTTCAAGTGGGAGCGCGTGATGA
ACTTCGAGGACGGCGGCGTGGTGACCGTGACCCAGGACTCCTCCCTGCAGGACGGCGAGTTCA
TCTACAAGGTGAAGGTGCGCGGCACCAACTTCCCCTCCGACGGCCCCGTAATGCAGAAGAAGA
CCATGGGCTGGGAGGCCTCCTCCGAGCGGATGTACCCCGAGGACGGCGCCCTGAAGGGCGAGA
TGAAGATGAGGCTGAGGCTGAAGGACGGCGGCCACTACGACGCCGAGGTCAAGACCACCTACA
TGGCCAAGAAGCCCGTGCAGCTGCCCGGCGCCTACAAGACCGACATCAAGCTGGACATCACCT
CCCACAACGAGGACTACACCATCGTGGAACAGTACGAGCGCGCCGAGGGCCGCCACTCCACCG
GCGCCAGATCTCGAGCTCAAGCTTCGAATTCTGCAGTCGACGGTACCGCGGGCCCGGGATCCA
CCGGATCTAGATAACTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAG
GACAGCAAGGGGGAGGATTGGGAAGACAATAGCAGGCATGCTGGGGATGCGGTGGGCTCTATG
GCTTCTGAGGCGGAAAGAACCAGCTGGGGCAGATCCGCGTATGGTGCACTCTCAGTACAATCT
GCTCTGATGCCGCATAGTTAAGCCAGCCCCGACACCCGCCAACACCCGCTGACGCGCCCTGAC
GGGCTTGTCTGCTCCCGGCATCCGCTTACAGACAAGCTGTGACCGTCTCCGGGAGCTGCATGT
GTCAGAGGTTTTCACCGTCATCACCGAAACGCGCGAGACGAAAGGGCCTCGTGATACGCCTAT
TTTTATAGGTTAATGTCATGATAATAATGGTTTCTTAGACGTCAGGTGGCACTTTTCGGGGAA
ATGTGCGCGGAACCCCTATTTGTTTATTTTTCTAAATACATTCAAATATGTATCCGCTCATGA
GACAATAACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCAGAAA
CGCTGGTGAAAGTAAAAGATGCTGAAGATCAGT

1. Integration of Fan1 Minigene Splicing Reporter with Splicerush Platform

In some forms, the described compositions of minigene reporter systems are configured for integration within one or more other platforms. For example, in some forms, the described compositions of minigene reporter systems are configured for integration within one or more other platforms to identify novel splicing regulatory elements (SREs) within the FAN1 gene, particularly focusing on intron retention and cryptic splicing regions.

i. SpliceRush Intergration

In some forms, the described compositions of minigene reporter systems are configured for integration within the SpliceRush platform, utilizing the dCasRx system for high-throughput screening of splicing events.

The SpliceRush platform (Recinos, et al., Nat Commun. 2024; 15: 3839), utilizes guide RNAs to target splicing regulatory elements and modulate splicing events within a native sequence context. This system allows precise manipulation of splicing mechanisms using a combination of guide RNAs and the dCasRx system, which specifically targets RNA sequences without cutting, thus modifying splicing outcomes.

In some forms, the FAN1 minigene splicing reporter is integrated into the SpliceRush platform. This reporter system includes key exon-intron junctions critical for FAN1 splicing, linked to dual fluorescent reporters (GFP and TdTomato) for direct visualization of splicing outcomes. The fluorescence-based detection methods enable rapid assessment of splicing modulation, with high-throughput screening capabilities provided by the SpliceRush platform.

In some forms, the FAN1 minigene splicing reporter includes a minigene construct that includes FAN1 exon10-intron10-exon11 splice junctions, linked to GFP and TdTomato reporters. Proper splicing of this minigene results in GFP and/or TdTomato expression, serving as a direct readout of splicing events.

The SpliceRush platform utilizes dCasRx, a catalytically inactive variant of CasRx, to guide RNA molecules to specific splicing regulatory elements within the FAN1 gene. This system allows for the high-throughput interrogation of these elements and identification of novel SREs.

In some forms, the SpliceRush platform facilitates large-scale screening of guide RNA libraries targeting various splicing junctions within FAN1. Flow cytometry and fluorescence-based detection are used to sort cells based on GFP and TdTomato expression levels, enabling sequencing-based enrichment of gRNAs to identify effective SREs.

In some forms, a FAN1 minigene splicing reporter includes a construct enabling the visualization of splicing events within the FAN1 gene through GFP and tdtomato expression, used in combination with the splicerush platform.

In some forms, a FAN1 minigene splicing reporter identifies SRES by the integration of the splicerush platform to allow for the high-throughput identification of SRES within FAN1, focusing on regions involved in intron retention and cryptic splicing.

In some forms, a FAN1 minigene splicing reporter implements the dCasRx system for precise targeting and modulation of RNA sequences within the FAN1 gene, enabling detailed study and manipulation of splicing events.

In some forms, a FAN1 minigene splicing reporter implements dual fluorescent reporters, for example, employing GFP and tdtomato as reporters for splicing outcomes, allowing for rapid and efficient screening of splicing regulatory elements. Sorting based on fluorescence levels using flow cytometry enables sequencing-based enrichment of gRNAs to identify SRES in a high-throughput manner.

### III. Methods of Use

Methods of using the described compositions including Anti-sense oligonucleotides (ASO) that target FAN1, splice-switching ASOs (SSASO) that target FAN1, and vectors for the enhanced expression of FAN1, as well as mini-gene constructs for assaying molecular agents that modulate expression of the pathological TG-repeats within FAN1 are provided. In some forms, the methods described herein are useful in the treatment of disorders associated with FAN1 activity in a subject in need thereof.

### A. Methods of Treatment

Methods of treating, ameliorating or delaying the onset of a trinucleotide repeat expansion disorder in a subject in need thereof are provided. In some embodiments, the compositions and methods described herein are useful in the treatment of disorders associated with FAN1 activity.

Typically, an effective amount of the disclosed composition is administered to an individual in need thereof. The composition or formulation thereof may also include a targeting agent for delivery to a specific cell type.

In some embodiments, the methods including a step of selecting a subject who is likely to benefit from treatment with the disclosed compositions.

ASO-based gene modulation mechanisms that result in decreased expression of unwanted genes or increased gene expression (including use of SSASO) are known in the art. Typically, in the case of mammals, Gdna in the nucleus is transcribed to pre-Mrna. An exogenous ASO in the nucleus hybridizes A) to the 3′-most polyadenylation signal on the pre-Mrna and blocks polyadenylation at this site, thereby redirecting it to another site upstream, which upregulates gene expression1 B) to a splice site, thereby preventing proper assembly of the spliceosome, which leads to exon skipping and therefore improved expression of a disease gene2 (not considered a true ASO by many, these are often called splice switching oligonucleotides [SSO] or more generally, steric blocking oligonucleotides [SBO]) C) an exon or intron (in this case, an intron), thereby leading to cleavage by Rnase H3. In most cases, though significantly upregulated, silenced, or altered, some processing of the unaffected pre-Mrna is likely to occur followed by export of the mature Mrna to the cytoplasm.

### B. Individuals to Be Treated

A subject in need of treatment is a subject having or at risk of developing one or more trinucleotide repeat disorders. In preferred embodiments, the disclosed compositions and methods thereof are effective in ameliorating or delaying the onset of one or more symptoms associated with a trinucleotide repeat expansion disorder.

Trinucleotide repeat disorders consist of a group of human diseases, which are a result of an abnormal expansion of repetitive sequences and primarily affect the nervous system. These occur during various stages of human development. Repetitive sequences, scattered in the microsatellite regions, usually account for about 30% of the human genome. In a normal person, the main purpose of various lengths of repetitive DNA is to allow for evolutionary plasticity. However, when these repeats extend beyond the code for a viable physiological protein, the expression of this aberrant segment is suppressed. After a certain threshold number, this suppression is lost, and an aberrant protein is coded for, which gives rise to either a functional or a non-functional protein, thereby giving rise to a ‘gain of function’ or ‘loss of function’ mutation. With every generation, the number of repeats increases drastically, and the age at which the patient presents is inversely related to the number of expansions. The severity, on the other hand, worsens with every generation due to a larger repeat sequence.

Trinucleotide repeat disorders were classified as type 1, which are polyglutamate (polyQ) disorders with abnormal CAG repeats in the coding region, and type 2 or non-polyglutamate (non-polyQ) disorders which are triplet expansions in the non-coding regions, by La Spada et al. (Shetty K T, Christopher R. Indian J Clin Biochem. 2000 August; 15(Suppl 1):136-44). Myotonic dystrophy (DM), Huntington disease, spinocerebellar ataxia, Friedreich ataxia, and fragile X syndrome fall under the spectrum of trinucleotide repeat disorders (Paulson H. Handb Clin Neurol. 2018; 147:105-123).

In some embodiments, the disclosed compositions and methods thereof are effective in ameliorating or delaying the onset of one or more symptoms associated with myotonic dystrophy (DM), Huntington disease, spinocerebellar ataxia, Friedreich ataxia, and fragile X syndrome in a subject in need thereof.

### C. Methods of Administration

A number of different delivery paradigms have been utilized to deliver SSOs to cells in vivo, including intraperitoneal (IP), subcutaneous (SC) or intravenous (IV) administration. These methods result in exposure of many peripheral tissues to the oligonucleotide (Geary R. S., et al., Adv. Drug Deliv. Rev. 2015; 87:46-51; Hua Y et al., Nature. 2011; 478:123-126; Hung G et al., Nucleic Acid Ther. 2013; 23:369-378). Other approaches, such as intramuscular (IM), intratumoral (ITM), subconjunctival (SCJ) or intravitreal (IVI) injection of ASOs have been used to achieve more tissue-specific delivery. ASOs do not readily cross the blood brain barrier when administered peripherally (Geary R. S. Expert Opin. Drug Metab. Toxicol. 2009; 5:381-391). However, for therapeutics intended for CNS applications and targets, direct delivery to the cerebrospinal fluid (CSF) by either intracerebroventricular (ICV) or intrathecal (IT) administration has been shown to result in therapeutic doses of SSOs throughout the CNS, though deeper brain regions are more challenging to access (Smith R. A. et al., J. Clin. Invest. 2006; 116:2290-2296).

The compositions are generally administered to a subject in an effective amount. As used herein the term “effective amount” means a dosage sufficient to inhibit or delay one or more symptoms of a disease or to otherwise provide a desired pharmacologic and/or physiologic effect. The precise dosage will vary according to a variety of factors such as subject-dependent variables (e.g., age, immune system health, etc.), the specific variant of virus, and the treatment being affected.

The pharmaceutical compositions can be for administration by parenteral (intramuscular, intraperitoneal, intravenous, or subcutaneous injection) or using bioerodible inserts and can be formulated in dosage forms appropriate for each route of administration.

In some embodiments, the compositions are administered locally. For example, directly in the brain tissue, by direct infusion or intracranial convection-enhanced delivery (CED). In other embodiments, the compositions are administered via ICV (intracerebroventricular), IS (intrastriatal), IT (intrathecal), ITM (intratumoral), IVI (intravitreal), or SCJ (subconjunctival) route of administration. Typically, local administration causes an increased localized concentration of the compositions which is greater than that which can be achieved by systemic administration. In some embodiments, the compositions are delivered locally to the appropriate cells by using a syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

### D. Dosage and Effective Amounts

Dosage and dosing regimens are dependent on the severity and location of the disorder or injury and/or methods of administration, as well as the therapeutic or prophylactic agent being delivered. This can be determined by those skilled in the art. A therapeutically effective amount of the disclosed composition used in the treatment of one or more trinucleotide repeat disorders is typically sufficient to ameliorating or delaying onset of one or more symptoms associated with the disorder.

Preferably, the therapeutic, prophylactic or diagnostic agents do not target or otherwise modulate the activity or quantity of healthy cells not within or associated with the diseased/damaged tissue, or do so at a reduced level compared to cells associated with a disease or disorder such as damaged neurons and brain tissues in the case of Huntington's disease. In this way, by-products and other side effects associated with the compositions are reduced. Therefore, in preferred embodiments, the compositions are administered in an amount that leads to an improvement, or enhancement, function in an individual with a disease or disorder.

The actual effective amounts of the composition can vary according to factors including the specific agent administered, the particular composition formulated, the mode of administration, and the age, weight, condition of the subject being treated, as well as the route of administration and the disease or disorder. Generally, for intravenous injection or infusion, the dosage will be lower than for oral administration.

Dosage can vary and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill in the art can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

Dosage forms of the pharmaceutical composition including the compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly, or yearly dosing.

It will be understood by those of ordinary skill that a dosing regimen can be any length of time sufficient to treat the disorder in the subject.

### E. Combination Therapies

The disclosed compositions can be administered alone or in combination with one or more existing therapies and clinical management. In some embodiments, the combination results in an additive effect on the treatment of the disease or condition. In some embodiments, the combinations result in a more than additive effect on the treatment of the disease or disorder.

In some embodiments, the composition is administered prior to, in conjunction with, subsequent to, or in alternation with, treatment with one or more additional therapies or procedures. In some embodiments, the additional therapy is performed between drug cycles or during a drug holiday that is part of the composition dosage regime.

In the context of Huntington's chorea, the current management of Huntington's mainly aims at managing each symptom rather than the basic pathogenesis. More recently, however, clinicians have tended first to use newer atypical antipsychotic drugs in persons who experience severe chorea, especially when it is accompanied by psychiatric symptoms warranting antipsychotic use, such as delusions. Depression, which commonly accompanies Huntington's Disease, is treated with newer antidepressants. Thus far, limited trials of cognitive-enhancing agents used primarily in patients with AD, such as memantine, rivastigmine, and donepezil, have shown only modest benefit. Bradykinesia and rigidity in younger-onset individuals can respond to dopaminergic agents used in parkinsonism.

Treatment for Huntington's disease can include a dopamine blocker to help reduce abnormal behaviors and movements, or a drug such as amantadine and tetrabenazine to control movement, etc. Other drugs that help to reduce chorea include neuroleptics and benzodiazepines. Compounds such as amantadine or remacemide have shown preliminary positive results. Hypokinesia and rigidity, especially in juvenile cases, can be treated with antiparkinsonian drugs, and myoclonic hyperkinesia can be treated with valproic acid. Psychiatric symptoms can be treated with medications similar to those used in the general population. Selective serotonin reuptake inhibitors and mirtazapine have been recommended for depression, while atypical antipsychotic drugs are recommended for psychosis and behavioral problems.

In the context of Friedrich's ataxia, antioxidants are biological and chemical compounds that reduce oxidative damage. The following are the antioxidants commonly used in Friedrich's patients-Coenzyme Q, Vitamin E, High dose ascorbic acid, Idebenone a synthetic coenzyme Q, N-acetylcysteine, selegiline, Dehydroepiandrosterone, Pioglitazone, a peroxisome proliferator-activated receptor-gamma (PPARγ) and induces the expression of enzymes involved in mitochondrial metabolism, including superoxide dismutase, which is an important antioxidant defense in nearly all cells exposed to oxygen (Kearney M, Orrell R W, Fahey M, Brassington R, Pandolfo M. Cochrane Database Syst Rev. 2016 Aug. 30; 2016(8):CD007791).

In the context of Fragile X syndrome, careful medical follow up, and sometimes intervention is required as the physical and behavioral problems of fragile X patients are related to their stage of development. Neuropsychiatric manifestations: Seizures are observed in approximately 20% of males and 5% of females and necessitate a timely diagnosis and treatment. Influencing behavioral problems is difficult, although behavioral therapy and avoidance of overwhelming stimuli may alleviate some of the symptoms. Some physicians recommend pharmacological intervention for behavioral problems. The need for special education and training, especially in younger children, is of primary importance. Speech therapists and physiotherapists can help with language and motor development. Connective tissue manifestations: During infancy, associated connective tissue abnormalities may present as congenital hip dislocations and inguinal hernia, that need surgical correction. Some children fail to thrive because of gastro-oesophageal reflux, tactile defensiveness, or difficulties in sucking. The latter of these problems requires attention from a specialized speech therapist or physiotherapist, while the gastro-oesophageal reflux can be treated by dietary advice or medication or both. Other: The frequent otitis media and sinusitis in approximately 50% of affected children require adequate intervention (antibiotics or polyethylene tubes or both). Approximately 30-50% of cases need ophthalmological help for strabismus, myopia, or hyperopia (de Vries B B, Halley D J, Oostra B A, Niermeijer M F. J Med Genet. 1998 July; 35(7):579-89).

In the context of Spinocerebellar ataxia, no current FDA approved treatment exists for SCA. However, gene testing can confirm the diagnosis. Although incurable, establishing a specific diagnosis can put an end to the quest for the etiology, permit a discussion of the prognosis, and facilitate discussions of genetic risk to other family members. Further advancements in treatment are currently in the pipeline. A 24-week neuro-rehabilitation program with neuro-rehabilitation therapy, focusing on balance, coordination, and muscle strengthening, has been found to be beneficial for reducing cerebellar symptoms in spinocerebellar ataxia type-2. The relevance in other types of SCA is being studied in detail.

In the context of Muscular dystrophy, the management is based on genetic counseling, preserving function and independence, and preventing complications. In DM1, the combined effects of disordered breathing and weakness of the diaphragm and oropharyngeal muscles often lead to respiratory impairment and nocturnal hypoventilation. It is useful to monitor FVC and FEV1 changes from sitting to a supine position at clinic visits. Many patients will progress to the point of requiring non-invasive nighttime ventilatory support. Placement of a pacemaker or cardiac defibrillator can be lifesaving in DM1. The ECG should be monitored annually (Thornton CA. Myotonic dystrophy. Neurol Clin. 2014 August; 32(3):705-19, viii).

The described compositions and methods will be better understood in view of the following numbered paragraphs.

1. A method of ameliorating or delaying the onset of a trinucleotide repeat expansion disorder in a subject in need thereof including increasing the level of mRNA and/or protein expression of FANCD2 And FANCI Associated Nuclease 1 (FAN1), wherein the subject has more than 17 dinucleotide repeats of thymine and guanine (TG) in intron 10 of FAN1 pre-mRNA.

2. The method of Paragraph 1, wherein the subject has 18, 19, 20, 21, 22, 23, or more than 23 dinucleotide TG repeats in intron 10 of the FAN1 pre-mRNA.

3. The method of any one of Paragraphs 1-2, wherein the subject has a reduced level of mRNA and/or protein expression of FAN1 compared to a healthy control.

4. The method of any one of Paragraphs 1-3, wherein the subject has a reduced efficiency of splicing in exon10 and exon11 junction in the pre-mRNA of FAN1 to produce functional mRNA transcripts of FAN1.

5. The method of any one of Paragraphs 1-4, wherein the step of increasing the level of mRNA and/or protein expression of FAN1 includes administering to the subject a composition effective to increase levels of functional FAN1 mRNA transcripts encoding FAN1 derived from the pre-mRNA,
optionally, wherein the composition includes
(i) one or more splice switching antisense oligonucleotides (ASOs), and/or
a Cas enzyme and a single guide RNA (sgRNA) sequence; and
(ii) in a pharmaceutically acceptable carrier.

6. The method of Paragraph 5, wherein the ASOs specifically target the detained intron within intron 10 of FAN1 pre-mRNA.

7. The method of Paragraph 5 or 6, wherein the ASOs specifically target an mRNA sequence including the nucleic acid sequence set forth in SEQ ID NO:2.

8. The method of any one of Paragraphs 5-7, wherein the ASOs specifically target a region of between about 8 and about 50 contiguous nucleotides, inclusive, preferably between about 12 and about 30 contiguous nucleotides of SEQ ID NO:2.

9. The method of any one of Paragraphs 5-8, wherein the ASOs specifically target the 5′ end of the detained intron within intron 10 of FAN1 mRNA.

10. The method of any one of Paragraphs 5-9, wherein the ASO includes the nucleotide sequence of any one of SEQ ID NOs:7-9.

11. The method of any one of Paragraphs 5-10, wherein the ASO is chemically modified.

12. The method of any one of Paragraphs 5-11, wherein the ASO is chemically modified via phosphorothiate (PS) backbone modification, and/or modified at the 2′ sugar position selected from the group including 2′-O-methyl (2′-OMe) and 2′-O-methoxyethyl (2′-MOE).

13. The method of any one of Paragraphs 5-12, wherein the ASO is administered in an amount effective to increase the level of mRNA and/or protein expression of FAN1 in one or more neuronal cells compared to the level prior to the administration of the ASO.

14. The methods of any one of Paragraphs 1-4, wherein the step of increasing the level of mRNA and/or protein expression of FAN1 is achieved by CRISPR/Cas based gene editing.

15. The methods of Paragraph 14, wherein the CRISPR/Cas based gene editing includes Cas13 and an sgRNA.

16. The method of any one of Paragraphs 1-15, wherein the trinucleotide repeat expansion disorder is Huntington's disease, cerebrospinal ataxia, or fragile X syndrome.

17. The method of any one of Paragraphs 1-16, wherein the trinucleotide repeat expansion disorder is Huntington's disease.

18. The method of any one of Paragraphs 1-17, wherein the increasing the level of mRNA and/or protein expression of FAN1 is effective to delay the onset of one or more symptoms associated with Huntington's disease.

19. A method of ameliorating or delaying the onset of a trinucleotide repeat expansion disorder in a subject in need thereof including administering to the subject one or more splice switching antisense oligonucleotides (ASOs) in a pharmaceutically acceptable carrier, wherein the one or more splice switching splice switching ASOs are effective to increase levels of functional FAN1 mRNA transcripts encoding FAN1 derived from the pre-mRNA.

20. The method of Paragraph 19, wherein the ASOs specifically target the detained intron within intron 10 of FAN1 mRNA.

21. The method of Paragraph 19 or 20, wherein the ASOs specifically hybridize to SEQ ID NO: 2.

22. The method of any one of Paragraphs 19-21, wherein the ASOs specifically hybridize to a region of between about 8 and about 50, inclusive, preferably between about 12 and about 30 contiguous nucleotides of SEQ ID NO: 2.

23. The method of any one of Paragraphs 19-22, wherein the ASOs specifically target the 5′ end of the detained intron within intron 10 of FAN1 mRNA.

24. The method of any one of Paragraphs 19-23, wherein the ASO includes the nucleotide sequence of any one of SEQ ID NOs: 7-9.

25. The method of any one of Paragraphs 19-24, wherein the ASO is administered in an amount effective to increase the level of mRNA and/or protein expression of FAN1 in one or more neuronal cells compared to the level prior to the administration of the ASO.

26. A composition for increasing levels of functional FAN1 mRNA transcripts encoding FAN1 derived from the pre-mRNA, the composition including one or more splice switching antisense oligonucleotides (ASOs) in a pharmaceutically acceptable carrier.

27. The composition of Paragraph 26, wherein the ASO including 8 to 50 nucleotides, wherein the ASO specifically hybridizes to a target sequence of the 5′ end of the detained intron within intron 10 of FAN1 pre-mRNA.

28. The composition of Paragraph 27, wherein the ASO specifically hybridizes to a target sequence within SEQ ID NO: 2.

29. The composition of Paragraph 27 or 28, wherein the target sequence includes between about 8 and about 50, inclusive, preferably between about 12 and about 30 contiguous nucleotides of SEQ ID NO: 2.

30. The composition of any one of Paragraphs 27-29, wherein the target sequence includes the nucleotide sequence of any one of SEQ ID NOs: 4-6.

31. The composition of any one of Paragraphs 26-30, wherein the ASO includes the nucleotide sequence of any one of SEQ ID NOs: 7-9.

32. An antisense oligonucleotide (ASO) including between about 12 and 50 nucleotides, wherein the ASO specifically hybridizes to a target sequence of the 5′ end of the detained intron within intron 10 of FAN1 pre-mRNA.

33. The antisense oligonucleotide of Paragraph 32, wherein the ASO specifically hybridizes to a target sequence within SEQ ID NO: 2.

34. The antisense oligonucleotide of Paragraph 32 or 33, wherein the target sequence includes between about 8 and about 50, inclusive, preferably between about 12 and about 30 contiguous nucleotides of SEQ ID NO: 2.

35. The antisense oligonucleotide of any one of Paragraphs 32-34, wherein the target sequence includes the nucleotide sequence of any one of SEQ ID NOs: 4-6.

36. The antisense oligonucleotide of any one of Paragraphs 32-35, wherein the ASO includes the nucleotide sequence of any one of SEQ ID NOs: 7-9.

37. The antisense oligonucleotide of any one of Paragraphs 32-36, wherein the ASO is chemically modified.

38. The antisense oligonucleotide of any one of Paragraphs 32-37, wherein the ASO is chemically modified via phosphorothiate (PS) backbone modification, and/or modified at the 2′ sugar position selected from the group including 2′-O-methyl (2′-OMe) and 2′-O-methoxyethyl (2′-MOE).

39. A minigene reporter system including the nucleotide sequence of exon 10-intron 10-exon 11 junction of FAN1, and a reporter gene.

40. The minigene reporter system of Paragraph 39, wherein the nucleotide sequence of exon 10-intron 10-exon 11 junction of FAN1 includes the nucleotide sequences of SEQ ID NOs:1-3.

41. The minigene reporter system of Paragraph 39 or 40, wherein the reporter gene encodes enhanced green fluorescent protein.

42. A method for identifying one or more splice modulators that increase levels of functional FAN1 mRNA transcripts encoding FAN1 derived from the pre-mRNA in one or more neuronal cells including
i) contacting the minigene reporter system of any one of Paragraphs 39-41 and one or more candidate molecules, and
ii) determine the level of reporter gene expression, and
iii) select the candidate molecules that increase the level of reporter gene expression as suitable for increasing the level of mRNA and/or protein expression of FAN1 in one or more neuronal cells.

The described compositions and methods will be better understood in view of the following Examples.

### EXAMPLES

### Example 1: Design and Validation of a Splice Switching Antisense Oligonucleotide (ASO) Capable of Increasing Endogenous FAN1 Expression

### Materials and Methods

### Cell Culture:

The SHSY5Y cell line (ATCC) was maintained in Dulbecco's Modified Eagle Medium (DMEM) supplemented with 10% fetal bovine serum (FBS) and 1% penicillin-streptomycin. The cells were cultured in a humidified incubator at 37° C. with 5% C02.

### ASO Design and Synthesis:

The antisense oligonucleotides (ASOs) were designed to target specific mRNA sequences of interest using appropriate bioinformatics tools [cite]. The ASOs with a fully 2′-O-methoxyethyl (2′MOE) backbone and phosphorothiate modifications were synthesized and purified by GeneLink. The ASO stock solutions were prepared at a concentration of 100 μM and stored at −20° C. until further use.

### ASO Transfection:

For ASO transfection, SHSY5Y cells were seeded onto a 24-well tissue culture plate. The cells were plated in complete growth medium and incubated at 37° C. with 5% C02. Prior to transfection, the ASOs were diluted in Opti-MEM to a final concentration of 100 nM. The diluted ASOs were mixed with Mirus TransIT-oligo reagent according to the manufacturer's instructions to form ASO-lipid complexes. SHSY5Y cells were subjected to a reverse transfection approach, where the ASO-lipid complexes were added to the cells during cell plating. The cell suspension containing the ASO-lipid complexes was added to the wells, followed by gentle swirling to ensure even distribution. The cells were then incubated at 37° C. with 5% CO₂ for a desired transfection period of 24-48 hours.

### RNA Extraction:

After the desired transfection period, total RNA was extracted from the transfected cells using the Zymo Direct-zol RNA extraction kit according to the manufacturer's instructions. The concentration and purity of the extracted RNA were determined using a Qubit spectrophotometer and the AATI Fragment Analyzer.

cDNA Synthesis and Gene Expression Analysis:

For cDNA synthesis, reverse transcription of RNA was performed using the Maxima Reverse Transcriptase kit from Fisher, following the manufacturer's protocol.

For gene expression analysis, specific primers were designed for the quantification of FAN1 splicing and expression. Intron-spanning primers were used to target the exon10-exon11 junction and exon8-exon9 splice junctions of FAN1. Additionally, a control housekeeping primer set for RPLP0 was used. qRT-PCR was performed using a SYBR Green-based detection system with the specific primers. The gene expression levels were normalized to RPLP0, and the relative expression levels were calculated using the comparative CT method.

### Results

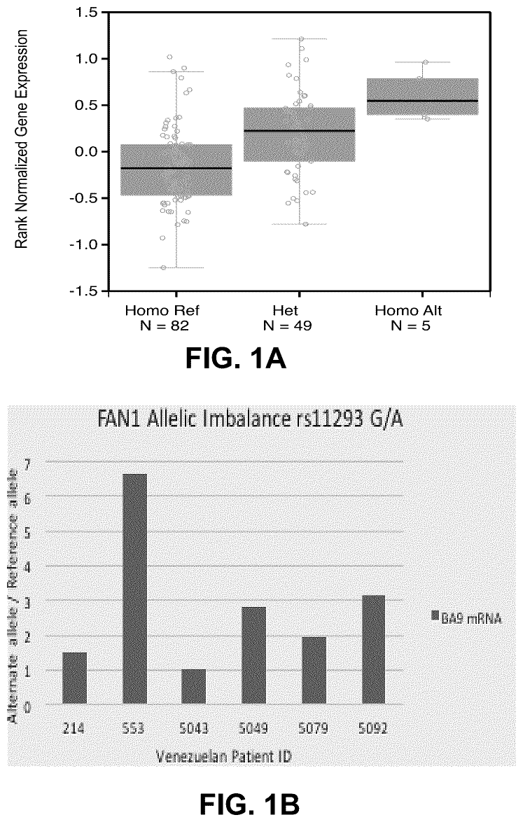

Genetic variants that delay HD patient onset are brain-specific expression quantitative trait loci (eQTLs) that increase expression of FAN1 using GTeX database and in an allelic imbalance test performed on Broadmann Area 9 cortical tissue from HD patients genotyped to be heterozygous for the FAN1 variants. (FIGS. 1A-1B).

The studies described herein are based on the hypothesis that increased FAN1 expression through the molecular mechanisms to which these variants increase FAN1 gene expression levels is therapeutically beneficial in a variety of trinucleotide repeat expansion disorders.

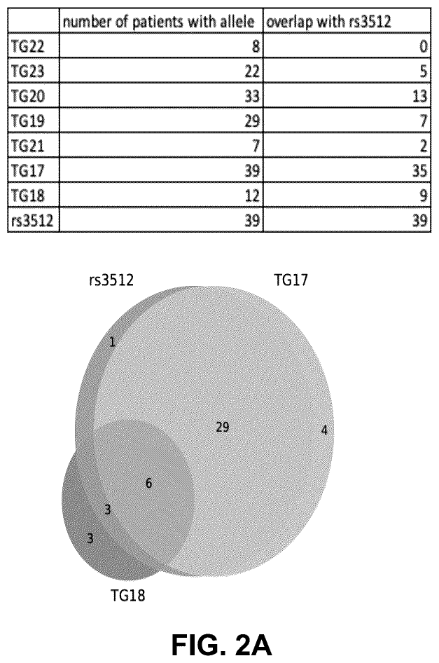

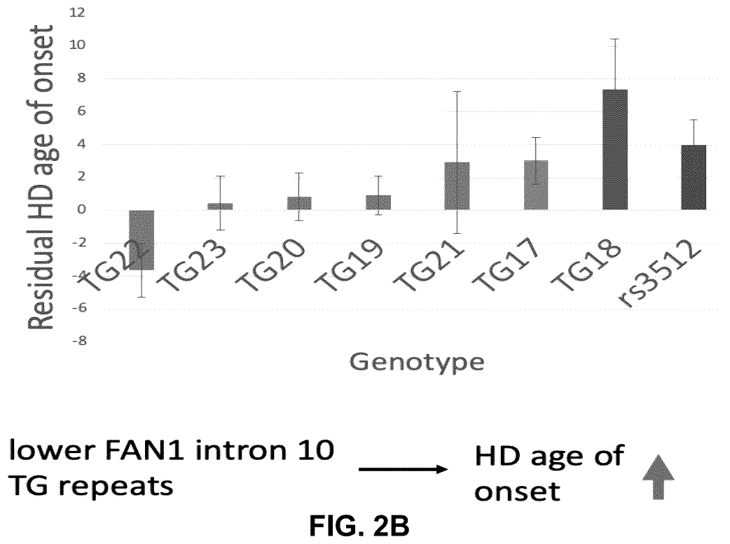

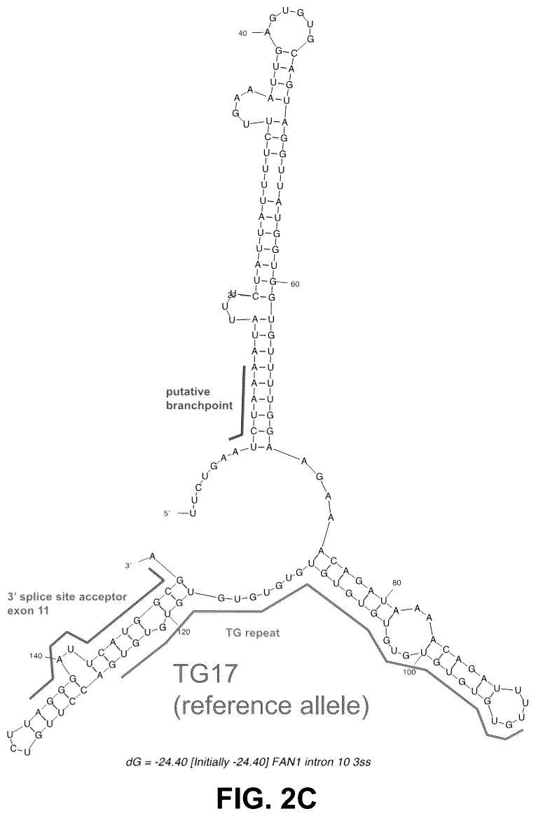

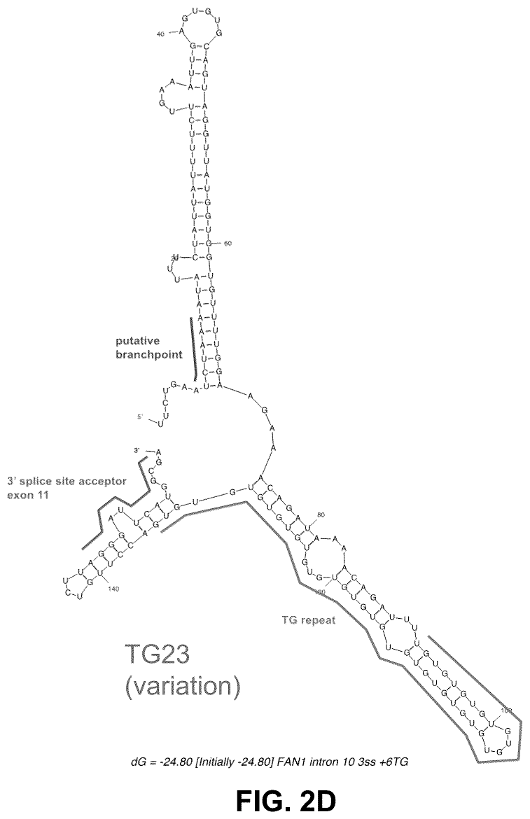

Through whole genome sequencing data on the HD Venezuelan Kindreds, the largest known repository of familial HD samples in an isolated population in which the Huntingtin (HTT) gene was first discovered, a variable TG dinucleotide repeat in intron 10 of the FAN1 gene (FIGS. 2A-2B) was discovered. It is posited that the shortest repeat found in humans (TG17) is in linkage disequilibrium with the SNPs rs11293 and rs3512 that delay HD age of onset in the FAN1 locus as opposed to long repeats that may be in linkage with the variants that hasten age of onset. The studies herein are based on the hypothesis, that longer TG repeats in the FAN1 intron leads to inefficient splicing of the FAN1 exon10-exon11 junction and that this is the functional aspect in which genetic variation in this locus leads to modification of repeat expansion disorder patient outcomes. The variable dinucleotide repeat at the 3′ splice site of FAN1 intron 10 may form stem-loop secondary structures. FIGS. 2C and 2D. Using approaches outline in FIG. 3, studies herein indeed show (through cell culture studies in the SHSY5Y neuroblastoma cell line) that longer TG repeats cause a reduction of splicing of this FAN1 junction (FIGS. 5B-5C). This variable TG repeat and associated sequences in the FAN1 exon 10-intron 10-exon 11 junction are thus a target for therapeutics to modulate FAN1 expression in the treatment of disorders associated with FAN1 activity.

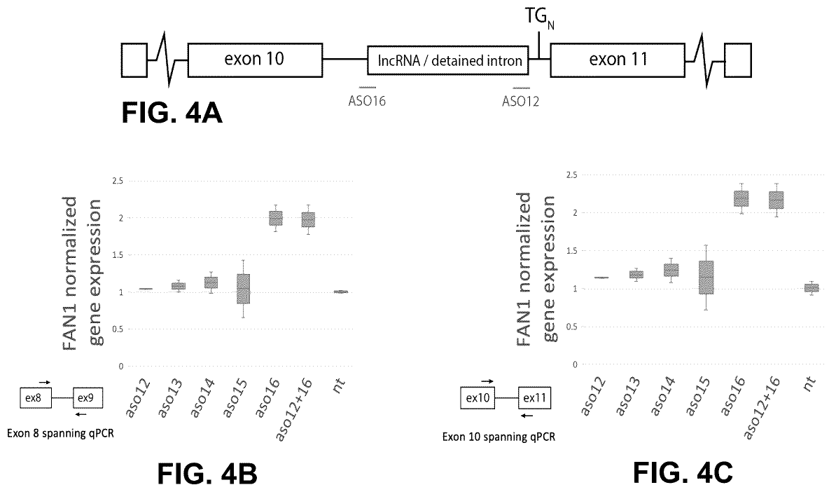

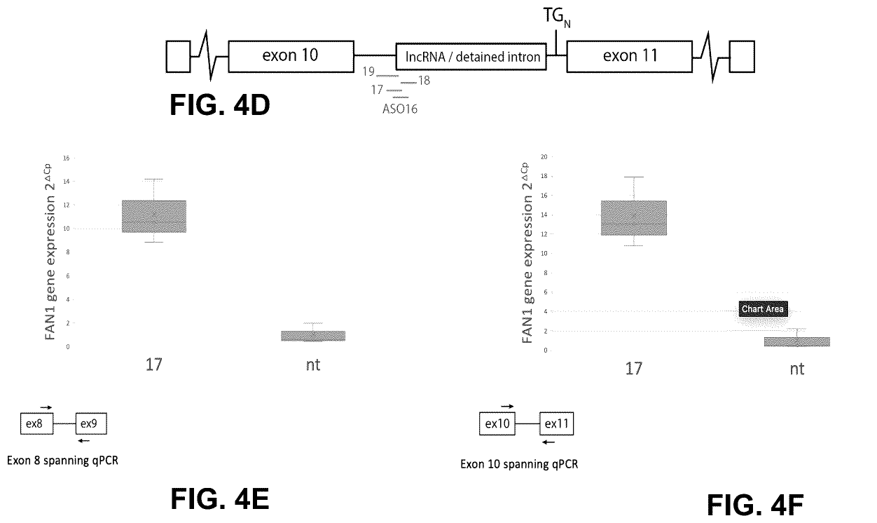

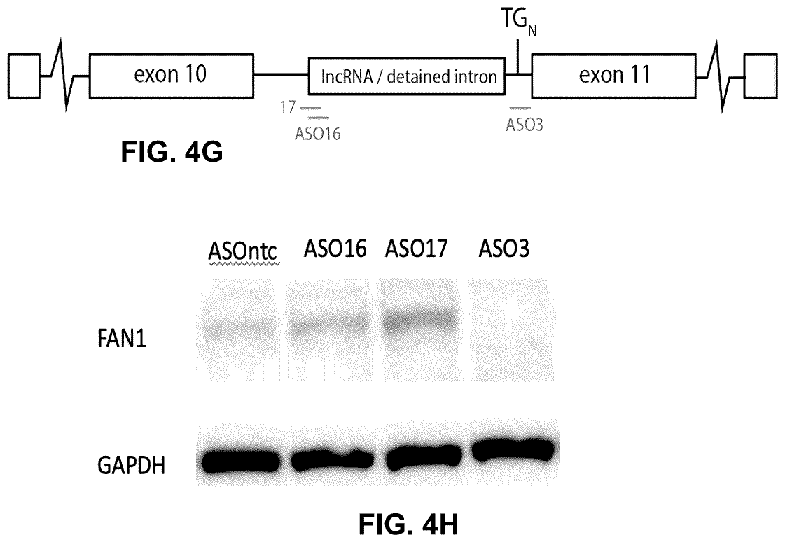

To this end, a splice switching antisense oligonucleotide (ASO) capable of increasing endogenous FAN1 expression by targeting the 5′ end of the detained intron within intron 10 of FAN1 was discovered and developed. Increased FAN1 expression at both the RNA and protein level using this ASO, was confirmed. (FIGS. 4A-4G).

### Example 2: Design and Validation of a Minigene Reporter System

A minigene EGFP reporter system was developed by cloning of the FAN1 exon 10-intron 10-exon 11 junction that contains the variable dinucleotide repeat.

### Material and Methods

### Minigene Splicing Reporter System Construction:

The FAN1 minigene was constructed using the NEB HiFi Assembly Kit, following the manufacturer's instructions. The exon10-intron10-exon11 splice junction of FAN1 was amplified from genomic DNA with a high-fidelity polymerase and cloned into the GFP reporter construct replacing the dsRed with mPlum. The construct was amplified in NEB Stable cells and purified using the Zymopure II Plasmid Miniprep and Midiprep kits.

Transfection into SHSY5Y Cells:

For transfection, SHSY5Y cells were seeded onto a 24-well tissue culture plate at a density of 100000 cells per well. The cells were plated in complete growth medium and incubated at 37° C. with 5% CO2. Prior to transfection, the plasmid DNA was diluted in Opti-MEM to a final concentration of 100 nM. The diluted DNA was mixed with Mirus TransIT-2020 reagent according to the manufacturer's instructions to form DNA-lipid complexes. SHSY5Y cells were subjected to a reverse transfection approach, where the DNA-lipid complexes were added to the cells during cell plating. The cell suspension containing the DNA-lipid complexes was added to the wells, followed by gentle swirling to ensure even distribution. The cells were then incubated at 37° C. with 5% CO2 for a desired transfection period of 24-48 hours.

Two splicing reporter constructs were designed to contain 1) the full endogenous intron 10, and 2) a shortened intron 10, which contained only the key elements necessary for splicing including the 5′ splice site and flanking sequences, 3′ splice site and flanking sequences, detained intron location, poly A sequences, branchpoint sequences, and the variable TG repeat.

### Flow Cytometry Analysis:

For flow cytometry, the BD FACSCelesta was used. The cells were detached from the culture plate using 0.25% trypsin-EDTA, resuspended in complete DMEM media, and filtered through a 35 μm filter. The cells were then analyzed by flow cytometry, gating on live cells and single cells. The data were analyzed using the BD FACSDiva software for data acquisition and FlowJo software for data analysis. The percentage of GFP+(spliced minigene) and mPlum+ (transfection control) cells was calculated.

qPCR Analysis:

To analyze the effect of different intron lengths on splicing efficiency, qPCR was performed using the Luna Universal qPCR Master Mix (https://www.neb.com/products/m3003-luna-universal-qpcr-master-mix #Product %20Information). Primers were designed to span the FAN1 exon10-exon11 junction, thereby amplifying only when splicing had occurred. The qPCR was carried out on a Roche LightCycler 480. The relative expression levels of the spliced minigene was normalized to the expression level of the mPlum transfection control.

All experiments were performed in triplicate and statistical significance was calculated using a two-tailed unpaired Student's t-test. P-values of less than 0.05 were considered significant.

### Results

Modulation of FAN1 splicing by ASOs was confirmed using this reporter system in SHSY5Y cells (FIGS. 5A-5F). FIG. 5A is a cartoon of minigene reporter system of FAN1 intron10. The exon10-intron10-exon11 splice junction of FAN1 (intron10 is truncated to contain the key splicing elements) is placed before a d1EGFP reporter. EGFP is frameshifted from the promoter such that proper protein expression and subsequent fluorescence is only achieved when the FAN1 intron10 is spliced out. An mPlum fluorescent reporter is co-expressed on an independent promoter as a transfection control. FIG. 5B shows flow cytometry results of the FAN1 exon10-exon11 junction confirming decreased splicing efficiency as the length of TG repeats increase. FIG. 5C shows qPCR of the FAN1 exon10-exon11 junction confirm decreased splicing efficiency as the length of TG repeats increase. FIG. 5D shows flow cytometry of altered splicing efficiency in the FAN1 exon10-exon11 junction when SHSY5Y cells are treated simultaneously with ASO treatments. FIG. 5E shows qPCR of altered splicing efficiency in the FAN1 exon10-exon11 junction when SHSY5Y cells are treated simultaneously with ASO treatments. FIG. 5F Plasmid map of minigene reporter system of FAN1 intron10. The exon10-intron10-exon11 splice junction of FAN1 (intron10 is truncated to contain the key splicing elements) is placed before a d1EGFP reporter. EGFP is frameshifted from the promoter such that proper protein expression and subsequent fluorescence is only achieved when the FAN1 intron10 is spliced out. An mPlum fluorescent reporter is co-expressed on an independent promoter as a transfection control.

This reporter system can therefore be used in the high-throughput screening of small molecules and splicing modulators to discover additional therapeutics to modulate FAN1 expression in the treatment of disorders associated with FAN1 activity. FIG. 5G is a cartoon showing the use of FAN1 splicing reporter to high-throughput screen small molecule drug libraries.

### Example 3: Overexpression of FAN1 by AAV.PhP.eB Retroorbital Injection in the R6/1 Mice Model of HD

### Materials and Methods

### AAV Construct Generation

The AAV constructs for FAN1 overexpression were created using Gibson assembly, as performed with the NEB HiFi Assembly kit. Individual components, including the FAN1 cDNA, AAV vector backbone, various promoters, W3SL 3′ UTR, and the miRFP670nano tag were each PCR amplified from existing plasmids. The Gibson assembly reaction was set up following the manufacturer's instructions, combining these PCR-amplified components with the linearized AAV vector. This reaction mixture was then transformed into competent E. coli cells, and positive clones were selected through colony PCR and confirmed by sequencing.

### AAV Production and Purification

Recombinant AAV vectors were produced and purified as per the protocols described by Chan et al. (Chan, et al., Nat Neurosci, 2017 August; 20(8):1172-1179). In brief, human embryonic kidney (HEK) 293T cells were co-transfected with the assembled vector plasmid carrying the FAN1 gene, AAV helper plasmid, and an AAV PhP.eB rep/cap plasmid. Cells were harvested and lysed to release the AAV particles, which were then purified using iodixanol gradient ultracentrifugation. The purified viral vectors were titered via quantitative PCR.

### Retroorbital Injection

At approximately 4-5 weeks of age, R6/1 mice were anesthetized with isoflurane, and the purified AAV vectors were introduced via retroorbital injection, following the method outlined by Ramaswamy and Kordower (Challis, et al., Nature Protocols, Vol: 14 (2019), Issue: 2).

### Cell Transfection and Imaging:

SHSY5Y cells were grown in DMEM medium supplemented with 10% FBS and antibiotics. Cells were transfected with the pAAV plasmid constructs expressing miRFP670nano tagged FAN1 using the Mirus Trans-IT-2020 reagent, per the manufacturer's instructions. After a 48 hour period, cells were stained with Hoechst and CellMask for nuclei and plasma membranes, respectively, and imaged through confocal fluorescence microscopy.

### Western Blotting:

Cortical tissues were lysed using RIPA buffer with protease inhibitors. Protein concentrations were quantified using the Bradford assay. Equal amounts of protein were then separated by SDS-PAGE and transferred to nitrocellulose membranes. Membranes were incubated with a primary antibody against FAN1 and subsequently with a secondary HRP-conjugated antibody. Bands were visualized using enhanced chemiluminescence, and relative expression levels were determined through densitometric analysis.

### Electron Microscopy:

Tissue punchouts from mouse striatum were fixed and stained with heavy metals for contrast enhancement. Tissues were then dehydrated, infiltrated with resin, and polymerized. Ultrathin sections were prepared and imaged using a scanning electron microscope equipped with a focused ion beam. Analysis of these images provided insights into cellular differences and morphological changes.

### DNA Extraction and CAG Repeat Analysis:

DNA was extracted from striatal, liver, and cortical tissue of mice using the Zymo Quick-DNA column purification kit, following manufacturer's instructions. PCR amplification of the repeat region of the HTT gene was performed using FAM-labeled forward primer and a reverse primer. Fragment analysis was conducted through capillary electrophoresis using an ABI 3730 Genetic Analyzer, and the repeat sizes were determined via PeakScanner software.

### CAG Repeat Instability Index Quantification.

DNA from all mouse tissues was isolated using Zymo Quick-DNA column purification. CAG repeats were sized b PCR with primers

(SEQ ID NO: 22)
5′-(FAM label)-ATGAAGGCCTTCGAGTCCCTCAAGTCCTTC-3′

and

(SEQ ID NO: 23)
5′-GGCGGCTGAGGAAGCTGAGGA-3′

according to Mangiarini, et al., Cell, 1996 1; 87(3):493-506. with slight modifications. The FAM-labeled PCR products were mixed with GENESCAN™—1200 LIZ® Size Standard and HIDI™ Formamide (Applied Biosystems) and run on an ABI 3730 Genetic Analyzer (Applied Biosystems). Sizing of the PCR fragments was done by using the PeakScanner Software (Applied Biosystems).

Calculation of CAG repeat instability index was done with a custom script in Python as previously described Lee, et al., BMC Syst Biol. 2010; 4:29, with minor changes.

### Results

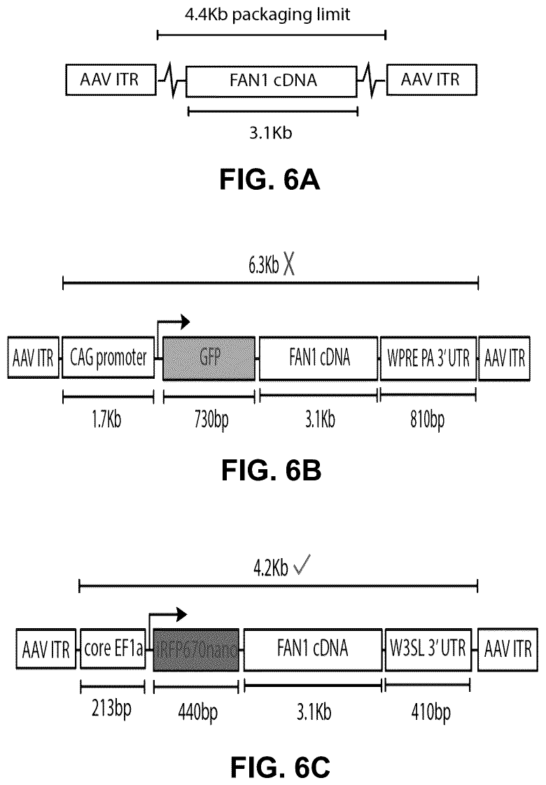

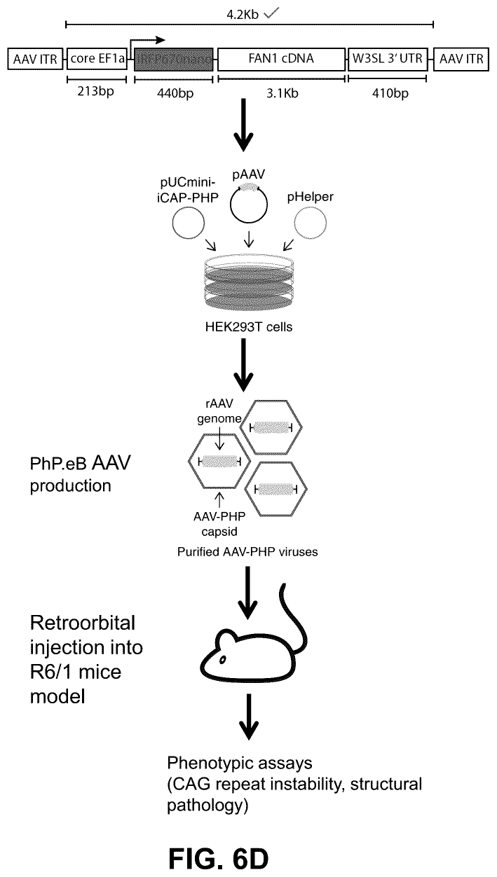

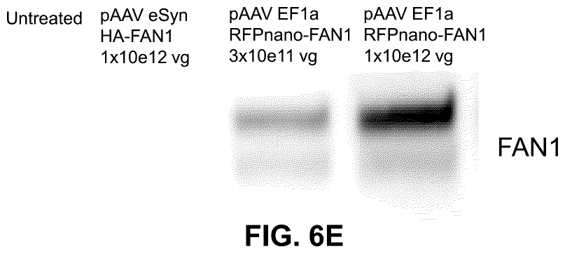

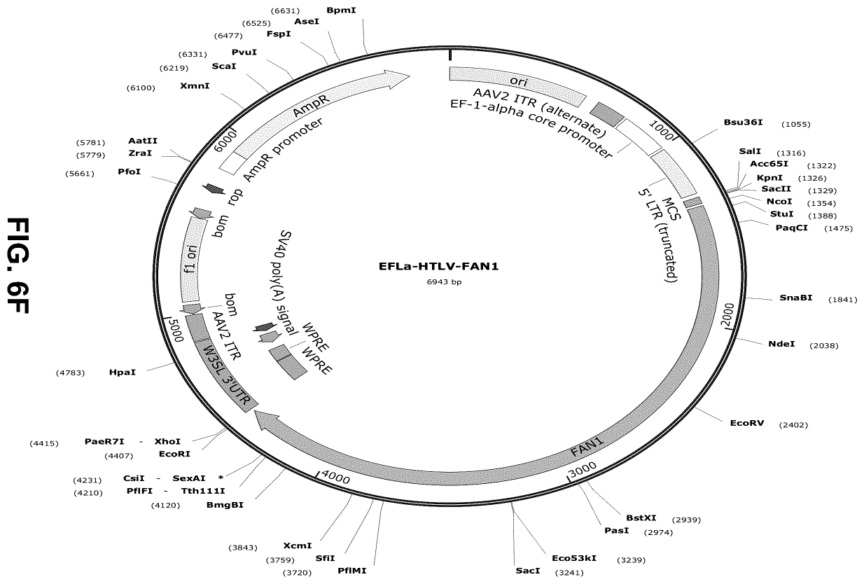

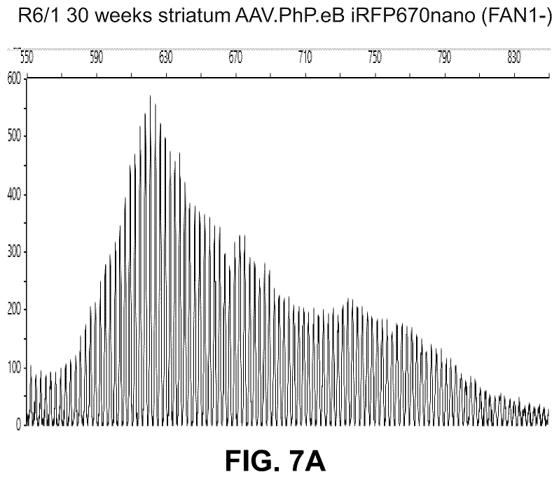

Overexpression of FAN1 has been difficult to accomplish thus far due to the behavior of this large FAN1 transgene in vivo. In the current study, overexpression of FAN1 by AAV.PhP.eB retroorbital injection in the R6/1 mice model of HD was achieved by efficient packaging of the large transgene (FIGS. 6A-6E). Cartoon illustration of optimized AAV expression cassette in comparison to traditional AAV cassette is shown in FIGS. 6A-6C. Overexpression of FAN1 by AAV is achieved by efficient packaging of the large 3.1 Kb transgene into an AAV cassette containing alternative ITR elements, a shortened 3′UTR poly-adenylation site and signal sequence (Choi, et al., Mol Brain. 2014; 7: 17), miniature transcriptional promoter (core EF1a/core EF1a+HTLV intron), and a small near-infrared (NIR) fluorescent protein tag (Oliinyk, et al., Nature Communications volume 10, Article number: 279 (2019)) at the N-terminus. Three types of FAN1 expressing AAV were constructed in these studies.
i) FAN1 overexpressing with core EF1a promoter and miRFP670nano fluorescent N-terminal tag (SEQ ID NO:1)
ii) FAN1 overexpressing with EF1a HTLV promoter and miRFP670nano fluorescent N-terminal tag (SEQ ID NO:2); and
iii) FAN1 overexpressing with EF1a HTLV promoter (no tag) (SEQ ID NO:3). HTLV is annotated as the 5′ LTR element downstream of the EF1a promoter

Localization of FAN1 to nuclear foci in SHSY5Y cells was assessed by fluorescence imaging (data not shown). The pAAV plasmid constructs expressing miRFP670nano tagged FAN1 were delivered to SHSY5Y cells using Mirus Trans-IT-2020 reagent according to manufacturer instructions. Confocal fluorescence imaging of the cells show the localization of miRFP670nano tagged FAN1 (red) primarily to nuclear foci. Hoescht (blue) and CellMask (green) staining mark nuclei and plasma membranes, respectively.

Western blot of cortical lysates confirmed FAN1 overexpression in mice using AAV.PhP.eB viral delivery (FIG. 6E). A combination of a miniature transcriptional promoter core EF1a, shortened 3′UTR W3 SL, and miRFP670nano N-terminal tag (SEQ ID: 1) is able to successfully overexpress FAN1 in mice. A human Synapsin promoter combined a short HA N-terminal tag does not overexpress FAN1 in mice.

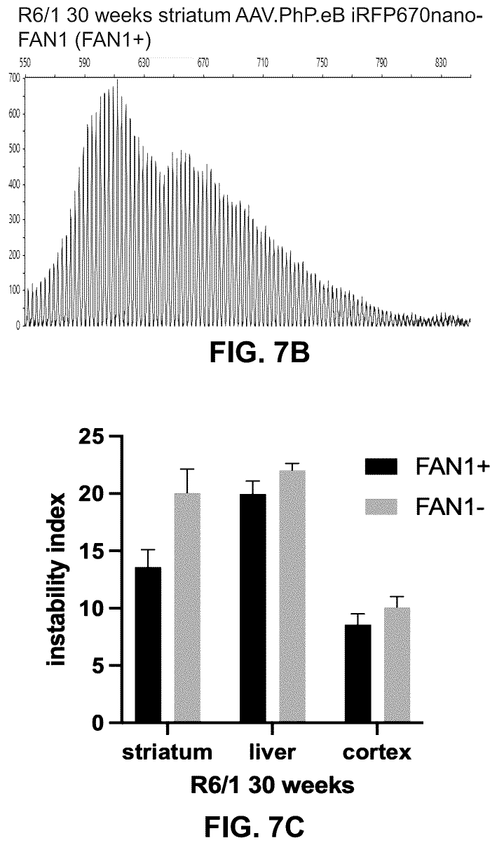

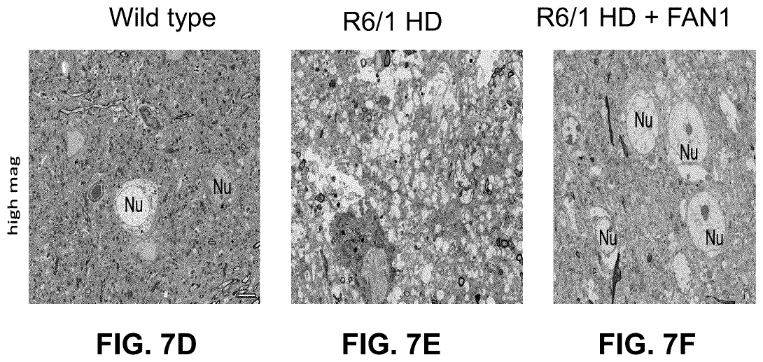

Overexpression of FAN1 by AAV.PhP.eB retroorbital injection rescued CAG repeat instability in R6/1 mice model of HD (FIG. 7A-7C). Rescue of CAG repeat instability was evaluated in striatal, liver, and cortical tissue by FAN1 overexpression in R6/1 mice by extraction of DNA followed by PCR of the repeat region of the HTT transgene and fragment analysis by capillary electrophoresis. The preliminary experiments indicate a reduced expansion of the CAG repeat in striatum from R6/1 mice at −30 weeks of age. Representative examples of PeakScanner raw data from the striatum of one FAN1-treated and one control R6/1 HD mouse at 30 weeks of age are shown (FIGS. 7A-7B).

The instability index calculated in striatum, cortex, and liver of FAN1-treated and control miRFP670-treated R6/1 mice (FIG. 7C). In order to measure the instability index, the magnitude of the peak heights included in the fragment analysis need to be normalized to one to the peak height of the main allele, multiplied by the distance to the main allele, and then summed.

Electron microscopy data showed potential rescue of structural pathology in R6/1 mice treated with FAN1 overexpression by PhP AAV (FIG. 7D-7F). At −15 weeks of age, tissue punchouts from mouse striatum were prepared by fixation and heavy metal staining and subsequently processed for resin embedding. A single slice of a 3D volume SEM image using a FIB-SEM shows dramatic cellular differences of three different mice: wild-type (WT), R6/1, and R6/1 overexpressing FAN1.

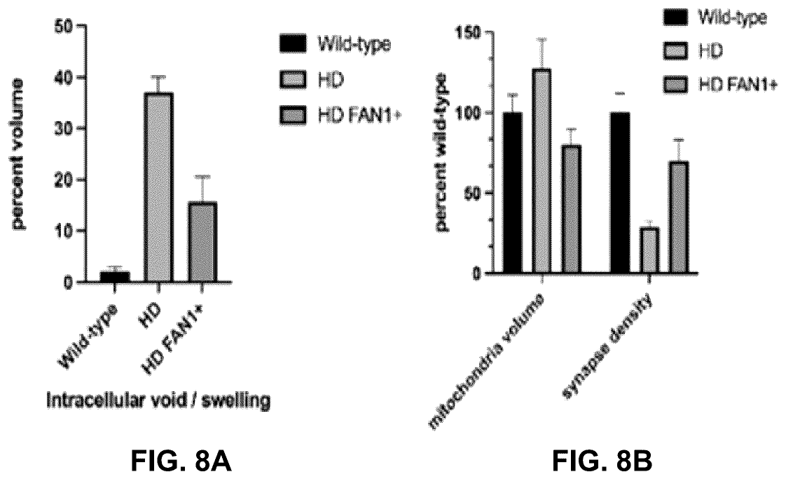

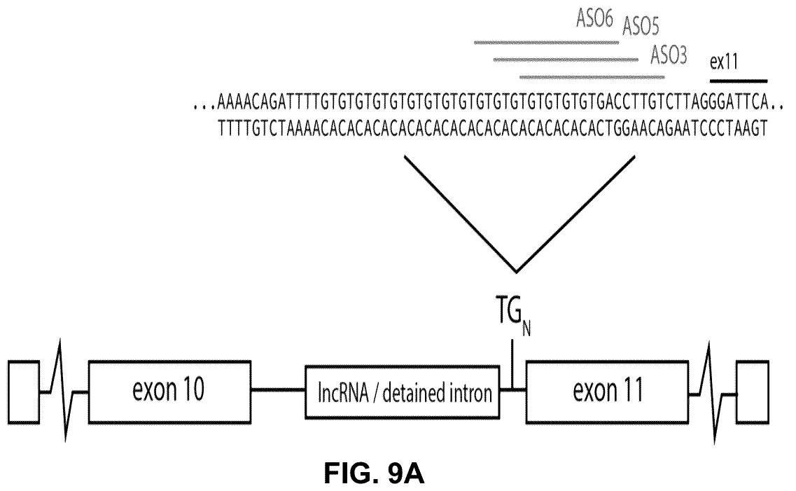

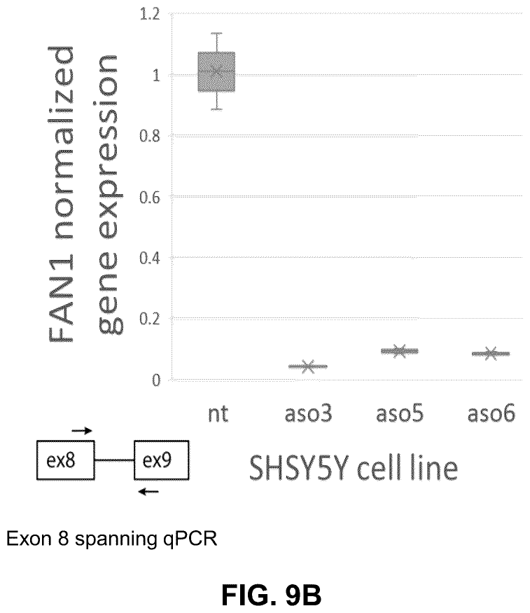

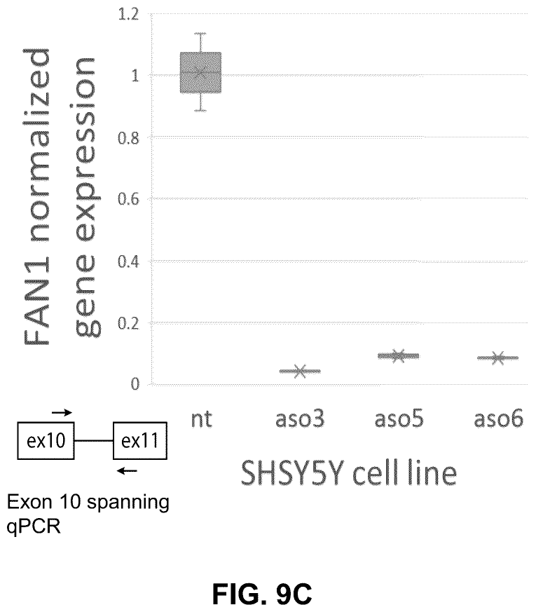

The EM approach included evaluation of the pathological changes by room temperature FIB-SEM slice and view of resin embedded striatal tissue from the R6/1 mice. Cellular components critical for neuronal function such as mitochondria, synapses, and autophagic vesicles were found to be structurally disrupted in the R6/1 striatum in comparison to a wild-type littermate. Overall, the R6/1 tissue is devoid of the intricate neuronal densities that are well organized in the multicellular neuronal network of the wild-type striatum. These structural defects in R6/1 mice are partially rescued by FAN1 overexpression by PhP.eB. Using Focused Ion Beam Scanning Electron Microscopy (FIB-SEM) of resin-embedded striatal tissue at room temperature, disruptions were observed in crucial cellular components, including mitochondria, synapses, and autophagic vesicles, in the R6/1 striatum compared to wild-type littermates. Specifically, the intricate, well-organized multicellular neuronal networks evident in the wild-type striatum were notably absent in the R6/1 tissue. Importantly, these structural defects were partially mitigated by FAN1 overexpression, suggesting a possible reversal of these pathological changes (FIGS. 8A-8B).

### Example 4: Design and Validation of a Codon Optimized FAN1 Protein Construct

### Materials and Methods

To enhance the expression of FAN1, two codon optimization strategies were adopted to increase FAN1 expression in-vivo (˜4 fold over the reference sequence of human FAN1).

Firstly, a consensus approach using AI-based tissue-specific algorithms and publicly available codon optimization tools was utilized to maximize protein production.

This consensus is an aligned DNA consensus sequence of seven publicly available codon optimization tools: GenSmart Optimization, Genewiz, IDT, ThermoFisher GeneArt GeneOptimizer, EO, Vectorbuilder and CUSTOM-AI.

The nucleic acid sequence for FAN1 was the consensus sequence:

(SEQ ID NO: 27)
ATGATGAGCGAGGGCAAGCCTCCAGACAAAAAGAGACCTAGAAGAAGCCTGTCCATCA
GCAAGAACAAGAAGAAGGCCAGCAACAGCATCATCTCTTGCTTTAACAACGCCCCACCTGCCA
AGCTGGCCTGTCCTGTTTGTAGCAAAATGGTGCCCAGATACGACCTGAATAGGCACCTGGACG
AAATGTGCGCCAACAACGACTTCGTGCAGGTCGACCCTGGCCAAGTGGGCCTGATCAACTCCA
ACGTGTCCATGGTGGATCTGACCTCTGTGACCCTGGAAGATGTGACCCCTAAGAAGTCCCCTC
CACCAAAGACAAACCTGACCCCTGGCCAGAGCGACTCCGCCAAGCGAGAAGTGAAGCAGAAGA
TCTCCCCCTACTTCAAGAGCAACGACGTGGTGTGCAAGAACCAGGACGAGCTGCGGAATCGGA
GCGTGAAAGTGATCTGCCTGGGCTCCCTGGCCTCCAAGCTGTCCCGAAAGTACGTGAAGGCCA
AGAAATCTATCGACAAGGACGAGGAATTCGCCGGCAGTTCCCCTCAGTCCTCCAAGTCCACCG
TGGTGAAGAGCCTGATCGACAATTCCTCCGAGATCGAGGACGAGGATCAGATCCTGGAGAACA
GCTCCCAGAAGGAGAACGTGTTTAAGTGTGACAGCCTGAAGGAGGAGTGCATCCCTGAGCACA
TGGTGAGAGGCAGCAAGATCATGGAAGCCGAGTCTCAGAAGGCCACCAGAGAATGCGAGAAGA
GTGCCCTGACACCAGGCTTCTCCGACAATGCCATCATGCTTTTCTCCCCCGATTTCACACTGA
GAAACACCCTGAAGTCCACCAGCGAGGACAGCCTGGTGAAGCAGGAGTGTATCAAAGAGGTGG
TGGAGAAGAGAGAGGCCTGTCACTGTGAGGAGGTGAAGATGACAGTGGCCTCCGAGGCCAAGA
TTCAGCTGTCAGACTCTGAAGCCAAGTCTCATAGCAGCGCCGATGATGCCTCTGCCTGGTCCA
ACATTCAGGAGGCCCCTCTGCAGGATGACAGCTGCCTGAACAACGATATCCCCCACAGCATCC
CTCTGGAACAGGGCAGCAGCTGTAATGGTCCTGGACAGACCACCGGGCATCCTTACTACCTGC
GCTCTTTCCTGGTGGTGCTGAAAACCGTGCTGGAGAACGAGGACGATATGCTGCTGTTCGACG
AGCAGGAGAAGGGCATCGTGACCAAGTTCTACCAGCTGTCCGCCACCGGCCAGAAGCTGTACG
TGCGGCTGTTTCAGAGAAAGCTGAGCTGGATCAAGATGACCAAACTGGAGTACGAAGAGATCG
CCCTGGACCTGACACCCGTGATCGAGGAGCTGACCAACGCCGGCTTTCTGCAGACCGAGTCCG
AGCTGCAGGAGCTGTCCGAAGTGCTGGAGCTGCTGAGCGCTCCTGAGCTGAAGAGCCTGGCCA
AGACCTTCCACCTGGTGAATCCCAACGGGCAGAAGCAGCAGCTGGTGGACGCCTTTCTGAAGC
TGGCCAAGCAGAGAAGCGTGTGCACCTGGGGCAAGAACAAGCCTGGCATCGGGGCCGTGATCT
TGAAAAGAGCCAAAGCCCTGGCCGGACAGTCCGTGCGCATCTGCAAGGGCCCTAGGGCCGTGT
TCAGCAGGATCTTGCTGTTGTTCTCCTTGACCGACTCCATGGAGGACGAGGACGCCGCATGTG
GCGGTCAGGGACAGCTGTCCACAGTGCTGCTGGTCAACCTGGGCAGAATGGAGTTCCCTAGTT
ACACCATCAACAGAAAAACCCACATCTTCCAGGACCGAGATGACCTGATCAGATACGCCGCCG
CCACCCATATGCTGAGCGACATCTCTTCCGCCATGGCCAATGGCAACTGGGAAGAGGCCAAGG
AACTGGCCCAGTGCGCCAAGAGAGATTGGAACAGGCTGAAGAACCACCCTTCCCTGAGATGCC
ACGAAGATCTGCCCCTGTTCCTGCGATGCTTCACCGTTGGCTGGATCTACACAAGAATCCTGA
GCCGGTTCGTGGAAATCCTGCAGAGGCTGCACATGTACGAGGAAGCCGTCAGAGAACTGGAGA
GCCTGCTGTCCCAGAGAATCTACTGCCCCGACTCCAGAGGCAGATGGTGGGACCGACTGGCCC
TTAATCTGCACCAGCACCTGAAGCGACTGGAGCCTACTATCAAGTGCATCACCGAGGGCCTGG
CCGACCCAGAGGTGCGAACCGGACACAGACTGAGCCTGTACCAGCGGGCCGTGCGACTGAGAG
AGAGCCCCAGCTGCAAGAAGTTCAAGCACCTGTTCCAGCAGCTGCCCGAGATGGCAGTGCAAG
ATGTGAAACACGTGACCATCACAGGCAGACTGTGCCCTCAGAGGGGCATGTGCAAGTCTGTGT
TTGTGATGGAAGCTGGCGAGGCCGCCGACCCCACCACAGTTCTGTGCAGCGTGGAGGAGCTGG
CTCTGGCCCACTACAGAAGATCCGGTTTCGACCAGGGCATTCACGGCGAGGGCAGCACATTTA
GCACCCTGTATGGCCTGCTGCTGTGGGACATCATCTTCATGGATGGGATCCCCGACGTGTTCA
GAAACGCCTGTCAGGCCTTCCCCCTGGACCTGTGCACCGACAGCTTCTTCACCTCCAGAAGAC
CCGCTCTGGAGGCCAGACTGCAACTGATCCACGACGCCCCTGAGGAGTCCCTGAGAGCCTGGG
TGGCCGCCACCTGGCACGAACAGGAGGGCAGAGTGGCTAGCCTGGTCTCCTGGGATAGATTCA
CCTCCCTGCAGCAGGCCCAGGATCTGGTGAGCTGCCTGGGAGGCCCCGTGCTGAGCGGCGTGT
GCAGACATCTGGCCGCCGATTTCCGACACTGCAGAGGCGGCCTTCCTGATCTGGTGGTGTGGA
ACAGCCAGAGCCGGCACTTTAAACTGGTCGAAGTGAAGGGCCCCAACGATCGACTGAGCCACA
AACAGATGATTTGGCTGGCCGAGCTGCAGAAACTGGGCGCCGAGGTGGAAGTGTGCCATGTGG
TGGCCGTGGGCGCCAAGTCTCAGTCCCTGAGCTAA;

The codon-optimized FAN1 nucleic acid sequence that was generated based on the AI-mediated customization protocol is:

(SEQ ID NO: 28)
ATGATGAGTGAGGGTAAGCCGCCAGATAAGAAGCGACCGAGACGTTCCCTAAGTATTT
CTAAAAATAAGAAAAAAGCCTCGAATTCGATCATCTCCTGCTTCAATAATGCACCACCAGCAA
AGCTCGCCTGTCCTGTTTGCTCCAAAATGGTGCCAAGATACGACTTAAATCGTCATCTAGACG
AAATGTGCGCAAACAATGATTTCGTCCAAGTCGATCCGGGCCAAGTAGGTCTAATTAATTCCA
ATGTCTCGATGGTTGACCTCACTTCTGTCACATTAGAAGACGTTACCCCCAAAAAGTCTCCTC
CGCCGAAGACTAATCTAACGCCAGGCCAGAGTGATAGTGCCAAACGAGAGGTAAAGCAGAAAA
TCTCTCCGTATTTTAAATCGAATGACGTTGTTTGTAAAAACCAGGACGAACTACGTAATCGTT
CGGTCAAGGTTATTTGCTTGGGATCCCTCGCTTCCAAGCTTTCGCGTAAATATGTCAAAGCAA
AGAAATCTATTGACAAGGATGAGGAGTTTGCCGGAAGTTCCCCTCAGTCCTCGAAGTCGACGG
TCGTAAAGAGTCTCATCGATAATTCGTCCGAAATCGAAGACGAAGATCAAATACTCGAAAACT
CTTCCCAAAAAGAAAACGTCTTCAAATGCGACAGTCTAAAAGAAGAGTGCATCCCCGAACACA
TGGTTAGAGGGTCGAAGATCATGGAAGCAGAGTCTCAGAAAGCAACGAGAGAATGCGAAAAGA
GTGCTTTGACACCGGGATTCTCGGACAATGCAATCATGTTATTTTCTCCAGATTTCACATTAC
GTAATACATTGAAGTCTACATCGGAGGATTCCCTCGTTAAGCAGGAATGCATTAAAGAAGTAG
TAGAGAAAAGAGAAGCATGTCACTGCGAAGAGGTTAAAATGACCGTGGCTTCTGAAGCTAAGA
TTCAGCTCTCCGACTCGGAAGCCAAATCTCATAGTAGTGCTGATGATGCCTCTGCCTGGTCGA
ATATTCAGGAAGCCCCACTCCAGGATGATTCGTGTCTGAACAATGATATACCGCATTCCATCC
CGCTTGAACAGGGCTCCTCCTGCAACGGCCCAGGACAGACTACTGGTCATCCTTACTATTTGC
GCAGTTTCCTTGTCGTCCTAAAAACTGTCCTCGAAAACGAAGACGATATGCTCTTATTTGACG
AACAAGAGAAAGGTATTGTCACGAAATTTTACCAACTCTCGGCTACTGGCCAAAAATTGTATG
TGAGATTATTTCAAAGAAAATTGAGTTGGATAAAAATGACAAAATTGGAATACGAAGAAATCG
CCCTCGACCTCACACCAGTCATTGAAGAACTCACAAATGCCGGATTTCTCCAGACTGAAAGTG
AATTGCAAGAGCTCTCTGAGGTACTCGAGCTACTCTCCGCTCCCGAGTTGAAATCCCTAGCCA
AAACATTCCATTTAGTAAATCCCAATGGACAAAAACAACAGTTGGTAGACGCCTTTTTAAAGC
TTGCCAAGCAAAGAAGTGTCTGCACGTGGGGAAAAAACAAGCCCGGCATCGGTGCCGTCATTT
TAAAACGTGCCAAGGCTTTAGCAGGCCAATCGGTCCGTATCTGCAAGGGTCCAAGAGCAGTGT
TCTCCCGTATCTTGCTACTCTTTTCGCTTACGGATTCCATGGAGGACGAAGATGCTGCATGCG
GAGGTCAGGGACAACTTTCGACGGTTTTATTGGTCAACCTCGGAAGAATGGAGTTCCCCTCTT
ACACCATAAATAGAAAAACCCACATTTTTCAAGATCGAGATGATCTCATCCGTTATGCTGCTG
CCACGCATATGCTAAGCGATATTAGTTCTGCTATGGCCAACGGAAATTGGGAAGAAGCCAAGG
AACTAGCACAATGCGCCAAGCGTGATTGGAACCGCCTAAAGAACCACCCTTCGTTGAGATGCC
ACGAAGACCTCCCACTCTTCTTAAGATGCTTCACTGTTGGCTGGATATATACAAGAATCCTAT
CTCGTTTCGTTGAAATCCTACAAAGACTACACATGTATGAAGAAGCCGTCCGTGAATTGGAGT
CGTTGCTTTCTCAGAGAATTTATTGTCCAGACTCTAGAGGAAGATGGTGGGACCGTCTCGCCC
TCAACCTTCATCAACACCTAAAGCGATTAGAGCCTACTATAAAATGCATTACAGAAGGACTTG
CAGACCCAGAAGTTCGTACAGGACACCGACTCTCCTTGTACCAAAGGGCAGTGCGATTGAGAG
AATCGCCCTCGTGCAAAAAATTTAAGCATCTATTCCAACAATTACCTGAAATGGCAGTTCAAG
ACGTGAAACATGTTACAATAACCGGCAGATTGTGCCCACAACGAGGAATGTGCAAATCGGTTT
TTGTAATGGAAGCCGGCGAGGCTGCAGACCCGACAACAGTCCTCTGTTCGGTCGAGGAACTAG
CCCTCGCACACTATCGTCGTTCCGGATTCGACCAAGGCATTCATGGAGAAGGTAGTACATTTT
CCACCCTATATGGATTACTCTTATGGGATATAATCTTCATGGACGGAATTCCAGATGTTTTCC
GAAACGCATGCCAGGCCTTTCCCCTAGACCTATGCACTGATAGTTTCTTCACCTCTAGAAGAC
CAGCTCTCGAGGCTCGCCTTCAATTGATTCATGATGCACCAGAAGAATCTCTCCGAGCTTGGG
TAGCCGCTACCTGGCATGAGCAGGAAGGAAGAGTTGCATCCTTAGTCTCGTGGGACCGTTTCA
CCTCTCTTCAACAAGCACAAGACTTGGTAAGTTGCCTAGGAGGTCCGGTCCTCTCTGGTGTCT
GCAGACATCTAGCCGCCGATTTCCGTCATTGCCGTGGTGGACTTCCTGATCTCGTAGTCTGGA
ATAGTCAATCGCGTCATTTCAAGTTGGTCGAGGTCAAGGGTCCCAACGATAGACTCAGTCATA
AACAAATGATTTGGCTCGCCGAACTACAAAAACTTGGTGCAGAAGTAGAAGTTTGCCATGTAG
TCGCTGTCGGAGCAAAATCCCAATCGTTATCCTAA.

### Results

Investigations into HD showcase the efficacy of FAN1 overexpression in neural tissue delivered by AAV PhP.eB for widespread CNS induction. The specifically designed an AAV expression cassette provided efficient FAN1 transgene packaging and overexpression. By optimizing the overexpression vector with several shortened elements and codon optimization, nuclear localization of FAN1 in cells was facilitated.

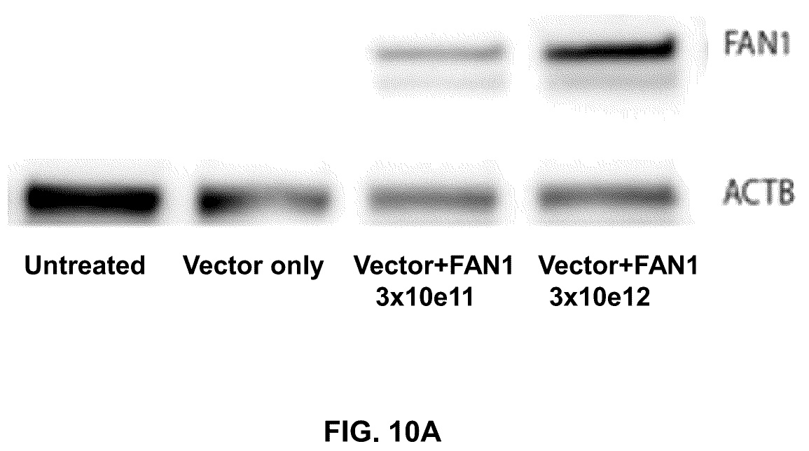

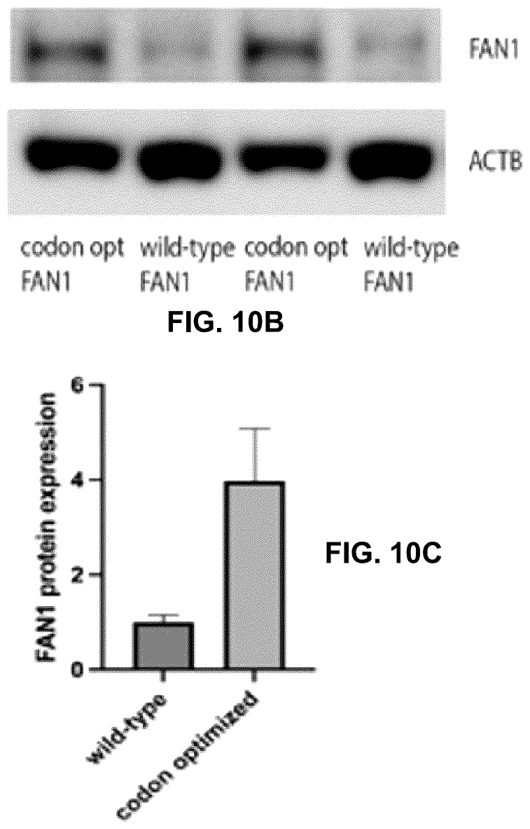

FAN1 overexpression in-vivo was confirmed via Western blot analysis of cortical lysates from mice, demonstrating the efficiency of the AAV retroorbital delivery system (FIGS. 10A-10C). The potential of FAN1 overexpression to modulate CAG repeat instability, a key pathological hallmark of HD, was further examined in the R6/1 mouse model. The findings showed a significant reduction in CAG repeat expansion in the striatum of FAN1-treated mice compared to the controls at around 30 weeks of age, detected through capillary electrophoresis of PCR-amplified HTT gene repeat regions from DNA samples.

### Example 5: Refinement of AAV System for Skeletal Muscle-Specific FAN1 Overexpression

### Aim

To adapt the AAV-mediated FAN1 overexpression strategy, previously successful in HD preclinical models, for targeting skeletal muscle pathology in DM1. By focusing on muscle tissue, to harness the advantages of AAV delivery in a more accessible and clinically translatable context, these studies aim to accelerate the development of effective therapies for DM1, HD and other REDs.

### Methods

Building upon the findings of these studies, to targeting skeletal muscle—a primary tissue affected by DM1, the MyoAAV capsid was employed for systemic muscle-directed gene delivery.

A comprehensive evaluation of the efficacy of various promoter constructs and localization tags was initiated. The “Myospreader” technology, which is designed to enhance gene delivery efficiency and distribution across the unique architecture of multinucleated muscle fibers, was integrated into the FAN1 expression vector system.

Cas9-delivering myoAAVs with either 2×SV40 NLS or Myospreader modifications are systemically administered to mdx mice to evaluate muscular tropism and expression efficiency.

The Myospreader technology leverages a fusion protein approach to promote the widespread intracellular distribution of the therapeutic protein, ensuring comprehensive coverage within the muscle tissue. Constructs were designed to include:

(i) the MHCK7 promoter, (ii) NES-NoLS Myospreader, (iii) FAN1 cDNA, and (iv) W3SL 3′UTR, all flanked by AAV ITRs (see, construct design depicted in FIGS. 11A-11B, showing schematics of two AAV constructs: FIG. 11A is a control construct with core EF1a promoter; FIG. 11B construct includes MHCK7-Myospreader construct optimized for muscle-specific expression. Western blot analysis was used to assess FAN1 protein expression levels in quadriceps tissue post-MyoAAV injection.

### Results

Comparative Analysis of AAV Vector Designs for Muscular Gene Delivery indicated that AAV with only nuclear localization sequence (NLS) leads to limited mRNA and protein export, while AAV with both NLS and nuclear export sequences (Myospreader) ensures broader intracellular mRNA and protein distribution.

By Western blot quantification in quadriceps tissue of mice after retro-orbital administration of MyoAAVs, the muscle-specific MHCK7 promoter, combined with the Myospreader localization tag, markedly outperformed our original FAN1 AAV vector (see FIG. 11C, demonstrating the vastly improved expression with MHCK7-Myospreader construct over the control EF1a vectors.)

This successful overexpression of FAN1 in-vivo exemplifies the potential for specifically tailored vectors to treat muscular pathologies in REDs. The promising results with the Myospreader technology as part of our AAV delivery system indicate a significant advancement in our ability to address the distribution challenges in muscular gene therapy, setting a new precedent for the treatment of myotonic dystrophy.

### Example 6: Targeting TDP-43 to Increase FAN1 Splicing/Expression Levels

It was observed that knockdown of TDP-43, a splicing regulator known to bind to UG repeats, also results in increased FAN1 expression.

### Methods

### TDP-43 Inhibition

Reducing the expression or activity of TDP-43 alleviates its regulatory effects on splicing, thereby promoting increased FAN1 splicing and expression. This was observed in-vitro using an ASO gapmer having the nucleic acid sequence:

AAGGCttcatattgtACTTT (SEQ ID NO: 26; Capital
letter is 2′MOE modified; small letter is DNA
unmodified)

to knockdown TDP-43 in SH-SY5Y neuroblastoma cells.

### Results

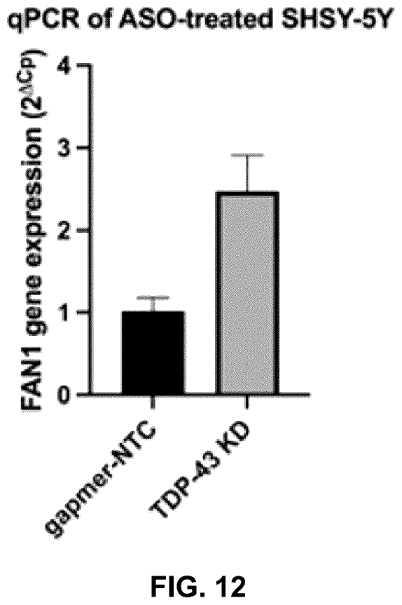

TDP-43 knockdown using an ASO gapmer in SH-SY5Y cells elevates FAN1 gene expression, as shown in FIG. 12. The discovery that knockdown of TDP-43 enhances FAN1 splicing and expression levels, indicates a regulatory mechanism that can be exploited for therapeutic purposes.

By exploring these innovative approaches, the data advances the understanding of splicing regulation within the FAN1 gene and identifies new targets for therapeutic intervention in repeat expansion disorders.

1. A composition comprising: (a) a splice switching antisense oligonucleotide (SSASO) or (b) an antisense oligonucleotide (ASO) that targets a pre-mRNA encoding FANCD2 And FANCI Associated Nuclease 1 (FAN1) comprising an intron 10 and an exon 11
wherein the ASO or SSASO comprises from about 8 to about 50 nucleotides, inclusive, complementary to a sequence within intron 10 of a pre-mRNA encoding FANCD2 And FANCI Associated Nuclease 1 (FAN1), optionally, comprising one or more chemically modified nucleotides; optionally in a pharmaceutically acceptable carrier for administration in vivo.

2. The composition of claim 1, comprising from about 8 to about 50 nucleotides, inclusive, complementary to a sequence within the nucleic acid sequence of SEQ ID NO:2.

3. The composition of claim 1, comprising from about 8 to about 22 nucleotides, inclusive, complementary to the nucleic acid sequence of any one of SEQ ID NOs:4-6.

4. The composition of claim 1, comprising the nucleotide sequence of any one of SEQ ID NOs:7-9 or 32.

5. The composition of claim 1 comprising an ASO that targets a pre-mRNA encoding FANCD2 And FANCI Associated Nuclease 1 (FAN1) comprising an intron 10 and an exon 11.

6. The composition of claim 5, wherein the ASO comprises from about 8 to about 20 nucleotides, inclusive, complementary to a nucleic acid sequence as set forth in SEQ ID NO:25.

7. The composition of claim 5, comprising the nucleotide sequence of any one of SEQ ID NOs:10-12.

8. The composition of claim 1, comprising a chemical modification, wherein the chemical modification comprises phosphorothiate (PS) backbone modification, and/or modification at the 2′ sugar position.

9. The composition of claim 8, wherein the modification at the 2′ sugar position is selected from the group consisting of 2′-O-methyl (2′-OMe) and 2′-O-methoxyethyl (2′-MOE).

10. A nucleic acid composition comprising a codon optimized nucleic acid sequence encoding a FANCD2 And FANCI Associated Nuclease 1 (FAN1) protein, optionally, in a pharmaceutically acceptable carrier for administration in vivo.

11. The composition of claim 9, wherein the codon optimized nucleic acid sequence comprises the nucleic acid sequence set forth in SEQ ID NO:28 and/or wherein the nucleic acid comprises an expression vector.

12. The composition of claim 11, wherein the expression vector comprises an Adeno-associated Virus (AAV).

13. The composition of claim 9, wherein the AAV is configured for expression within the muscles and/or neurons in vivo.

14. The composition of claim 9, comprising the nucleic acid sequence set forth in SEQ ID NO:21.

15. A method of ameliorating or delaying the onset of a trinucleotide repeat expansion disease or disorder in a subject in need thereof comprising increasing the amount of mRNA and/or protein expression of FANCD2 And FANCI Associated Nuclease 1 (FAN1) in the subject,
wherein the subject has a FAN1 pre-mRNA comprising an intron 10, and
wherein the intron 10 comprises more than 17 contiguous dinucleotide repeats of thymine and guanine (TG), optionally, wherein the trinucleotide repeat expansion disorder is Huntington's disease, cerebrospinal ataxia, or fragile X syndrome.

16. The method of claim 15, wherein the intron 10 comprises 18, 19, 20, 21, 22, 23, or more than 23 contiguous dinucleotide TG repeats.

17. The method of claim 15, wherein:
(a) the subject has a reduced amount of FAN1 mRNA and/or FAN1 protein, as compared to a healthy control subject;
(b) wherein the step of increasing the amount of FAN1 mRNA and/or FAN1 protein comprises administering to the subject a pharmaceutical composition, comprising:
(i) a splice switching antisense oligonucleotide (SSASO) comprising from about 8 to about 50 nucleotides, inclusive, wherein the ASO comprises from about 8 to about 50 nucleotides, inclusive, complementary to a sequence within intron 10 of a pre-mRNA encoding FANCD2 And FANCI Associated Nuclease 1 (FAN1), optionally, comprising one or more chemically modified nucleotides; or
(ii) an antisense oligonucleotide (ASO) that targets a pre-mRNA encoding FANCD2 And FANCI Associated Nuclease 1 (FAN1) comprising an intron 10 and an exon 11,
wherein the ASO comprises from about 8 to about 20 nucleotides, inclusive, complementary to a splice junction between the intron 10, and the exon 11, optionally, comprising one or more chemically modified nucleotides
in an amount effective to increase levels of functional FAN1 mRNA transcripts encoding FAN1 derived from the pre-mRNA.

18. The method of claim 15, wherein:
(a) the composition is administered in an amount effective to increase the level of mRNA and/or protein expression of FAN1 in one or more neuronal cells compared to the level prior to the administration of the composition; (b) the step of increasing the level of mRNA and/or protein expression of FAN1 is achieved by CRISPR/Cas based gene editing, optionally, wherein the CRISPR/Cas based gene editing comprises Cas13 and an sgRNA; optionally wherein the method further comprises one or more steps for reducing or preventing the expression and/or amount of TDP-43 in the subject.

19. A minigene reporter system comprising
(a) the nucleotide sequence of exon 10, intron 10 and exon 11 of a FAN1pre-mRNA, optionally wherein
(i) the nucleotide sequence of exon 10 comprises SEQ ID NO:1; and/or
(ii) the nucleotide sequence of intron 10 comprises SEQ ID NO:2; and/or
(iii) the nucleotide sequence of exon 11 comprises SEQ ID NO:3; and
(b) a reporter gene,
wherein the minigene is configured such that the reporter gene is expressed only when the nucleotide sequence of intron 10 is spliced from the minigene, optionally, wherein the reporter gene encodes enhanced green fluorescent protein.

20. A method for identifying one or more splice modulators that increase levels of functional FAN1 mRNA transcripts encoding FAN1 derived from the pre-mRNA in one or more neuronal cells comprising
(i) contacting the minigene reporter system of claim 19 and one or more candidate molecules, and
(ii) determining the level of reporter gene expression, and
(iii) selecting the candidate molecules that increase the level of reporter gene expression as suitable for increasing the level of mRNA and/or protein expression of FAN1 in one or more neuronal cells.
